(19) United States
(12) Patent Application Publication
(10) Pub. No.: US 2012/0227119 A1
(43) Pub. Date: Sep. 6, 2012

(54) Methods of Modulating the Sex of Avians

(75) Inventors: Timothy James Doran, Ocean Grove (AU); Robert John Moore, Ascot Vale (AU); John William Lowenthal, Belmont (AU); Craig Smith, Parkville (AU); Andrew Henrik Sinclair, South Melbourne (AU)

(73) Assignees: Australian Poultry CRC Pty Ltd, Armidale, New South Wales (AU); Commonwealth Scientific and Industrial Research Organisation, Campbell, ACT (AU)

(21) Appl. No.: 13/139,163

(22) PCT Filed: Dec. 16, 2009
(86) PCT No.: PCT/AU2009/001627

§ 371 Date: Dec. 21, 2011

Related U.S. Application Data
(60) Provisional application No. 61/138,235, filed on Dec. 17, 2008.

(51) Int. Cl. A01K 67/027; A61K 31/713

(52) U.S. Cl. 800/8; 514/44 A

(57) ABSTRACT

The present invention relates to nucleic acids and methods for modulating the sex of avians. In particular, the invention relates to the in ovo delivery of a dsRNA molecule, especially siRNAs, to increase the production of female birds.

### FIELD OF THE INVENTION

The present invention relates to nucleic acids and methods for modulating the sex of avians. In particular, the invention relates to the in ovo delivery of a dsRNA molecule, especially siRNAs, to increase the production of female birds.

### BACKGROUND OF THE INVENTION

Man has modified the phenotypic characteristics of domestic animals through selection of seed stock over many generations ever since animals were domesticated. This has led to improvements in quantitative production parameters such as body size and muscle mass. More recent innovations of modifying production traits of poultry and/or improving resistance to pathogens has focussed on transgenic approaches, however, many consumers have concerns about genetically modified organisms.

Chicken producers have been searching for an efficient, economical method of determining the sex of day old chicks. Vent sexing and feather sexing have been used by the various producers, but these methods have been found to have substantial economic disadvantages because of the substantial time required and labour costs in separating the male from the female chicks. The use of probes (U.S. Pat. No. 5,508,165) is also an expensive procedure and not practical economically. Light sensing of anal areas of chicks (U.S. Pat. No. 4,417,663) is another way of determining sex of chicks, but it is also expensive and time consuming as each chick must be handled and manipulated. The use of experts who could feather sex the chicks has been used, but such experts are costly and feathering is time consuming.

There is a need for nucleic acids and methods for modifying avian sex that do not result in transformation of the bird's genome, but are amenable to high throughout processing.

### SUMMARY OF THE INVENTION

The present inventors have identified nucleic acid molecules, in particular dsRNA molecules, which can be used to modify the sex of avians in ovo.

Accordingly, in one aspect the present invention provides an isolated and/or exogenous nucleic acid molecule comprising a double-stranded region which reduces the level of at least one RNA molecule and/or protein when administered to an avian egg, wherein if the embryo of the egg is male the sex is altered to female following administration of the isolated and/or exogenous nucleic acid molecule, and wherein the isolated and/or exogenous nucleic acid molecule does not comprise a sequence selected from:

### CCAGUUGUCAAGAAGAGCA (SEQ ID NO:254)

### GGAUGCUCAUUCAGGACAU (SEQ ID NO:369)

### CCCUGUAUCCUUACUAUAA (SEQ ID NO:474)

### GCCACUGAGUCUUCCUCAA (SEQ ID NO:530)

### CCAGCAACAUACAUGUCAA (SEQ ID NO:605)

### CCUGCGUCACACAGAUACU (SEQ ID NO:747)

### GGAGUAGUUGUACAGGUUG (SEQ ID NO:3432)

### GACUGGCUUGACAUGUAUG (SEQ ID NO:3433)

AUGGCGGUUCUCCAUCCCU (SEQ ID NO:3434) or a variant of any one thereof.

In another aspect, the present invention provides an isolated and/or exogenous nucleic acid molecule comprising one or more of the sequence of nucleotides provided as SEQ ID NO's 11 to 3431 or a variant of any one or more thereof, wherein the isolated and/or exogenous nucleic acid molecule does not comprise a sequence selected from:

### CCAGUUGUCAAGAAGAGCA (SEQ ID NO:254)

### GGAUGCUCAUUCAGGACAU (SEQ ID NO:369)

### CCCUGUAUCCUUACUAUAA (SEQ ID NO:474)

### GCCACUGAGUCUUCCUCAA (SEQ ID NO:530)

### CCAGCAACAUACAUGUCAA (SEQ ID NO:605)

### CCUGCGUCACACAGAUACU (SEQ ID NO:747)

### GGAGUAGUUGUACAGGUUG (SEQ ID NO:3432)

### GACUGGCUUGACAUGUAUG (SEQ ID NO:3433)

AUGGCGGUUCUCCAUCCCU (SEQ ID NO:3434) or a variant of any one thereof.

In a preferred embodiment, the nucleic acid molecule is dsRNA. More preferably, the dsRNA is a siRNA or a shRNA.

In a preferred embodiment, the nucleic acid molecule reduces the level of a protein encoded by a DMRT1 gene, ASW gene or r-spondin gene, in an avian egg.

In a preferred embodiment of the two above aspects, the nucleic acid does not comprise the full length open reading frame of the RNA molecule or the cDNA encoding therefor. In a related embodiment, preferably the nucleic acid does not comprise a sequence of nucleotides provided as SEQ ID NO's 2, 4 or 6, or a sequence of nucleotides provided as SEQ ID NO's 2, 4 or 6 where each T (thymine) is replaced with a U (uracil).

As the skilled addressee will appreciate, because the nucleic acid is double stranded it will also comprise the corresponding reverse complement of the relevant nucleotide sequence provided herewith.

Also provided is a vector encoding a nucleic acid molecule, or a single strand thereof, according to the invention. Such vectors can be used in a host cell or cell-free expression system to produce nucleic acid molecules useful for the method of the invention.

In another aspect, the present invention provides a host cell comprising an exogenous nucleic acid molecule, or a single strand thereof, of the invention and/or a vector of the invention.

In another aspect, the present invention provides a composition comprising a nucleic acid molecule, or a single strand thereof, of the invention, a vector of the invention, and/or a host cell of the invention.

In a further aspect, the present invention provides a method of modifying the sex of an avian, the method comprising administering to an avian egg at least one nucleic acid molecule of the invention.

Preferably, the nucleic acid is administered to a non-cellular site of the egg. More preferably, the non-cellular site is the air sac, yolk sac, amnionic cavity or chorion allantoic fluid.

In a further preferred embodiment, the egg is not electroporated.

Preferably, the nucleic acid is not delivered by administering a vector encoding the nucleic acid molecule.

Preferably, the nucleic acid molecule administered is dsRNA.

Preferably, the nucleic acid molecule is administered by injection.

Conveniently, the nucleic acid may be administered in a composition of the invention.

In a preferred embodiment, the method modifies the sex of the embryo of the egg from male to female.

The avian can be any species of the Class Ayes. Examples include, but are not limited to, chickens, ducks, turkeys, geese, bantams and quails. In a particularly preferred embodiment, the avian is a chicken.

In a further aspect, the present invention provides an avian produced using a method of the invention.

In another aspect, the present invention provides a chicken produced using a method of the invention.

In a further aspect, the present invention provides an avian egg comprising a nucleic acid molecule, or a single strand thereof, of the invention, a vector of the invention, and/or a host cell of the invention.

In another aspect, the present invention provides a kit comprising a nucleic acid molecule, or a single strand thereof, of the invention, a vector of the invention, a host cell of the invention, and/or a composition of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Selected shRNAs for knockdown of EGFP-Dmrt1 gene fusion expression. Mean fluorescence intensity for each transfection condition expressed relative to pEGFP-Dmrt1. Error bars indicate standard error calculated on each individual experiment completed in triplicate.

FIG. 2—qPCR analysis of DMRT1 gene expression following silencing.

### KEY TO THE SEQUENCE LISTING

SEQ ID NO:1—Partial chicken DMRT1 protein sequence (Genbank AF123456).
SEQ ID NO:2—Partial nucleotide sequence encoding chicken DMRT1 (Genbank AF123456).

### SEQ ID NO:3—Chicken WPKCI (ASW) (Genbank AF148455).

SEQ ID NO:4—Nucleotide sequence encoding chicken WPKCI (ASW) (Genbank AF148455).
SEQ ID NO:5—Chicken r-spondin (Genbank XM_{—}417760).
SEQ ID NO:6—Nucleotide sequence encoding chicken r-spondin (Genbank XM_{—}417760).
SEQ ID NO:7—Nucleotide sequence of chicken U6-1 promoter.
SEQ ID NO:8—Nucleotide sequence of chicken U6-3 promoter.
SEQ ID NO:9—Nucleotide sequence of chicken U6-4 promoter.
SEQ ID NO:10—Nucleotide sequence of chicken 7SK promoter.
SEQ ID NO's 11 to 3430—RNA sequences provided in Tables 1 to 3 for silencing the chicken DMRT1, ASW or r-spondin genes.
SEQ ID NO's 3431 to 3434—RNA sequences suitable for silencing the chicken DMRT1 gene.
SEQ ID NO's 3435 to 3485—Target regions of chicken DMRT1.
SEQ ID NO's 3486 to 3499—Oligonucleotide primers and probes.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, avian biology, RNA interference, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors).

The term “avian” as used herein refers to any species, subspecies or race of organism of the taxonomic Class Ayes, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, California Gray, Italian Partidge-coloured, as well as strains of turkeys, pheasants, quails, duck, ostriches and other poultry commonly bred in commercial quantities.

As used herein, the term “egg” refers to a fertilized ovum that has been laid by a bird. Typically, avian eggs consist of a hard, oval outer eggshell, the “egg white” or albumen, the egg yolk, and various thin membranes. Furthermore, “in ovo” refers to in an egg.

As used herein, the term “non-cellular site” refers a part of the egg other than the embryo.

The terms “reduces”, “reduction” or variations thereof as used herein refers to a measurable decrease in the amount of a target RNA and/or target protein in the egg when compared to an egg from the same species of avian, more preferably strain or breed of avian, and even more preferably the same bird, that has not been administered with a nucleic acid as defined herein. The term also refers to a measurable reduction in the activity of a target protein. Preferably a reduction in the level of a target RNA and/or target protein is at least about 10%. More preferably, the reduction is at least about 20%, 30%, 40%, 50%, 60%, 80%, 90% and even more preferably, about 100%.

As used herein, the phrase “the nucleic acid molecule results in a reduction” or variations thereof refers to the presence of the nucleic acid molecule in the egg inducing degradation of homologous RNAs in the egg by the process known in the art as “RNA interference” or “gene silencing”. Furthermore, the nucleic acid molecule directly results in the reduction, and is not transcribed in ovo to produce the desired effect.

The “at least one RNA molecule” can be any type of RNA present in, and/or produced by, an avian egg. Examples include, but are not limited to, mRNA, snRNA, microRNA and tRNA.

A “variant” of a nucleic acid molecule of the invention includes molecules of varying sizes of, and/or with one or more different nucleotides, but which are still capable of being used to silence the target gene. For example, variants may comprise additional nucleotides (such as 1, 2, 3, 4, or more), or less nucleotides. Furthermore, a few nucleotides may be substituted without influencing the ability of the nucleic acid to silence the target gene. In an embodiment, the variant includes additional 5′ and/or 3′ nucleotides which are homologous to the corresponding target RNA molecule and/or which enhance the stability of the nucleic acid molecule. In another embodiment, the nucleic acid molecules have no more than 4, more preferably no more than 3, more preferably no more than 2, and even more preferably no more than 1, nucleotide differences when compared to the sequences provided herein. In a further embodiment, the nucleic acid molecules have no more than 2, and more preferably no more than 1, internal additional and/or deletional nucleotides when compared to the sequences provided herein. In an embodiment, a nucleic acid of the invention has one, preferably two, additional non-target nucleotides at the 5′ and/or 3′ end, for example an additional UU at the 3′ end. Such additions can increase the half-life of the molecule in ovo.

By an “isolated nucleic acid molecule”, we mean a nucleic acid molecule which is at least partially separated from the nucleic acid molecule with which it is associated or linked in its native state. Preferably, the isolated nucleic acid molecule is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a nucleic acid molecule refers to the nucleic acid molecule when present in a cell, or in a cell-free expression system, in an altered amount. Preferably, the cell is a cell that does not naturally comprise the nucleic acid molecule. However, the cell may be a cell which comprises an exogenous nucleic acid molecule resulting in an increased amount of the nucleic acid molecule. An exogenous nucleic acid molecule of the invention includes nucleic acid molecules which have not been separated from other components of the recombinant cell, or cell-free expression system, in which it is present, and nucleic acid molecules produced in such cells or cell-free systems which are subsequently purified away from at least some other components.

### Sex Determination

The present invention relates to the modulation of the sex of avians in ovo. Examples of genes which can be targeted to modulate avian sex include, but are not necessarily limited to, the DMRT1 gene, the ASW (WPKCI) gene, the R-spondin gene, the Fox9 gene and the β-catenin gene.

In a preferred embodiment, the nucleic acid molecule reduces the level of a protein encoded by a DMRT1 gene. DMRT1 was the first molecule implicated in sex determination that shows sequence conservation between phyla. The avian homologue of DMRT1 is found on the Z (sex) chromosome of chickens and is differentially expressed in the genital ridges of male and female chicken embryos (Raymond et al., 1999; Smith et al., 1999). DMRT1 is one of the few genes thus far implicated in mammalian sex determination that appears to have a strictly gonadal pattern of expression (Raymond et al., 1999).

Examples of nucleic acid molecules that can be used to reduce the level of chicken DMRT1 protein, and mRNA encoding therefor, include, but are not limited to, nucleic acids comprising one or more of the sequence of nucleotides provided in Table 1 (SEQ ID NO's 11 to 1644), or a variant of any one or more thereof, with the exception that the nucleic acid molecule does not comprise a sequence selected from:

### CCAGUUGUCAAGAAGAGCA (SEQ ID NO:254)

### GGAUGCUCAUUCAGGACAU (SEQ ID NO:369)

### CCCUGUAUCCUUACUAUAA (SEQ ID NO:474)

### GCCACUGAGUCUUCCUCAA (SEQ ID NO:530)

### CCAGCAACAUACAUGUCAA (SEQ ID NO:605)

### CCUGCGUCACACAGAUACU (SEQ ID NO:747)

GGAGUAGUUGUACAGGUUG (SEQ ID NO:3432) (reverse complement of SEQ ID NO:493)
GACUGGCUUGACAUGUAUG (SEQ ID NO:3433) (reverse complement of SEQ ID NO:612)
AUGGCGGUUCUCCAUCCCU (SEQ ID NO:3434) (reverse complement of SEQ ID NO:1520), or a variant of any one thereof.

In a particularly preferred embodiment, the nucleic acid molecule that can be used to reduce the level of chicken DMRT1 protein comprises a sequence selected from; GAGCCAGUUGUCAAGAAGA (SEQ ID NO:251), GACUGCCAGUGCAAGAAGU (SEQ ID NO:116), CUGUAUCCUUACUAUAACA (SEQ ID NO:476), and CUCCCAGCAACAUACAUGU (SEQ ID NO:602), or a variant of any one thereof. More preferably, the nucleic acid molecule that can be used to reduce the level of chicken DMRT1 protein comprises the sequence, GAGCCAGUUGUCAAGAAGA (SEQ ID NO:251), or a variant thereof such as GAGCCAGUUGUCAAGAAGAUU (SEQ ID NO:3431).

A further example of a gene that can be targeted to modify sex is the WPKCI gene. The avian gene WPKCI has been shown to be conserved widely on the avian W chromosome and expressed actively in the female chicken embryo before the onset of gonadal differentiation. It is suggested that WPKCI may play a role in the differentiation of the female gonad by interfering with the function of PKCI or by exhibiting its unique function in the nucleus (Hori et al., 2000). This gene has also been identified as ASW (avian sex-specific W-linked) (O'Neill et al., 2000).

Examples of nucleic acid molecules that can be used to reduce the level of chicken ASW (WPKCI) protein, and mRNA encoding therefor, include, but are not limited to, nucleic acids comprising one or more of the sequence of nucleotides provided in Table 2 (SEQ ID NO's 1645 to 2209), or a variant of any one or more thereof.

In yet another example of a gene that can be targeted to modify sex is the r-spondin gene. Examples of nucleic acid molecules that can be used to reduce the level of chicken r-spondin protein, and mRNA encoding therefor, include, but are not limited to, nucleic acids comprising one or more of the sequence of nucleotides provided in Table 3 (SEQ ID NO's 2210 to 3430), or a variant of any one or more thereof.

### Gene Silencing

The terms “RNA interference”, “RNAi” or “gene silencing” refers generally to a process in which a double-stranded RNA (dsRNA) molecule reduces the expression of a nucleic acid sequence with which the double-stranded RNA molecule shares substantial or total homology. However, it has more recently been shown that gene silencing can be achieved using non-RNA double stranded molecules (see, for example, US 20070004667).

RNA interference (RNAi) is particularly useful for specifically inhibiting the production of a particular RNA and/or protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide of interest. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029 and WO 01/34815.

The present invention includes nucleic acid molecules comprising and/or encoding double-stranded regions for gene silencing. The nucleic acid molecules are typically RNA but may comprise DNA, chemically-modified nucleotides and non-nucleotides.

TABLE 1
DsRNA molecules targeting mRNA encoding chicken
DMRT1.

| SEQ ID NO | Sequence 5′-3′ |
|---|---|
| 11 | CCGGCGGCGGGCAAGAAGC |
| 12 | CGGCGGCGGGCAAGAAGCU |
| 13 | GGCGGCGGGCAAGAAGCUG |
| 14 | GCGGCGGGCAAGAAGCUGC |
| 15 | CGGCGGGCAAGAAGCUGCC |
| 16 | GGCGGGCAAGAAGCUGCCG |
| 17 | GCGGGCAAGAAGCUGCCGC |
| 18 | CGGGCAAGAAGCUGCCGCG |
| 19 | GGGCAAGAAGCUGCCGCGU |
| 20 | GGCAAGAAGCUGCCGCGUC |
| 21 | GCAAGAAGCUGCCGCGUCU |
| 22 | CAAGAAGCUGCCGCGUCUG |
| 23 | AAGAAGCUGCCGCGUCUGC |
| 24 | AGAAGCUGCCGCGUCUGCC |
| 25 | GAAGCUGCCGCGUCUGCCC |
| 26 | AAGCUGCCGCGUCUGCCCA |
| 27 | AGCUGCCGCGUCUGCCCAA |
| 28 | GCUGCCGCGUCUGCCCAAG |
| 29 | CUGCCGCGUCUGCCCAAGU |
| 30 | UGCCGCGUCUGCCCAAGUG |
| 31 | GCCGCGUCUGCCCAAGUGU |
| 32 | CCGCGUCUGCCCAAGUGUG |
| 33 | CGCGUCUGCCCAAGUGUGC |
| 34 | GCGUCUGCCCAAGUGUGCC |
| 35 | CGUCUGCCCAAGUGUGCCC |
| 36 | GUCUGCCCAAGUGUGCCCG |
| 37 | UCUGCCCAAGUGUGCCCGC |
| 38 | CUGCCCAAGUGUGCCCGCU |
| 39 | UGCCCAAGUGUGCCCGCUG |
| 40 | GCCCAAGUGUGCCCGCUGC |
| 41 | CCCAAGUGUGCCCGCUGCC |
| 42 | CCAAGUGUGCCCGCUGCCG |
| 43 | CAAGUGUGCCCGCUGCCGC |
| 44 | AAGUGUGCCCGCUGCCGCA |
| 45 | AGUGUGCCCGCUGCCGCAA |
| 46 | GUGUGCCCGCUGCCGCAAC |
| 47 | UGUGCCCGCUGCCGCAACC |
| 48 | GUGCCCGCUGCCGCAACCA |
| 49 | UGCCCGCUGCCGCAACCAC |
| 50 | GCCCGCUGCCGCAACCACG |
| 51 | CCCGCUGCCGCAACCACGG |
| 52 | CCGCUGCCGCAACCACGGC |
| 53 | CGCUGCCGCAACCACGGCU |
| 54 | GCUGCCGCAACCACGGCUA |
| 55 | CUGCCGCAACCACGGCUAC |
| 56 | UGCCGCAACCACGGCUACU |
| 57 | GCCGCAACCACGGCUACUC |
| 58 | CCGCAACCACGGCUACUCC |
| 59 | CGCAACCACGGCUACUCCU |
| 60 | GCAACCACGGCUACUCCUC |
| 61 | CAACCACGGCUACUCCUCG |
| 62 | AACCACGGCUACUCCUCGC |
| 63 | ACCACGGCUACUCCUCGCC |
| 64 | CCACGGCUACUCCUCGCCG |
| 65 | CACGGCUACUCCUCGCCGC |
| 66 | ACGGCUACUCCUCGCCGCU |
| 67 | CGGCUACUCCUCGCCGCUG |
| 68 | GGCUACUCCUCGCCGCUGA |
| 69 | GCUACUCCUCGCCGCUGAA |
| 70 | CUACUCCUCGCCGCUGAAG |
| 71 | UACUCCUCGCCGCUGAAGG |
| 72 | ACUCCUCGCCGCUGAAGGG |
| 73 | CUCCUCGCCGCUGAAGGGG |
| 74 | UCCUCGCCGCUGAAGGGGC |
| 75 | CCUCGCCGCUGAAGGGGCA |
| 76 | CUCGCCGCUGAAGGGGCAC |
| 77 | UCGCCGCUGAAGGGGCACA |
| 78 | CGCCGCUGAAGGGGCACAA |
| 79 | GCCGCUGAAGGGGCACAAG |
| 80 | CCGCUGAAGGGGCACAAGC |
| 81 | CGCUGAAGGGGCACAAGCG |
| 82 | GCUGAAGGGGCACAAGCGG |
| 83 | CUGAAGGGGCACAAGCGGU |
| 84 | UGAAGGGGCACAAGCGGUU |
| 85 | GAAGGGGCACAAGCGGUUC |
| 86 | AAGGGGCACAAGCGGUUCU |
| 87 | AGGGGCACAAGCGGUUCUG |
| 88 | GGGGCACAAGCGGUUCUGC |
| 89 | GGGCACAAGCGGUUCUGCA |
| 90 | GGCACAAGCGGUUCUGCAU |
| 91 | GCACAAGCGGUUCUGCAUG |
| 92 | CACAAGCGGUUCUGCAUGU |
| 93 | ACAAGCGGUUCUGCAUGUG |
| 94 | CAAGCGGUUCUGCAUGUGG |
| 95 | AAGCGGUUCUGCAUGUGGC |
| 96 | AGCGGUUCUGCAUGUGGCG |
| 97 | GCGGUUCUGCAUGUGGCGG |
| 98 | CGGUUCUGCAUGUGGCGGG |
| 99 | GGUUCUGCAUGUGGCGGGA |
| 100 | GUUCUGCAUGUGGCGGGAC |
| 101 | UUCUGCAUGUGGCGGGACU |
| 102 | UCUGCAUGUGGCGGGACUG |
| 103 | CUGCAUGUGGCGGGACUGC |
| 104 | UGCAUGUGGCGGGACUGCC |
| 105 | GCAUGUGGCGGGACUGCCA |
| 106 | CAUGUGGCGGGACUGCCAG |
| 107 | AUGUGGCGGGACUGCCAGU |
| 108 | UGUGGCGGGACUGCCAGUG |
| 109 | GUGGCGGGACUGCCAGUGC |
| 110 | UGGCGGGACUGCCAGUGCA |
| 111 | GGCGGGACUGCCAGUGCAA |
| 112 | GCGGGACUGCCAGUGCAAG |
| 113 | CGGGACUGCCAGUGCAAGA |
| 114 | GGGACUGCCAGUGCAAGAA |
| 115 | GGACUGCCAGUGCAAGAAG |
| 116 | GACUGCCAGUGCAAGAAGU |
| 117 | ACUGCCAGUGCAAGAAGUG |
| 118 | CUGCCAGUGCAAGAAGUGC |
| 119 | UGCCAGUGCAAGAAGUGCA |
| 120 | GCCAGUGCAAGAAGUGCAG |
| 121 | CCAGUGCAAGAAGUGCAGC |
| 122 | CAGUGCAAGAAGUGCAGCC |
| 123 | AGUGCAAGAAGUGCAGCCU |
| 124 | GUGCAAGAAGUGCAGCCUG |
| 125 | UGCAAGAAGUGCAGCCUGA |
| 126 | GCAAGAAGUGCAGCCUGAU |
| 127 | CAAGAAGUGCAGCCUGAUC |
| 128 | AAGAAGUGCAGCCUGAUCG |
| 129 | AGAAGUGCAGCCUGAUCGC |
| 130 | GAAGUGCAGCCUGAUCGCC |
| 131 | AAGUGCAGCCUGAUCGCCG |
| 132 | AGUGCAGCCUGAUCGCCGA |
| 133 | GUGCAGCCUGAUCGCCGAG |
| 134 | UGCAGCCUGAUCGCCGAGC |
| 135 | GCAGCCUGAUCGCCGAGCG |
| 136 | CAGCCUGAUCGCCGAGCGG |
| 137 | AGCCUGAUCGCCGAGCGGC |
| 138 | GCCUGAUCGCCGAGCGGCA |
| 139 | CCUGAUCGCCGAGCGGCAG |
| 140 | CUGAUCGCCGAGCGGCAGC |
| 141 | UGAUCGCCGAGCGGCAGCG |
| 142 | GAUCGCCGAGCGGCAGCGG |
| 143 | AUCGCCGAGCGGCAGCGGG |
| 144 | UCGCCGAGCGGCAGCGGGU |
| 145 | CGCCGAGCGGCAGCGGGUG |
| 146 | GCCGAGCGGCAGCGGGUGA |
| 147 | CCGAGCGGCAGCGGGUGAU |
| 148 | CGAGCGGCAGCGGGUGAUG |
| 149 | GAGCGGCAGCGGGUGAUGG |
| 150 | AGCGGCAGCGGGUGAUGGC |
| 151 | GCGGCAGCGGGUGAUGGCC |
| 152 | CGGCAGCGGGUGAUGGCCG |
| 153 | GGCAGCGGGUGAUGGCCGU |
| 154 | GCAGCGGGUGAUGGCCGUG |
| 155 | CAGCGGGUGAUGGCCGUGC |
| 156 | AGCGGGUGAUGGCCGUGCA |
| 157 | GCGGGUGAUGGCCGUGCAG |
| 158 | CGGGUGAUGGCCGUGCAGG |
| 159 | GGGUGAUGGCCGUGCAGGU |
| 160 | GGUGAUGGCCGUGCAGGUU |
| 161 | GUGAUGGCCGUGCAGGUUG |
| 162 | UGAUGGCCGUGCAGGUUGC |
| 163 | GAUGGCCGUGCAGGUUGCA |
| 164 | AUGGCCGUGCAGGUUGCAC |
| 165 | UGGCCGUGCAGGUUGCACU |
| 166 | GGCCGUGCAGGUUGCACUG |
| 167 | GCCGUGCAGGUUGCACUGA |
| 168 | CCGUGCAGGUUGCACUGAG |
| 169 | CGUGCAGGUUGCACUGAGG |
| 170 | GUGCAGGUUGCACUGAGGA |
| 171 | UGCAGGUUGCACUGAGGAG |
| 172 | GCAGGUUGCACUGAGGAGG |
| 173 | CAGGUUGCACUGAGGAGGC |
| 174 | AGGUUGCACUGAGGAGGCA |
| 175 | GGUUGCACUGAGGAGGCAG |
| 176 | GUUGCACUGAGGAGGCAGC |
| 177 | UUGCACUGAGGAGGCAGCA |
| 178 | UGCACUGAGGAGGCAGCAA |
| 179 | GCACUGAGGAGGCAGCAAG |
| 180 | CACUGAGGAGGCAGCAAGC |
| 181 | ACUGAGGAGGCAGCAAGCC |
| 182 | CUGAGGAGGCAGCAAGCCC |
| 183 | UGAGGAGGCAGCAAGCCCA |
| 184 | GAGGAGGCAGCAAGCCCAG |
| 185 | AGGAGGCAGCAAGCCCAGG |
| 186 | GGAGGCAGCAAGCCCAGGA |
| 187 | GAGGCAGCAAGCCCAGGAA |
| 188 | AGGCAGCAAGCCCAGGAAG |
| 189 | GGCAGCAAGCCCAGGAAGA |
| 190 | GCAGCAAGCCCAGGAAGAG |
| 191 | CAGCAAGCCCAGGAAGAGG |
| 192 | AGCAAGCCCAGGAAGAGGA |
| 193 | GCAAGCCCAGGAAGAGGAG |
| 194 | CAAGCCCAGGAAGAGGAGC |
| 195 | AAGCCCAGGAAGAGGAGCU |
| 196 | AGCCCAGGAAGAGGAGCUG |
| 197 | GCCCAGGAAGAGGAGCUGG |
| 198 | CCCAGGAAGAGGAGCUGGG |
| 199 | CCAGGAAGAGGAGCUGGGG |
| 200 | CAGGAAGAGGAGCUGGGGA |
| 201 | AGGAAGAGGAGCUGGGGAU |
| 202 | GGAAGAGGAGCUGGGGAUC |
| 203 | GAAGAGGAGCUGGGGAUCA |
| 204 | AAGAGGAGCUGGGGAUCAG |
| 205 | AGAGGAGCUGGGGAUCAGC |
| 206 | GAGGAGCUGGGGAUCAGCC |
| 207 | AGGAGCUGGGGAUCAGCCA |
| 208 | GGAGCUGGGGAUCAGCCAC |
| 209 | GAGCUGGGGAUCAGCCACC |
| 210 | AGCUGGGGAUCAGCCACCC |
| 211 | GCUGGGGAUCAGCCACCCU |
| 212 | CUGGGGAUCAGCCACCCUG |
| 213 | UGGGGAUCAGCCACCCUGU |
| 214 | GGGGAUCAGCCACCCUGUA |
| 215 | GGGAUCAGCCACCCUGUAC |
| 216 | GGAUCAGCCACCCUGUACC |
| 217 | GAUCAGCCACCCUGUACCC |
| 218 | AUCAGCCACCCUGUACCCC |
| 219 | UCAGCCACCCUGUACCCCU |
| 220 | CAGCCACCCUGUACCCCUG |
| 221 | AGCCACCCUGUACCCCUGC |
| 222 | GCCACCCUGUACCCCUGCC |
| 223 | CCACCCUGUACCCCUGCCC |
| 224 | CACCCUGUACCCCUGCCCA |
| 225 | ACCCUGUACCCCUGCCCAG |
| 226 | CCCUGUACCCCUGCCCAGU |
| 227 | CCUGUACCCCUGCCCAGUG |
| 228 | CUGUACCCCUGCCCAGUGC |
| 229 | UGUACCCCUGCCCAGUGCC |
| 230 | GUACCCCUGCCCAGUGCCC |
| 231 | UACCCCUGCCCAGUGCCCC |
| 232 | ACCCCUGCCCAGUGCCCCU |
| 233 | CCCCUGCCCAGUGCCCCUG |
| 234 | CCCUGCCCAGUGCCCCUGA |
| 235 | CCUGCCCAGUGCCCCUGAG |
| 236 | CUGCCCAGUGCCCCUGAGC |
| 237 | UGCCCAGUGCCCCUGAGCC |
| 238 | GCCCAGUGCCCCUGAGCCA |
| 239 | CCCAGUGCCCCUGAGCCAG |
| 240 | CCAGUGCCCCUGAGCCAGU |
| 241 | CAGUGCCCCUGAGCCAGUU |
| 242 | AGUGCCCCUGAGCCAGUUG |
| 243 | GUGCCCCUGAGCCAGUUGU |
| 244 | UGCCCCUGAGCCAGUUGUC |
| 245 | GCCCCUGAGCCAGUUGUCA |
| 246 | CCCCUGAGCCAGUUGUCAA |
| 247 | CCCUGAGCCAGUUGUCAAG |
| 248 | CCUGAGCCAGUUGUCAAGA |
| 249 | CUGAGCCAGUUGUCAAGAA |
| 250 | UGAGCCAGUUGUCAAGAAG |
| 251 | GAGCCAGUUGUCAAGAAGA |
| 252 | AGCCAGUUGUCAAGAAGAG |
| 253 | GCCAGUUGUCAAGAAGAGC |
| 254 | CCAGUUGUCAAGAAGAGCA |
| 255 | CAGUUGUCAAGAAGAGCAG |
| 256 | AGUUGUCAAGAAGAGCAGC |
| 257 | GUUGUCAAGAAGAGCAGCA |
| 258 | UUGUCAAGAAGAGCAGCAG |
| 259 | UGUCAAGAAGAGCAGCAGC |
| 260 | GUCAAGAAGAGCAGCAGCA |
| 261 | UCAAGAAGAGCAGCAGCAG |
| 262 | CAAGAAGAGCAGCAGCAGC |
| 263 | AAGAAGAGCAGCAGCAGCA |
| 264 | AGAAGAGCAGCAGCAGCAG |
| 265 | GAAGAGCAGCAGCAGCAGC |
| 266 | AAGAGCAGCAGCAGCAGCU |
| 267 | AGAGCAGCAGCAGCAGCUC |
| 268 | GAGCAGCAGCAGCAGCUCC |
| 269 | AGCAGCAGCAGCAGCUCCU |
| 270 | GCAGCAGCAGCAGCUCCUG |
| 271 | CAGCAGCAGCAGCUCCUGU |
| 272 | AGCAGCAGCAGCUCCUGUC |
| 273 | GCAGCAGCAGCUCCUGUCU |
| 274 | CAGCAGCAGCUCCUGUCUC |
| 275 | AGCAGCAGCUCCUGUCUCC |
| 276 | GCAGCAGCUCCUGUCUCCU |
| 277 | CAGCAGCUCCUGUCUCCUG |
| 278 | AGCAGCUCCUGUCUCCUGC |
| 279 | GCAGCUCCUGUCUCCUGCA |
| 280 | CAGCUCCUGUCUCCUGCAG |
| 281 | AGCUCCUGUCUCCUGCAGG |
| 282 | GCUCCUGUCUCCUGCAGGA |
| 283 | CUCCUGUCUCCUGCAGGAC |
| 284 | UCCUGUCUCCUGCAGGACA |
| 285 | CCUGUCUCCUGCAGGACAG |
| 286 | CUGUCUCCUGCAGGACAGC |
| 287 | UGUCUCCUGCAGGACAGCA |
| 288 | GUCUCCUGCAGGACAGCAG |
| 289 | UCUCCUGCAGGACAGCAGC |
| 290 | CUCCUGCAGGACAGCAGCA |
| 291 | UCCUGCAGGACAGCAGCAG |
| 292 | CCUGCAGGACAGCAGCAGC |
| 293 | CUGCAGGACAGCAGCAGCC |
| 294 | UGCAGGACAGCAGCAGCCC |
| 295 | GCAGGACAGCAGCAGCCCU |
| 296 | CAGGACAGCAGCAGCCCUG |
| 297 | AGGACAGCAGCAGCCCUGC |
| 298 | GGACAGCAGCAGCCCUGCU |
| 299 | GACAGCAGCAGCCCUGCUC |
| 300 | ACAGCAGCAGCCCUGCUCA |
| 301 | CAGCAGCAGCCCUGCUCAC |
| 302 | AGCAGCAGCCCUGCUCACU |
| 303 | GCAGCAGCCCUGCUCACUC |
| 304 | CAGCAGCCCUGCUCACUCC |
| 305 | AGCAGCCCUGCUCACUCCA |
| 306 | GCAGCCCUGCUCACUCCAC |
| 307 | CAGCCCUGCUCACUCCACG |
| 308 | AGCCCUGCUCACUCCACGA |
| 309 | GCCCUGCUCACUCCACGAG |
| 310 | CCCUGCUCACUCCACGAGC |
| 311 | CCUGCUCACUCCACGAGCA |
| 312 | CUGCUCACUCCACGAGCAC |
| 313 | UGCUCACUCCACGAGCACG |
| 314 | GCUCACUCCACGAGCACGG |
| 315 | CUCACUCCACGAGCACGGU |
| 316 | UCACUCCACGAGCACGGUG |
| 317 | CACUCCACGAGCACGGUGG |
| 318 | ACUCCACGAGCACGGUGGC |
| 319 | CUCCACGAGCACGGUGGCA |
| 320 | UCCACGAGCACGGUGGCAG |
| 321 | CCACGAGCACGGUGGCAGC |
| 322 | CACGAGCACGGUGGCAGCA |
| 323 | ACGAGCACGGUGGCAGCAG |
| 324 | CGAGCACGGUGGCAGCAGC |
| 325 | GAGCACGGUGGCAGCAGCA |
| 326 | AGCACGGUGGCAGCAGCAG |
| 327 | GCACGGUGGCAGCAGCAGC |
| 328 | CACGGUGGCAGCAGCAGCA |
| 329 | ACGGUGGCAGCAGCAGCAG |
| 330 | CGGUGGCAGCAGCAGCAGC |
| 331 | GGUGGCAGCAGCAGCAGCG |
| 332 | GUGGCAGCAGCAGCAGCGA |
| 333 | UGGCAGCAGCAGCAGCGAG |
| 334 | GGCAGCAGCAGCAGCGAGC |
| 335 | GCAGCAGCAGCAGCGAGCG |
| 336 | CAGCAGCAGCAGCGAGCGC |
| 337 | AGCAGCAGCAGCGAGCGCA |
| 338 | GCAGCAGCAGCGAGCGCAC |
| 339 | CAGCAGCAGCGAGCGCACC |
| 340 | AGCAGCAGCGAGCGCACCA |
| 341 | GCAGCAGCGAGCGCACCAC |
| 342 | CAGCAGCGAGCGCACCACC |
| 343 | AGCAGCGAGCGCACCACCA |
| 344 | GCAGCGAGCGCACCACCAG |
| 345 | CAGCGAGCGCACCACCAGA |
| 346 | AGCGAGCGCACCACCAGAG |
| 347 | GCGAGCGCACCACCAGAGG |
| 348 | CGAGCGCACCACCAGAGGG |
| 349 | GAGCGCACCACCAGAGGGA |
| 350 | AGCGCACCACCAGAGGGAC |
| 351 | GCGCACCACCAGAGGGACG |
| 352 | CGCACCACCAGAGGGACGG |
| 353 | GCACCACCAGAGGGACGGA |
| 354 | CACCACCAGAGGGACGGAU |
| 355 | ACCACCAGAGGGACGGAUG |
| 356 | CCACCAGAGGGACGGAUGC |
| 357 | CACCAGAGGGACGGAUGCU |
| 358 | ACCAGAGGGACGGAUGCUC |
| 359 | CCAGAGGGACGGAUGCUCA |
| 360 | CAGAGGGACGGAUGCUCAU |
| 361 | AGAGGGACGGAUGCUCAUU |
| 362 | GAGGGACGGAUGCUCAUUC |
| 363 | AGGGACGGAUGCUCAUUCA |
| 364 | GGGACGGAUGCUCAUUCAG |
| 365 | GGACGGAUGCUCAUUCAGG |
| 366 | GACGGAUGCUCAUUCAGGA |
| 367 | ACGGAUGCUCAUUCAGGAC |
| 368 | CGGAUGCUCAUUCAGGACA |
| 369 | GGAUGCUCAUUCAGGACAU |
| 370 | GAUGCUCAUUCAGGACAUC |
| 371 | AUGCUCAUUCAGGACAUCC |
| 372 | UGCUCAUUCAGGACAUCCC |
| 373 | GCUCAUUCAGGACAUCCCU |
| 374 | CUCAUUCAGGACAUCCCUU |
| 375 | UCAUUCAGGACAUCCCUUC |
| 376 | CAUUCAGGACAUCCCUUCC |
| 377 | AUUCAGGACAUCCCUUCCA |
| 378 | UUCAGGACAUCCCUUCCAU |
| 379 | UCAGGACAUCCCUUCCAUC |
| 380 | CAGGACAUCCCUUCCAUCC |
| 381 | AGGACAUCCCUUCCAUCCC |
| 382 | GGACAUCCCUUCCAUCCCC |
| 383 | GACAUCCCUUCCAUCCCCA |
| 384 | ACAUCCCUUCCAUCCCCAG |
| 385 | CAUCCCUUCCAUCCCCAGC |
| 386 | AUCCCUUCCAUCCCCAGCA |
| 387 | UCCCUUCCAUCCCCAGCAG |
| 388 | CCCUUCCAUCCCCAGCAGA |
| 389 | CCUUCCAUCCCCAGCAGAG |
| 390 | CUUCCAUCCCCAGCAGAGG |
| 391 | UUCCAUCCCCAGCAGAGGG |
| 392 | UCCAUCCCCAGCAGAGGGC |
| 393 | CCAUCCCCAGCAGAGGGCA |
| 394 | CAUCCCCAGCAGAGGGCAC |
| 395 | AUCCCCAGCAGAGGGCACU |
| 396 | UCCCCAGCAGAGGGCACUU |
| 397 | CCCCAGCAGAGGGCACUUG |
| 398 | CCCAGCAGAGGGCACUUGG |
| 399 | CCAGCAGAGGGCACUUGGA |
| 400 | CAGCAGAGGGCACUUGGAG |
| 401 | AGCAGAGGGCACUUGGAGA |
| 402 | GCAGAGGGCACUUGGAGAG |
| 403 | CAGAGGGCACUUGGAGAGC |
| 404 | AGAGGGCACUUGGAGAGCA |
| 405 | GAGGGCACUUGGAGAGCAC |
| 406 | AGGGCACUUGGAGAGCACG |
| 407 | GGGCACUUGGAGAGCACGU |
| 408 | GGCACUUGGAGAGCACGUC |
| 409 | GCACUUGGAGAGCACGUCU |
| 410 | CACUUGGAGAGCACGUCUG |
| 411 | ACUUGGAGAGCACGUCUGA |
| 412 | CUUGGAGAGCACGUCUGAU |
| 413 | UUGGAGAGCACGUCUGAUU |
| 414 | UGGAGAGCACGUCUGAUUU |
| 415 | GGAGAGCACGUCUGAUUUG |
| 416 | GAGAGCACGUCUGAUUUGG |
| 417 | AGAGCACGUCUGAUUUGGU |
| 418 | GAGCACGUCUGAUUUGGUU |
| 419 | AGCACGUCUGAUUUGGUUG |
| 420 | GCACGUCUGAUUUGGUUGU |
| 421 | CACGUCUGAUUUGGUUGUG |
| 422 | ACGUCUGAUUUGGUUGUGG |
| 423 | CGUCUGAUUUGGUUGUGGA |
| 424 | GUCUGAUUUGGUUGUGGAC |
| 425 | UCUGAUUUGGUUGUGGACU |
| 426 | CUGAUUUGGUUGUGGACUC |
| 427 | UGAUUUGGUUGUGGACUCC |
| 428 | GAUUUGGUUGUGGACUCCA |
| 429 | AUUUGGUUGUGGACUCCAC |
| 430 | UUUGGUUGUGGACUCCACC |
| 431 | UUGGUUGUGGACUCCACCU |
| 432 | UGGUUGUGGACUCCACCUA |
| 433 | GGUUGUGGACUCCACCUAC |
| 434 | GUUGUGGACUCCACCUACU |
| 435 | UUGUGGACUCCACCUACUA |
| 436 | UGUGGACUCCACCUACUAC |
| 437 | GUGGACUCCACCUACUACA |
| 438 | UGGACUCCACCUACUACAG |
| 439 | GGACUCCACCUACUACAGC |
| 440 | GACUCCACCUACUACAGCA |
| 441 | ACUCCACCUACUACAGCAG |
| 442 | CUCCACCUACUACAGCAGU |
| 443 | UCCACCUACUACAGCAGUU |
| 444 | CCACCUACUACAGCAGUUU |
| 445 | CACCUACUACAGCAGUUUU |
| 446 | ACCUACUACAGCAGUUUUU |
| 447 | CCUACUACAGCAGUUUUUA |
| 448 | CUACUACAGCAGUUUUUAC |
| 449 | UACUACAGCAGUUUUUACC |
| 450 | ACUACAGCAGUUUUUACCA |
| 451 | CUACAGCAGUUUUUACCAG |
| 452 | UACAGCAGUUUUUACCAGC |
| 453 | ACAGCAGUUUUUACCAGCC |
| 454 | CAGCAGUUUUUACCAGCCA |
| 455 | AGCAGUUUUUACCAGCCAU |
| 456 | GCAGUUUUUACCAGCCAUC |
| 457 | CAGUUUUUACCAGCCAUCC |
| 458 | AGUUUUUACCAGCCAUCCC |
| 459 | GUUUUUACCAGCCAUCCCU |
| 460 | UUUUUACCAGCCAUCCCUG |
| 461 | UUUUACCAGCCAUCCCUGU |
| 462 | UUUACCAGCCAUCCCUGUA |
| 463 | UUACCAGCCAUCCCUGUAU |
| 464 | UACCAGCCAUCCCUGUAUC |
| 465 | ACCAGCCAUCCCUGUAUCC |
| 466 | CCAGCCAUCCCUGUAUCCU |
| 467 | CAGCCAUCCCUGUAUCCUU |
| 468 | AGCCAUCCCUGUAUCCUUA |
| 469 | GCCAUCCCUGUAUCCUUAC |
| 470 | CCAUCCCUGUAUCCUUACU |
| 471 | CAUCCCUGUAUCCUUACUA |
| 472 | AUCCCUGUAUCCUUACUAU |
| 473 | UCCCUGUAUCCUUACUAUA |
| 474 | CCCUGUAUCCUUACUAUAA |
| 475 | CCUGUAUCCUUACUAUAAC |
| 476 | CUGUAUCCUUACUAUAACA |
| 477 | UGUAUCCUUACUAUAACAA |
| 478 | GUAUCCUUACUAUAACAAC |
| 479 | UAUCCUUACUAUAACAACC |
| 480 | AUCCUUACUAUAACAACCU |
| 481 | UCCUUACUAUAACAACCUG |
| 482 | CCUUACUAUAACAACCUGU |
| 483 | CUUACUAUAACAACCUGUA |
| 484 | UUACUAUAACAACCUGUAC |
| 485 | UACUAUAACAACCUGUACA |
| 486 | ACUAUAACAACCUGUACAA |
| 487 | CUAUAACAACCUGUACAAC |
| 488 | UAUAACAACCUGUACAACU |
| 489 | AUAACAACCUGUACAACUA |
| 490 | UAACAACCUGUACAACUAC |
| 491 | AACAACCUGUACAACUACU |
| 492 | ACAACCUGUACAACUACUC |
| 493 | CAACCUGUACAACUACUCC |
| 494 | AACCUGUACAACUACUCCC |
| 495 | ACCUGUACAACUACUCCCA |
| 496 | CCUGUACAACUACUCCCAG |
| 497 | CUGUACAACUACUCCCAGU |
| 498 | UGUACAACUACUCCCAGUA |
| 499 | GUACAACUACUCCCAGUAC |
| 500 | UACAACUACUCCCAGUACC |
| 501 | ACAACUACUCCCAGUACCA |
| 502 | CAACUACUCCCAGUACCAA |
| 503 | AACUACUCCCAGUACCAAA |
| 504 | ACUACUCCCAGUACCAAAU |
| 505 | CUACUCCCAGUACCAAAUG |
| 506 | UACUCCCAGUACCAAAUGG |
| 507 | ACUCCCAGUACCAAAUGGC |
| 508 | CUCCCAGUACCAAAUGGCA |
| 509 | UCCCAGUACCAAAUGGCAG |
| 510 | CCCAGUACCAAAUGGCAGU |
| 511 | CCAGUACCAAAUGGCAGUG |
| 512 | CAGUACCAAAUGGCAGUGG |
| 513 | AGUACCAAAUGGCAGUGGC |
| 514 | GUACCAAAUGGCAGUGGCC |
| 515 | UACCAAAUGGCAGUGGCCA |
| 516 | ACCAAAUGGCAGUGGCCAC |
| 517 | CCAAAUGGCAGUGGCCACU |
| 518 | CAAAUGGCAGUGGCCACUG |
| 519 | AAAUGGCAGUGGCCACUGA |
| 520 | AAUGGCAGUGGCCACUGAG |
| 521 | AUGGCAGUGGCCACUGAGU |
| 522 | UGGCAGUGGCCACUGAGUC |
| 523 | GGCAGUGGCCACUGAGUCU |
| 524 | GCAGUGGCCACUGAGUCUU |
| 525 | CAGUGGCCACUGAGUCUUC |
| 526 | AGUGGCCACUGAGUCUUCC |
| 527 | GUGGCCACUGAGUCUUCCU |
| 528 | UGGCCACUGAGUCUUCCUC |
| 529 | GGCCACUGAGUCUUCCUCA |
| 530 | GCCACUGAGUCUUCCUCAA |
| 531 | CCACUGAGUCUUCCUCAAG |
| 532 | CACUGAGUCUUCCUCAAGU |
| 533 | ACUGAGUCUUCCUCAAGUG |
| 534 | CUGAGUCUUCCUCAAGUGA |
| 535 | UGAGUCUUCCUCAAGUGAG |
| 536 | GAGUCUUCCUCAAGUGAGA |
| 537 | AGUCUUCCUCAAGUGAGAC |
| 538 | GUCUUCCUCAAGUGAGACA |
| 539 | UCUUCCUCAAGUGAGACAG |
| 540 | CUUCCUCAAGUGAGACAGG |
| 541 | UUCCUCAAGUGAGACAGGG |
| 542 | UCCUCAAGUGAGACAGGGG |
| 543 | CCUCAAGUGAGACAGGGGG |
| 544 | CUCAAGUGAGACAGGGGGU |
| 545 | UCAAGUGAGACAGGGGGUA |
| 546 | CAAGUGAGACAGGGGGUAC |
| 547 | AAGUGAGACAGGGGGUACG |
| 548 | AGUGAGACAGGGGGUACGU |
| 549 | GUGAGACAGGGGGUACGUU |
| 550 | UGAGACAGGGGGUACGUUU |
| 551 | GAGACAGGGGGUACGUUUG |
| 552 | AGACAGGGGGUACGUUUGU |
| 553 | GACAGGGGGUACGUUUGUA |
| 554 | ACAGGGGGUACGUUUGUAG |
| 555 | CAGGGGGUACGUUUGUAGG |
| 556 | AGGGGGUACGUUUGUAGGG |
| 557 | GGGGGUACGUUUGUAGGGU |
| 558 | GGGGUACGUUUGUAGGGUC |
| 559 | GGGUACGUUUGUAGGGUCA |
| 560 | GGUACGUUUGUAGGGUCAG |
| 561 | GUACGUUUGUAGGGUCAGC |
| 562 | UACGUUUGUAGGGUCAGCC |
| 563 | ACGUUUGUAGGGUCAGCCA |
| 564 | CGUUUGUAGGGUCAGCCAU |
| 565 | GUUUGUAGGGUCAGCCAUG |
| 566 | UUUGUAGGGUCAGCCAUGA |
| 567 | UUGUAGGGUCAGCCAUGAA |
| 568 | UGUAGGGUCAGCCAUGAAA |
| 569 | GUAGGGUCAGCCAUGAAAA |
| 570 | UAGGGUCAGCCAUGAAAAA |
| 571 | AGGGUCAGCCAUGAAAAAC |
| 572 | GGGUCAGCCAUGAAAAACA |
| 573 | GGUCAGCCAUGAAAAACAG |
| 574 | GUCAGCCAUGAAAAACAGC |
| 575 | UCAGCCAUGAAAAACAGCC |
| 576 | CAGCCAUGAAAAACAGCCU |
| 577 | AGCCAUGAAAAACAGCCUU |
| 578 | GCCAUGAAAAACAGCCUUC |
| 579 | CCAUGAAAAACAGCCUUCG |
| 580 | CAUGAAAAACAGCCUUCGA |
| 581 | AUGAAAAACAGCCUUCGAA |
| 582 | UGAAAAACAGCCUUCGAAG |
| 583 | GAAAAACAGCCUUCGAAGC |
| 584 | AAAAACAGCCUUCGAAGCC |
| 585 | AAAACAGCCUUCGAAGCCU |
| 586 | AAACAGCCUUCGAAGCCUC |
| 587 | AACAGCCUUCGAAGCCUCC |
| 588 | ACAGCCUUCGAAGCCUCCC |
| 589 | CAGCCUUCGAAGCCUCCCA |
| 590 | AGCCUUCGAAGCCUCCCAG |
| 591 | GCCUUCGAAGCCUCCCAGC |
| 592 | CCUUCGAAGCCUCCCAGCA |
| 593 | CUUCGAAGCCUCCCAGCAA |
| 594 | UUCGAAGCCUCCCAGCAAC |
| 595 | UCGAAGCCUCCCAGCAACA |
| 596 | CGAAGCCUCCCAGCAACAU |
| 597 | GAAGCCUCCCAGCAACAUA |
| 598 | AAGCCUCCCAGCAACAUAC |
| 599 | AGCCUCCCAGCAACAUACA |
| 600 | GCCUCCCAGCAACAUACAU |
| 601 | CCUCCCAGCAACAUACAUG |
| 602 | CUCCCAGCAACAUACAUGU |
| 603 | UCCCAGCAACAUACAUGUC |
| 604 | CCCAGCAACAUACAUGUCA |
| 605 | CCAGCAACAUACAUGUCAA |
| 606 | CAGCAACAUACAUGUCAAG |
| 607 | AGCAACAUACAUGUCAAGC |
| 608 | GCAACAUACAUGUCAAGCC |
| 609 | CAACAUACAUGUCAAGCCA |
| 610 | AACAUACAUGUCAAGCCAG |
| 611 | ACAUACAUGUCAAGCCAGU |
| 612 | CAUACAUGUCAAGCCAGUC |
| 613 | AUACAUGUCAAGCCAGUCA |
| 614 | UACAUGUCAAGCCAGUCAG |
| 615 | ACAUGUCAAGCCAGUCAGG |
| 616 | CAUGUCAAGCCAGUCAGGA |
| 617 | AUGUCAAGCCAGUCAGGAA |
| 618 | UGUCAAGCCAGUCAGGAAA |
| 619 | GUCAAGCCAGUCAGGAAAA |
| 620 | UCAAGCCAGUCAGGAAAAC |
| 621 | CAAGCCAGUCAGGAAAACA |
| 622 | AAGCCAGUCAGGAAAACAG |
| 623 | AGCCAGUCAGGAAAACAGU |
| 624 | GCCAGUCAGGAAAACAGUG |
| 625 | CCAGUCAGGAAAACAGUGG |
| 626 | CAGUCAGGAAAACAGUGGC |
| 627 | AGUCAGGAAAACAGUGGCA |
| 628 | GUCAGGAAAACAGUGGCAG |
| 629 | UCAGGAAAACAGUGGCAGA |
| 630 | CAGGAAAACAGUGGCAGAU |
| 631 | AGGAAAACAGUGGCAGAUG |
| 632 | GGAAAACAGUGGCAGAUGA |
| 633 | GAAAACAGUGGCAGAUGAA |
| 634 | AAAACAGUGGCAGAUGAAG |
| 635 | AAACAGUGGCAGAUGAAGG |
| 636 | AACAGUGGCAGAUGAAGGG |
| 637 | ACAGUGGCAGAUGAAGGGA |
| 638 | CAGUGGCAGAUGAAGGGAA |
| 639 | AGUGGCAGAUGAAGGGAAU |
| 640 | GUGGCAGAUGAAGGGAAUG |
| 641 | UGGCAGAUGAAGGGAAUGG |
| 642 | GGCAGAUGAAGGGAAUGGA |
| 643 | GCAGAUGAAGGGAAUGGAG |
| 644 | CAGAUGAAGGGAAUGGAGA |
| 645 | AGAUGAAGGGAAUGGAGAA |
| 646 | GAUGAAGGGAAUGGAGAAC |
| 647 | AUGAAGGGAAUGGAGAACC |
| 648 | UGAAGGGAAUGGAGAACCG |
| 649 | GAAGGGAAUGGAGAACCGC |
| 650 | AAGGGAAUGGAGAACCGCC |
| 651 | AGGGAAUGGAGAACCGCCA |
| 652 | GGGAAUGGAGAACCGCCAU |
| 653 | GGAAUGGAGAACCGCCAUG |
| 654 | GAAUGGAGAACCGCCAUGC |
| 655 | AAUGGAGAACCGCCAUGCC |
| 656 | AUGGAGAACCGCCAUGCCA |
| 657 | UGGAGAACCGCCAUGCCAU |
| 658 | GGAGAACCGCCAUGCCAUG |
| 659 | GAGAACCGCCAUGCCAUGA |
| 660 | AGAACCGCCAUGCCAUGAG |
| 661 | GAACCGCCAUGCCAUGAGC |
| 662 | AACCGCCAUGCCAUGAGCU |
| 663 | ACCGCCAUGCCAUGAGCUC |
| 664 | CCGCCAUGCCAUGAGCUCC |
| 665 | CGCCAUGCCAUGAGCUCCC |
| 666 | GCCAUGCCAUGAGCUCCCA |
| 667 | CCAUGCCAUGAGCUCCCAG |
| 668 | CAUGCCAUGAGCUCCCAGU |
| 669 | AUGCCAUGAGCUCCCAGUA |
| 670 | UGCCAUGAGCUCCCAGUAC |
| 671 | GCCAUGAGCUCCCAGUACC |
| 672 | CCAUGAGCUCCCAGUACCG |
| 673 | CAUGAGCUCCCAGUACCGG |
| 674 | AUGAGCUCCCAGUACCGGA |
| 675 | UGAGCUCCCAGUACCGGAU |
| 676 | GAGCUCCCAGUACCGGAUG |
| 677 | AGCUCCCAGUACCGGAUGU |
| 678 | GCUCCCAGUACCGGAUGUG |
| 679 | CUCCCAGUACCGGAUGUGC |
| 680 | UCCCAGUACCGGAUGUGCU |
| 681 | CCCAGUACCGGAUGUGCUC |
| 682 | CCAGUACCGGAUGUGCUCC |
| 683 | CAGUACCGGAUGUGCUCCU |
| 684 | AGUACCGGAUGUGCUCCUA |
| 685 | GUACCGGAUGUGCUCCUAC |
| 686 | UACCGGAUGUGCUCCUACU |
| 687 | ACCGGAUGUGCUCCUACUA |
| 688 | CCGGAUGUGCUCCUACUAC |
| 689 | CGGAUGUGCUCCUACUACC |
| 690 | GGAUGUGCUCCUACUACCC |
| 691 | GAUGUGCUCCUACUACCCG |
| 692 | AUGUGCUCCUACUACCCGC |
| 693 | UGUGCUCCUACUACCCGCC |
| 694 | GUGCUCCUACUACCCGCCC |
| 695 | UGCUCCUACUACCCGCCCA |
| 696 | GCUCCUACUACCCGCCCAC |
| 697 | CUCCUACUACCCGCCCACC |
| 698 | UCCUACUACCCGCCCACCU |
| 699 | CCUACUACCCGCCCACCUC |
| 700 | CUACUACCCGCCCACCUCA |
| 701 | UACUACCCGCCCACCUCAU |
| 702 | ACUACCCGCCCACCUCAUA |
| 703 | CUACCCGCCCACCUCAUAC |
| 704 | UACCCGCCCACCUCAUACC |
| 705 | ACCCGCCCACCUCAUACCU |
| 706 | CCCGCCCACCUCAUACCUG |
| 707 | CCGCCCACCUCAUACCUGG |
| 708 | CGCCCACCUCAUACCUGGG |
| 709 | GCCCACCUCAUACCUGGGC |
| 710 | CCCACCUCAUACCUGGGCC |
| 711 | CCACCUCAUACCUGGGCCA |
| 712 | CACCUCAUACCUGGGCCAG |
| 713 | ACCUCAUACCUGGGCCAGG |
| 714 | CCUCAUACCUGGGCCAGGG |
| 715 | CUCAUACCUGGGCCAGGGG |
| 716 | UCAUACCUGGGCCAGGGGG |
| 717 | CAUACCUGGGCCAGGGGGU |
| 718 | AUACCUGGGCCAGGGGGUU |
| 719 | UACCUGGGCCAGGGGGUUG |
| 720 | ACCUGGGCCAGGGGGUUGG |
| 721 | CCUGGGCCAGGGGGUUGGC |
| 722 | CUGGGCCAGGGGGUUGGCA |
| 723 | UGGGCCAGGGGGUUGGCAG |
| 724 | GGGCCAGGGGGUUGGCAGU |
| 725 | GGCCAGGGGGUUGGCAGUC |
| 726 | GCCAGGGGGUUGGCAGUCC |
| 727 | CCAGGGGGUUGGCAGUCCC |
| 728 | CAGGGGGUUGGCAGUCCCA |
| 729 | AGGGGGUUGGCAGUCCCAC |
| 730 | GGGGGUUGGCAGUCCCACC |
| 731 | GGGGUUGGCAGUCCCACCU |
| 732 | GGGUUGGCAGUCCCACCUG |
| 733 | GGUUGGCAGUCCCACCUGC |
| 734 | GUUGGCAGUCCCACCUGCG |
| 735 | UUGGCAGUCCCACCUGCGU |
| 736 | UGGCAGUCCCACCUGCGUC |
| 737 | GGCAGUCCCACCUGCGUCA |
| 738 | GCAGUCCCACCUGCGUCAC |
| 739 | CAGUCCCACCUGCGUCACA |
| 740 | AGUCCCACCUGCGUCACAC |
| 741 | GUCCCACCUGCGUCACACA |
| 742 | UCCCACCUGCGUCACACAG |
| 743 | CCCACCUGCGUCACACAGA |
| 744 | CCACCUGCGUCACACAGAU |
| 745 | CACCUGCGUCACACAGAUA |
| 746 | ACCUGCGUCACACAGAUAC |
| 747 | CCUGCGUCACACAGAUACU |
| 748 | CUGCGUCACACAGAUACUG |
| 749 | UGCGUCACACAGAUACUGG |
| 750 | GCGUCACACAGAUACUGGC |
| 751 | CGUCACACAGAUACUGGCC |
| 752 | GUCACACAGAUACUGGCCU |
| 753 | UCACACAGAUACUGGCCUC |
| 754 | CACACAGAUACUGGCCUCG |
| 755 | ACACAGAUACUGGCCUCGG |
| 756 | CACAGAUACUGGCCUCGGA |
| 757 | ACAGAUACUGGCCUCGGAG |
| 758 | CAGAUACUGGCCUCGGAGG |
| 759 | AGAUACUGGCCUCGGAGGA |
| 760 | GAUACUGGCCUCGGAGGAC |
| 761 | AUACUGGCCUCGGAGGACA |
| 762 | UACUGGCCUCGGAGGACAC |
| 763 | ACUGGCCUCGGAGGACACC |
| 764 | CUGGCCUCGGAGGACACCC |
| 765 | UGGCCUCGGAGGACACCCC |
| 766 | GGCCUCGGAGGACACCCCC |
| 767 | GCCUCGGAGGACACCCCCU |
| 768 | CCUCGGAGGACACCCCCUC |
| 769 | CUCGGAGGACACCCCCUCC |
| 770 | UCGGAGGACACCCCCUCCU |
| 771 | CGGAGGACACCCCCUCCUA |
| 772 | GGAGGACACCCCCUCCUAC |
| 773 | GAGGACACCCCCUCCUACU |
| 774 | AGGACACCCCCUCCUACUC |
| 775 | GGACACCCCCUCCUACUCA |
| 776 | GACACCCCCUCCUACUCAG |
| 777 | ACACCCCCUCCUACUCAGA |
| 778 | CACCCCCUCCUACUCAGAG |
| 779 | ACCCCCUCCUACUCAGAGU |
| 780 | CCCCCUCCUACUCAGAGUC |
| 781 | CCCCUCCUACUCAGAGUCG |
| 782 | CCCUCCUACUCAGAGUCGA |
| 783 | CCUCCUACUCAGAGUCGAA |
| 784 | CUCCUACUCAGAGUCGAAA |
| 785 | UCCUACUCAGAGUCGAAAG |
| 786 | CCUACUCAGAGUCGAAAGC |
| 787 | CUACUCAGAGUCGAAAGCG |
| 788 | UACUCAGAGUCGAAAGCGA |
| 789 | ACUCAGAGUCGAAAGCGAG |
| 790 | CUCAGAGUCGAAAGCGAGA |
| 791 | UCAGAGUCGAAAGCGAGAG |
| 792 | CAGAGUCGAAAGCGAGAGU |
| 793 | AGAGUCGAAAGCGAGAGUG |
| 794 | GAGUCGAAAGCGAGAGUGU |
| 795 | AGUCGAAAGCGAGAGUGUU |
| 796 | GUCGAAAGCGAGAGUGUUU |
| 797 | UCGAAAGCGAGAGUGUUUU |
| 798 | CGAAAGCGAGAGUGUUUUC |
| 799 | GAAAGCGAGAGUGUUUUCG |
| 800 | AAAGCGAGAGUGUUUUCGC |
| 801 | AAGCGAGAGUGUUUUCGCC |
| 802 | AGCGAGAGUGUUUUCGCCG |
| 803 | GCGAGAGUGUUUUCGCCGC |
| 804 | CGAGAGUGUUUUCGCCGCC |
| 805 | GAGAGUGUUUUCGCCGCCC |
| 806 | AGAGUGUUUUCGCCGCCCA |
| 807 | GAGUGUUUUCGCCGCCCAG |
| 808 | AGUGUUUUCGCCGCCCAGC |
| 809 | GUGUUUUCGCCGCCCAGCA |
| 810 | UGUUUUCGCCGCCCAGCAG |
| 811 | GUUUUCGCCGCCCAGCAGC |
| 812 | UUUUCGCCGCCCAGCAGCC |
| 813 | UUUCGCCGCCCAGCAGCCA |
| 814 | UUCGCCGCCCAGCAGCCAG |
| 815 | UCGCCGCCCAGCAGCCAGG |
| 816 | CGCCGCCCAGCAGCCAGGA |
| 817 | GCCGCCCAGCAGCCAGGAC |
| 818 | CCGCCCAGCAGCCAGGACU |
| 819 | CGCCCAGCAGCCAGGACUC |
| 820 | GCCCAGCAGCCAGGACUCG |
| 821 | CCCAGCAGCCAGGACUCGG |
| 822 | CCAGCAGCCAGGACUCGGG |
| 823 | CAGCAGCCAGGACUCGGGC |
| 824 | AGCAGCCAGGACUCGGGCC |
| 825 | GCAGCCAGGACUCGGGCCU |
| 826 | CAGCCAGGACUCGGGCCUG |
| 827 | AGCCAGGACUCGGGCCUGG |
| 828 | GCCAGGACUCGGGCCUGGG |
| 829 | CCAGGACUCGGGCCUGGGG |
| 830 | CAGGACUCGGGCCUGGGGU |
| 831 | AGGACUCGGGCCUGGGGUG |
| 832 | GGACUCGGGCCUGGGGUGC |
| 833 | GACUCGGGCCUGGGGUGCC |
| 834 | ACUCGGGCCUGGGGUGCCU |
| 835 | CUCGGGCCUGGGGUGCCUG |
| 836 | UCGGGCCUGGGGUGCCUGU |
| 837 | CGGGCCUGGGGUGCCUGUC |
| 838 | GGGCCUGGGGUGCCUGUCG |
| 839 | GGCCUGGGGUGCCUGUCGA |
| 840 | GCCUGGGGUGCCUGUCGAG |
| 841 | CCUGGGGUGCCUGUCGAGC |
| 842 | CUGGGGUGCCUGUCGAGCA |
| 843 | UGGGGUGCCUGUCGAGCAG |
| 844 | GGGGUGCCUGUCGAGCAGC |
| 845 | GGGUGCCUGUCGAGCAGCG |
| 846 | GGUGCCUGUCGAGCAGCGA |
| 847 | GUGCCUGUCGAGCAGCGAG |
| 848 | UGCCUGUCGAGCAGCGAGA |
| 849 | GCCUGUCGAGCAGCGAGAG |
| 850 | CCUGUCGAGCAGCGAGAGC |
| 851 | CUGUCGAGCAGCGAGAGCA |
| 852 | UGUCGAGCAGCGAGAGCAC |
| 853 | GUCGAGCAGCGAGAGCACC |
| 854 | UCGAGCAGCGAGAGCACCA |
| 855 | CGAGCAGCGAGAGCACCAA |
| 856 | GAGCAGCGAGAGCACCAAG |
| 857 | AGCAGCGAGAGCACCAAGG |
| 858 | GCAGCGAGAGCACCAAGGG |
| 859 | CAGCGAGAGCACCAAGGGA |
| 860 | AGCGAGAGCACCAAGGGAG |
| 861 | GCGAGAGCACCAAGGGAGA |
| 862 | CGAGAGCACCAAGGGAGAC |
| 863 | GAGAGCACCAAGGGAGACC |
| 864 | AGAGCACCAAGGGAGACCU |
| 865 | GAGCACCAAGGGAGACCUG |
| 866 | AGCACCAAGGGAGACCUGG |
| 867 | GCACCAAGGGAGACCUGGA |
| 868 | CACCAAGGGAGACCUGGAG |
| 869 | ACCAAGGGAGACCUGGAGU |
| 870 | CCAAGGGAGACCUGGAGUG |
| 871 | CAAGGGAGACCUGGAGUGC |
| 872 | AAGGGAGACCUGGAGUGCG |
| 873 | AGGGAGACCUGGAGUGCGA |
| 874 | GGGAGACCUGGAGUGCGAG |
| 875 | GGAGACCUGGAGUGCGAGC |
| 876 | GAGACCUGGAGUGCGAGCC |
| 877 | AGACCUGGAGUGCGAGCCC |
| 878 | GACCUGGAGUGCGAGCCCC |
| 879 | ACCUGGAGUGCGAGCCCCA |
| 880 | CCUGGAGUGCGAGCCCCAC |
| 881 | CUGGAGUGCGAGCCCCACC |
| 882 | UGGAGUGCGAGCCCCACCA |
| 883 | GGAGUGCGAGCCCCACCAA |
| 884 | GAGUGCGAGCCCCACCAAG |
| 885 | AGUGCGAGCCCCACCAAGA |
| 886 | GUGCGAGCCCCACCAAGAG |
| 887 | UGCGAGCCCCACCAAGAGC |
| 888 | GCGAGCCCCACCAAGAGCC |
| 889 | CGAGCCCCACCAAGAGCCC |
| 890 | GAGCCCCACCAAGAGCCCG |
| 891 | AGCCCCACCAAGAGCCCGG |
| 892 | GCCCCACCAAGAGCCCGGC |
| 893 | CCCCACCAAGAGCCCGGCG |
| 894 | CCCACCAAGAGCCCGGCGC |
| 895 | CCACCAAGAGCCCGGCGCC |
| 896 | CACCAAGAGCCCGGCGCCU |
| 897 | ACCAAGAGCCCGGCGCCUU |
| 898 | CCAAGAGCCCGGCGCCUUC |
| 899 | CAAGAGCCCGGCGCCUUCG |
| 900 | AAGAGCCCGGCGCCUUCGC |
| 901 | AGAGCCCGGCGCCUUCGCG |
| 902 | GAGCCCGGCGCCUUCGCGG |
| 903 | AGCCCGGCGCCUUCGCGGU |
| 904 | GCCCGGCGCCUUCGCGGUG |
| 905 | CCCGGCGCCUUCGCGGUGA |
| 906 | CCGGCGCCUUCGCGGUGAG |
| 907 | CGGCGCCUUCGCGGUGAGC |
| 908 | GGCGCCUUCGCGGUGAGCC |
| 909 | GCGCCUUCGCGGUGAGCCC |
| 910 | CGCCUUCGCGGUGAGCCCG |
| 911 | GCCUUCGCGGUGAGCCCGG |
| 912 | CCUUCGCGGUGAGCCCGGU |
| 913 | CUUCGCGGUGAGCCCGGUU |
| 914 | UUCGCGGUGAGCCCGGUUC |
| 915 | UCGCGGUGAGCCCGGUUCU |
| 916 | CGCGGUGAGCCCGGUUCUU |
| 917 | GCGGUGAGCCCGGUUCUUG |
| 918 | CGGUGAGCCCGGUUCUUGA |
| 919 | GGUGAGCCCGGUUCUUGAG |
| 920 | GUGAGCCCGGUUCUUGAGG |
| 921 | UGAGCCCGGUUCUUGAGGG |
| 922 | GAGCCCGGUUCUUGAGGGC |
| 923 | AGCCCGGUUCUUGAGGGCG |
| 924 | GCCCGGUUCUUGAGGGCGA |
| 925 | CCCGGUUCUUGAGGGCGAG |
| 926 | CCGGUUCUUGAGGGCGAGU |
| 927 | CGGUUCUUGAGGGCGAGUA |
| 928 | GGUUCUUGAGGGCGAGUAG |
| 929 | GUUCUUGAGGGCGAGUAGG |
| 930 | UUCUUGAGGGCGAGUAGGC |
| 931 | UCUUGAGGGCGAGUAGGCG |
| 932 | CUUGAGGGCGAGUAGGCGC |
| 933 | UUGAGGGCGAGUAGGCGCG |
| 934 | UGAGGGCGAGUAGGCGCGG |
| 935 | GAGGGCGAGUAGGCGCGGC |
| 936 | AGGGCGAGUAGGCGCGGCG |
| 937 | GGGCGAGUAGGCGCGGCGU |
| 938 | GGCGAGUAGGCGCGGCGUC |
| 939 | GCGAGUAGGCGCGGCGUCG |
| 940 | CGAGUAGGCGCGGCGUCGG |
| 941 | GAGUAGGCGCGGCGUCGGG |
| 942 | AGUAGGCGCGGCGUCGGGC |
| 943 | GUAGGCGCGGCGUCGGGCG |
| 944 | UAGGCGCGGCGUCGGGCGG |
| 945 | AGGCGCGGCGUCGGGCGGC |
| 946 | GGCGCGGCGUCGGGCGGCU |
| 947 | GCGCGGCGUCGGGCGGCUG |
| 948 | CGCGGCGUCGGGCGGCUGC |
| 949 | GCGGCGUCGGGCGGCUGCU |
| 950 | CGGCGUCGGGCGGCUGCUG |
| 951 | GGCGUCGGGCGGCUGCUGC |
| 952 | GCGUCGGGCGGCUGCUGCG |
| 953 | CGUCGGGCGGCUGCUGCGC |
| 954 | GUCGGGCGGCUGCUGCGCG |
| 955 | UCGGGCGGCUGCUGCGCGG |
| 956 | CGGGCGGCUGCUGCGCGGC |
| 957 | GGGCGGCUGCUGCGCGGCG |
| 958 | GGCGGCUGCUGCGCGGCGU |
| 959 | GCGGCUGCUGCGCGGCGUU |
| 960 | CGGCUGCUGCGCGGCGUUC |
| 961 | GGCUGCUGCGCGGCGUUCA |
| 962 | GCUGCUGCGCGGCGUUCAC |
| 963 | CUGCUGCGCGGCGUUCACU |
| 964 | UGCUGCGCGGCGUUCACUG |
| 965 | GCUGCGCGGCGUUCACUGU |
| 966 | CUGCGCGGCGUUCACUGUU |
| 967 | UGCGCGGCGUUCACUGUUG |
| 968 | GCGCGGCGUUCACUGUUGC |
| 969 | CGCGGCGUUCACUGUUGCC |
| 970 | GCGGCGUUCACUGUUGCCU |
| 971 | CGGCGUUCACUGUUGCCUU |
| 972 | GGCGUUCACUGUUGCCUUG |
| 973 | GCGUUCACUGUUGCCUUGU |
| 974 | CGUUCACUGUUGCCUUGUU |
| 975 | GUUCACUGUUGCCUUGUUC |
| 976 | UUCACUGUUGCCUUGUUCU |
| 977 | UCACUGUUGCCUUGUUCUG |
| 978 | CACUGUUGCCUUGUUCUGU |
| 979 | ACUGUUGCCUUGUUCUGUU |
| 980 | CUGUUGCCUUGUUCUGUUG |
| 981 | UGUUGCCUUGUUCUGUUGG |
| 982 | GUUGCCUUGUUCUGUUGGG |
| 983 | UUGCCUUGUUCUGUUGGGG |
| 984 | UGCCUUGUUCUGUUGGGGU |
| 985 | GCCUUGUUCUGUUGGGGUU |
| 986 | CCUUGUUCUGUUGGGGUUG |
| 987 | CUUGUUCUGUUGGGGUUGC |
| 988 | UUGUUCUGUUGGGGUUGCG |
| 989 | UGUUCUGUUGGGGUUGCGG |
| 990 | GUUCUGUUGGGGUUGCGGG |
| 991 | UUCUGUUGGGGUUGCGGGG |
| 992 | UCUGUUGGGGUUGCGGGGG |
| 993 | CUGUUGGGGUUGCGGGGGG |
| 994 | UGUUGGGGUUGCGGGGGGG |
| 995 | GUUGGGGUUGCGGGGGGGC |
| 996 | UUGGGGUUGCGGGGGGGCG |
| 997 | UGGGGUUGCGGGGGGGCGU |
| 998 | GGGGUUGCGGGGGGGCGUU |
| 999 | GGGUUGCGGGGGGGCGUUG |
| 1000 | GGUUGCGGGGGGGCGUUGG |
| 1001 | GUUGCGGGGGGGCGUUGGG |
| 1002 | UUGCGGGGGGGCGUUGGGU |
| 1003 | UGCGGGGGGGCGUUGGGUU |
| 1004 | GCGGGGGGGCGUUGGGUUU |
| 1005 | CGGGGGGGCGUUGGGUUUC |
| 1006 | GGGGGGGCGUUGGGUUUCU |
| 1007 | GGGGGGCGUUGGGUUUCUU |
| 1008 | GGGGGCGUUGGGUUUCUUC |
| 1009 | GGGGCGUUGGGUUUCUUCU |
| 1010 | GGGCGUUGGGUUUCUUCUU |
| 1011 | GGCGUUGGGUUUCUUCUUU |
| 1012 | GCGUUGGGUUUCUUCUUUC |
| 1013 | CGUUGGGUUUCUUCUUUCC |
| 1014 | GUUGGGUUUCUUCUUUCCG |
| 1015 | UUGGGUUUCUUCUUUCCGG |
| 1016 | UGGGUUUCUUCUUUCCGGG |
| 1017 | GGGUUUCUUCUUUCCGGGG |
| 1018 | GGUUUCUUCUUUCCGGGGC |
| 1019 | GUUUCUUCUUUCCGGGGCG |
| 1020 | UUUCUUCUUUCCGGGGCGG |
| 1021 | UUCUUCUUUCCGGGGCGGG |
| 1022 | UCUUCUUUCCGGGGCGGGG |
| 1023 | CUUCUUUCCGGGGCGGGGG |
| 1024 | UUCUUUCCGGGGCGGGGGG |
| 1025 | UCUUUCCGGGGCGGGGGGG |
| 1026 | CUUUCCGGGGCGGGGGGGG |
| 1027 | UUUCCGGGGCGGGGGGGGC |
| 1028 | UUCCGGGGCGGGGGGGGCA |
| 1029 | UCCGGGGCGGGGGGGGCAC |
| 1030 | CCGGGGCGGGGGGGGCACG |
| 1031 | CGGGGCGGGGGGGGCACGG |
| 1032 | GGGGCGGGGGGGGCACGGC |
| 1033 | GGGCGGGGGGGGCACGGCG |
| 1034 | GGCGGGGGGGGCACGGCGG |
| 1035 | GCGGGGGGGGCACGGCGGG |
| 1036 | CGGGGGGGGCACGGCGGGG |
| 1037 | GGGGGGGGCACGGCGGGGC |
| 1038 | GGGGGGGCACGGCGGGGCC |
| 1039 | GGGGGGCACGGCGGGGCCG |
| 1040 | GGGGGCACGGCGGGGCCGC |
| 1041 | GGGGCACGGCGGGGCCGCG |
| 1042 | GGGCACGGCGGGGCCGCGG |
| 1043 | GGCACGGCGGGGCCGCGGC |
| 1044 | GCACGGCGGGGCCGCGGCC |
| 1045 | CACGGCGGGGCCGCGGCCG |
| 1046 | ACGGCGGGGCCGCGGCCGG |
| 1047 | CGGCGGGGCCGCGGCCGGG |
| 1048 | GGCGGGGCCGCGGCCGGGC |
| 1049 | GCGGGGCCGCGGCCGGGCC |
| 1050 | CGGGGCCGCGGCCGGGCCG |
| 1051 | GGGGCCGCGGCCGGGCCGG |
| 1052 | GGGCCGCGGCCGGGCCGGC |
| 1053 | GGCCGCGGCCGGGCCGGCG |
| 1054 | GCCGCGGCCGGGCCGGCGG |
| 1055 | CCGCGGCCGGGCCGGCGGG |
| 1056 | CGCGGCCGGGCCGGCGGGG |
| 1057 | GCGGCCGGGCCGGCGGGGC |
| 1058 | CGGCCGGGCCGGCGGGGCG |
| 1059 | GGCCGGGCCGGCGGGGCGG |
| 1060 | GCCGGGCCGGCGGGGCGGG |
| 1061 | CCGGGCCGGCGGGGCGGGG |
| 1062 | CGGGCCGGCGGGGCGGGGC |
| 1063 | GGGCCGGCGGGGCGGGGCG |
| 1064 | GGCCGGCGGGGCGGGGCGG |
| 1065 | GCCGGCGGGGCGGGGCGGG |
| 1066 | CCGGCGGGGCGGGGCGGGG |
| 1067 | CGGCGGGGCGGGGCGGGGC |
| 1068 | GGCGGGGCGGGGCGGGGCG |
| 1069 | GCGGGGCGGGGCGGGGCGG |
| 1070 | CGGGGCGGGGCGGGGCGGG |
| 1071 | GGGGCGGGGCGGGGCGGGA |
| 1072 | GGGCGGGGCGGGGCGGGAC |
| 1073 | GGCGGGGCGGGGCGGGACG |
| 1074 | GCGGGGCGGGGCGGGACGG |
| 1075 | CGGGGCGGGGCGGGACGGG |
| 1076 | GGGGCGGGGCGGGACGGGG |
| 1077 | GGGCGGGGCGGGACGGGGC |
| 1078 | GGCGGGGCGGGACGGGGCG |
| 1079 | GCGGGGCGGGACGGGGCGG |
| 1080 | CGGGGCGGGACGGGGCGGG |
| 1081 | GGGGCGGGACGGGGCGGGG |
| 1082 | GGGCGGGACGGGGCGGGGC |
| 1083 | GGCGGGACGGGGCGGGGCG |
| 1084 | GCGGGACGGGGCGGGGCGG |
| 1085 | CGGGACGGGGCGGGGCGGA |
| 1086 | GGGACGGGGCGGGGCGGAG |
| 1087 | GGACGGGGCGGGGCGGAGC |
| 1088 | GACGGGGCGGGGCGGAGCC |
| 1089 | ACGGGGCGGGGCGGAGCCG |
| 1090 | CGGGGCGGGGCGGAGCCGC |
| 1091 | GGGGCGGGGCGGAGCCGCG |
| 1092 | GGGCGGGGCGGAGCCGCGC |
| 1093 | GGCGGGGCGGAGCCGCGCG |
| 1094 | GCGGGGCGGAGCCGCGCGG |
| 1095 | CGGGGCGGAGCCGCGCGGG |
| 1096 | GGGGCGGAGCCGCGCGGGG |
| 1097 | GGGCGGAGCCGCGCGGGGG |
| 1098 | GGCGGAGCCGCGCGGGGGC |
| 1099 | GCGGAGCCGCGCGGGGGCC |
| 1100 | CGGAGCCGCGCGGGGGCCG |
| 1101 | GGAGCCGCGCGGGGGCCGC |
| 1102 | GAGCCGCGCGGGGGCCGCA |
| 1103 | AGCCGCGCGGGGGCCGCAG |
| 1104 | GCCGCGCGGGGGCCGCAGU |
| 1105 | CCGCGCGGGGGCCGCAGUC |
| 1106 | CGCGCGGGGGCCGCAGUCC |
| 1107 | GCGCGGGGGCCGCAGUCCG |
| 1108 | CGCGGGGGCCGCAGUCCGG |
| 1109 | GCGGGGGCCGCAGUCCGGG |
| 1110 | CGGGGGCCGCAGUCCGGGC |
| 1111 | GGGGGCCGCAGUCCGGGCC |
| 1112 | GGGGCCGCAGUCCGGGCCG |
| 1113 | GGGCCGCAGUCCGGGCCGG |
| 1114 | GGCCGCAGUCCGGGCCGGG |
| 1115 | GCCGCAGUCCGGGCCGGGG |
| 1116 | CCGCAGUCCGGGCCGGGGC |
| 1117 | CGCAGUCCGGGCCGGGGCC |
| 1118 | GCAGUCCGGGCCGGGGCCG |
| 1119 | CAGUCCGGGCCGGGGCCGC |
| 1120 | AGUCCGGGCCGGGGCCGCC |
| 1121 | GUCCGGGCCGGGGCCGCCG |
| 1122 | UCCGGGCCGGGGCCGCCGU |
| 1123 | CCGGGCCGGGGCCGCCGUC |
| 1124 | CGGGCCGGGGCCGCCGUCG |
| 1125 | GGGCCGGGGCCGCCGUCGG |
| 1126 | GGCCGGGGCCGCCGUCGGG |
| 1127 | GCCGGGGCCGCCGUCGGGU |
| 1128 | CCGGGGCCGCCGUCGGGUC |
| 1129 | CGGGGCCGCCGUCGGGUCU |
| 1130 | GGGGCCGCCGUCGGGUCUC |
| 1131 | GGGCCGCCGUCGGGUCUCG |
| 1132 | GGCCGCCGUCGGGUCUCGG |
| 1133 | GCCGCCGUCGGGUCUCGGC |
| 1134 | CCGCCGUCGGGUCUCGGCC |
| 1135 | CGCCGUCGGGUCUCGGCCC |
| 1136 | GCCGUCGGGUCUCGGCCCG |
| 1137 | CCGUCGGGUCUCGGCCCGC |
| 1138 | CGUCGGGUCUCGGCCCGCU |
| 1139 | GUCGGGUCUCGGCCCGCUC |
| 1140 | UCGGGUCUCGGCCCGCUCC |
| 1141 | CGGGUCUCGGCCCGCUCCC |
| 1142 | GGGUCUCGGCCCGCUCCCG |
| 1143 | GGUCUCGGCCCGCUCCCGU |
| 1144 | GUCUCGGCCCGCUCCCGUC |
| 1145 | UCUCGGCCCGCUCCCGUCG |
| 1146 | CUCGGCCCGCUCCCGUCGG |
| 1147 | UCGGCCCGCUCCCGUCGGG |
| 1148 | CGGCCCGCUCCCGUCGGGG |
| 1149 | GGCCCGCUCCCGUCGGGGC |
| 1150 | GCCCGCUCCCGUCGGGGCG |
| 1151 | CCCGCUCCCGUCGGGGCGG |
| 1152 | CCGCUCCCGUCGGGGCGGA |
| 1153 | CGCUCCCGUCGGGGCGGAG |
| 1154 | GCUCCCGUCGGGGCGGAGC |
| 1155 | CUCCCGUCGGGGCGGAGCG |
| 1156 | UCCCGUCGGGGCGGAGCGU |
| 1157 | CCCGUCGGGGCGGAGCGUC |
| 1158 | CCGUCGGGGCGGAGCGUCC |
| 1159 | CGUCGGGGCGGAGCGUCCG |
| 1160 | GUCGGGGCGGAGCGUCCGA |
| 1161 | UCGGGGCGGAGCGUCCGAC |
| 1162 | CGGGGCGGAGCGUCCGACG |
| 1163 | GGGGCGGAGCGUCCGACGA |
| 1164 | GGGCGGAGCGUCCGACGAU |
| 1165 | GGCGGAGCGUCCGACGAUC |
| 1166 | GCGGAGCGUCCGACGAUCG |
| 1167 | CGGAGCGUCCGACGAUCGG |
| 1168 | GGAGCGUCCGACGAUCGGC |
| 1169 | GAGCGUCCGACGAUCGGCC |
| 1170 | AGCGUCCGACGAUCGGCCU |
| 1171 | GCGUCCGACGAUCGGCCUC |
| 1172 | CGUCCGACGAUCGGCCUCC |
| 1173 | GUCCGACGAUCGGCCUCCA |
| 1174 | UCCGACGAUCGGCCUCCAC |
| 1175 | CCGACGAUCGGCCUCCACG |
| 1176 | CGACGAUCGGCCUCCACGA |
| 1177 | GACGAUCGGCCUCCACGAA |
| 1178 | ACGAUCGGCCUCCACGAAA |
| 1179 | CGAUCGGCCUCCACGAAAC |
| 1180 | GAUCGGCCUCCACGAAACG |
| 1181 | AUCGGCCUCCACGAAACGC |
| 1182 | UCGGCCUCCACGAAACGCG |
| 1183 | CGGCCUCCACGAAACGCGG |
| 1184 | GGCCUCCACGAAACGCGGU |
| 1185 | GCCUCCACGAAACGCGGUG |
| 1186 | CCUCCACGAAACGCGGUGC |
| 1187 | CUCCACGAAACGCGGUGCC |
| 1188 | UCCACGAAACGCGGUGCCG |
| 1189 | CCACGAAACGCGGUGCCGU |
| 1190 | CACGAAACGCGGUGCCGUG |
| 1191 | ACGAAACGCGGUGCCGUGA |
| 1192 | CGAAACGCGGUGCCGUGAU |
| 1193 | GAAACGCGGUGCCGUGAUG |
| 1194 | AAACGCGGUGCCGUGAUGU |
| 1195 | AACGCGGUGCCGUGAUGUG |
| 1196 | ACGCGGUGCCGUGAUGUGU |
| 1197 | CGCGGUGCCGUGAUGUGUU |
| 1198 | GCGGUGCCGUGAUGUGUUU |
| 1199 | CGGUGCCGUGAUGUGUUUG |
| 1200 | GGUGCCGUGAUGUGUUUGU |
| 1201 | GUGCCGUGAUGUGUUUGUA |
| 1202 | UGCCGUGAUGUGUUUGUAG |
| 1203 | GCCGUGAUGUGUUUGUAGU |
| 1204 | CCGUGAUGUGUUUGUAGUG |
| 1205 | CGUGAUGUGUUUGUAGUGG |
| 1206 | GUGAUGUGUUUGUAGUGGU |
| 1207 | UGAUGUGUUUGUAGUGGUU |
| 1208 | GAUGUGUUUGUAGUGGUUC |
| 1209 | AUGUGUUUGUAGUGGUUCC |
| 1210 | UGUGUUUGUAGUGGUUCCU |
| 1211 | GUGUUUGUAGUGGUUCCUC |
| 1212 | UGUUUGUAGUGGUUCCUCG |
| 1213 | GUUUGUAGUGGUUCCUCGU |
| 1214 | UUUGUAGUGGUUCCUCGUA |
| 1215 | UUGUAGUGGUUCCUCGUAG |
| 1216 | UGUAGUGGUUCCUCGUAGG |
| 1217 | GUAGUGGUUCCUCGUAGGC |
| 1218 | UAGUGGUUCCUCGUAGGCU |
| 1219 | AGUGGUUCCUCGUAGGCUC |
| 1220 | GUGGUUCCUCGUAGGCUCC |
| 1221 | UGGUUCCUCGUAGGCUCCA |
| 1222 | GGUUCCUCGUAGGCUCCAG |
| 1223 | GUUCCUCGUAGGCUCCAGA |
| 1224 | UUCCUCGUAGGCUCCAGAC |
| 1225 | UCCUCGUAGGCUCCAGACG |
| 1226 | CCUCGUAGGCUCCAGACGU |
| 1227 | CUCGUAGGCUCCAGACGUU |
| 1228 | UCGUAGGCUCCAGACGUUU |
| 1229 | CGUAGGCUCCAGACGUUUU |
| 1230 | GUAGGCUCCAGACGUUUUC |
| 1231 | UAGGCUCCAGACGUUUUCU |
| 1232 | AGGCUCCAGACGUUUUCUC |
| 1233 | GGCUCCAGACGUUUUCUCC |
| 1234 | GCUCCAGACGUUUUCUCCU |
| 1235 | CUCCAGACGUUUUCUCCUC |
| 1236 | UCCAGACGUUUUCUCCUCG |
| 1237 | CCAGACGUUUUCUCCUCGU |
| 1238 | CAGACGUUUUCUCCUCGUA |
| 1239 | AGACGUUUUCUCCUCGUAU |
| 1240 | GACGUUUUCUCCUCGUAUC |
| 1241 | ACGUUUUCUCCUCGUAUCG |
| 1242 | CGUUUUCUCCUCGUAUCGC |
| 1243 | GUUUUCUCCUCGUAUCGCC |
| 1244 | UUUUCUCCUCGUAUCGCCA |
| 1245 | UUUCUCCUCGUAUCGCCAA |
| 1246 | UUCUCCUCGUAUCGCCAAA |
| 1247 | UCUCCUCGUAUCGCCAAAU |
| 1248 | CUCCUCGUAUCGCCAAAUU |
| 1249 | UCCUCGUAUCGCCAAAUUA |
| 1250 | CCUCGUAUCGCCAAAUUAA |
| 1251 | CUCGUAUCGCCAAAUUAAC |
| 1252 | UCGUAUCGCCAAAUUAACG |
| 1253 | CGUAUCGCCAAAUUAACGC |
| 1254 | GUAUCGCCAAAUUAACGCG |
| 1255 | UAUCGCCAAAUUAACGCGU |
| 1256 | AUCGCCAAAUUAACGCGUU |
| 1257 | UCGCCAAAUUAACGCGUUU |
| 1258 | CGCCAAAUUAACGCGUUUU |
| 1259 | GCCAAAUUAACGCGUUUUG |
| 1260 | CCAAAUUAACGCGUUUUGC |
| 1261 | CAAAUUAACGCGUUUUGCA |
| 1262 | AAAUUAACGCGUUUUGCAU |
| 1263 | AAUUAACGCGUUUUGCAUA |
| 1264 | AUUAACGCGUUUUGCAUAU |
| 1265 | UUAACGCGUUUUGCAUAUU |
| 1266 | UAACGCGUUUUGCAUAUUA |
| 1267 | AACGCGUUUUGCAUAUUAC |
| 1268 | ACGCGUUUUGCAUAUUACA |
| 1269 | CGCGUUUUGCAUAUUACAG |
| 1270 | GCGUUUUGCAUAUUACAGU |
| 1271 | CGUUUUGCAUAUUACAGUU |
| 1272 | GUUUUGCAUAUUACAGUUG |
| 1273 | UUUUGCAUAUUACAGUUGA |
| 1274 | UUUGCAUAUUACAGUUGAG |
| 1275 | UUGCAUAUUACAGUUGAGU |
| 1276 | UGCAUAUUACAGUUGAGUG |
| 1277 | GCAUAUUACAGUUGAGUGC |
| 1278 | CAUAUUACAGUUGAGUGCC |
| 1279 | AUAUUACAGUUGAGUGCCU |
| 1280 | UAUUACAGUUGAGUGCCUC |
| 1281 | AUUACAGUUGAGUGCCUCG |
| 1282 | UUACAGUUGAGUGCCUCGA |
| 1283 | UACAGUUGAGUGCCUCGAC |
| 1284 | ACAGUUGAGUGCCUCGACU |
| 1285 | CAGUUGAGUGCCUCGACUU |
| 1286 | AGUUGAGUGCCUCGACUUA |
| 1287 | GUUGAGUGCCUCGACUUAG |
| 1288 | UUGAGUGCCUCGACUUAGA |
| 1289 | UGAGUGCCUCGACUUAGAU |
| 1290 | GAGUGCCUCGACUUAGAUU |
| 1291 | AGUGCCUCGACUUAGAUUG |
| 1292 | GUGCCUCGACUUAGAUUGC |
| 1293 | UGCCUCGACUUAGAUUGCA |
| 1294 | GCCUCGACUUAGAUUGCAA |
| 1295 | CCUCGACUUAGAUUGCAAU |
| 1296 | CUCGACUUAGAUUGCAAUA |
| 1297 | UCGACUUAGAUUGCAAUAU |
| 1298 | CGACUUAGAUUGCAAUAUA |
| 1299 | GACUUAGAUUGCAAUAUAA |
| 1300 | ACUUAGAUUGCAAUAUAAG |
| 1301 | CUUAGAUUGCAAUAUAAGC |
| 1302 | UUAGAUUGCAAUAUAAGCG |
| 1303 | UAGAUUGCAAUAUAAGCGG |
| 1304 | AGAUUGCAAUAUAAGCGGC |
| 1305 | GAUUGCAAUAUAAGCGGCC |
| 1306 | AUUGCAAUAUAAGCGGCCA |
| 1307 | UUGCAAUAUAAGCGGCCAG |
| 1308 | UGCAAUAUAAGCGGCCAGC |
| 1309 | GCAAUAUAAGCGGCCAGCA |
| 1310 | CAAUAUAAGCGGCCAGCAA |
| 1311 | AAUAUAAGCGGCCAGCAAA |
| 1312 | AUAUAAGCGGCCAGCAAAC |
| 1313 | UAUAAGCGGCCAGCAAACA |
| 1314 | AUAAGCGGCCAGCAAACAA |
| 1315 | UAAGCGGCCAGCAAACAAG |
| 1316 | AAGCGGCCAGCAAACAAGU |
| 1317 | AGCGGCCAGCAAACAAGUC |
| 1318 | GCGGCCAGCAAACAAGUCU |
| 1319 | CGGCCAGCAAACAAGUCUC |
| 1320 | GGCCAGCAAACAAGUCUCA |
| 1321 | GCCAGCAAACAAGUCUCAA |
| 1322 | CCAGCAAACAAGUCUCAAA |
| 1323 | CAGCAAACAAGUCUCAAAA |
| 1324 | AGCAAACAAGUCUCAAAAA |
| 1325 | GCAAACAAGUCUCAAAAAA |
| 1326 | CAAACAAGUCUCAAAAAAA |
| 1327 | AAACAAGUCUCAAAAAAAA |
| 1328 | AACAAGUCUCAAAAAAAAG |
| 1329 | ACAAGUCUCAAAAAAAAGU |
| 1330 | CAAGUCUCAAAAAAAAGUU |
| 1331 | AAGUCUCAAAAAAAAGUUA |
| 1332 | AGUCUCAAAAAAAAGUUAC |
| 1333 | GUCUCAAAAAAAAGUUACG |
| 1334 | UCUCAAAAAAAAGUUACGU |
| 1335 | CUCAAAAAAAAGUUACGUG |
| 1336 | UCAAAAAAAAGUUACGUGC |
| 1337 | CAAAAAAAAGUUACGUGCG |
| 1338 | AAAAAAAAGUUACGUGCGU |
| 1339 | AAAAAAAGUUACGUGCGUU |
| 1340 | AAAAAAGUUACGUGCGUUU |
| 1341 | AAAAAGUUACGUGCGUUUC |
| 1342 | AAAAGUUACGUGCGUUUCU |
| 1343 | AAAGUUACGUGCGUUUCUG |
| 1344 | AAGUUACGUGCGUUUCUGC |
| 1345 | AGUUACGUGCGUUUCUGCG |
| 1346 | GUUACGUGCGUUUCUGCGA |
| 1347 | UUACGUGCGUUUCUGCGAG |
| 1348 | UACGUGCGUUUCUGCGAGU |
| 1349 | ACGUGCGUUUCUGCGAGUG |
| 1350 | CGUGCGUUUCUGCGAGUGU |
| 1351 | GUGCGUUUCUGCGAGUGUU |
| 1352 | UGCGUUUCUGCGAGUGUUA |
| 1353 | GCGUUUCUGCGAGUGUUAU |
| 1354 | CGUUUCUGCGAGUGUUAUU |
| 1355 | GUUUCUGCGAGUGUUAUUU |
| 1356 | UUUCUGCGAGUGUUAUUUU |
| 1357 | UUCUGCGAGUGUUAUUUUG |
| 1358 | UCUGCGAGUGUUAUUUUGU |
| 1359 | CUGCGAGUGUUAUUUUGUU |
| 1360 | UGCGAGUGUUAUUUUGUUA |
| 1361 | GCGAGUGUUAUUUUGUUAA |
| 1362 | CGAGUGUUAUUUUGUUAAG |
| 1363 | GAGUGUUAUUUUGUUAAGA |
| 1364 | AGUGUUAUUUUGUUAAGAA |
| 1365 | GUGUUAUUUUGUUAAGAAC |
| 1366 | UGUUAUUUUGUUAAGAACG |
| 1367 | GUUAUUUUGUUAAGAACGG |
| 1368 | UUAUUUUGUUAAGAACGGC |
| 1369 | UAUUUUGUUAAGAACGGCU |
| 1370 | AUUUUGUUAAGAACGGCUC |
| 1371 | UUUUGUUAAGAACGGCUCA |
| 1372 | UUUGUUAAGAACGGCUCAC |
| 1373 | UUGUUAAGAACGGCUCACA |
| 1374 | UGUUAAGAACGGCUCACAG |
| 1375 | GUUAAGAACGGCUCACAGU |
| 1376 | UUAAGAACGGCUCACAGUG |
| 1377 | UAAGAACGGCUCACAGUGU |
| 1378 | AAGAACGGCUCACAGUGUC |
| 1379 | AGAACGGCUCACAGUGUCC |
| 1380 | GAACGGCUCACAGUGUCCU |
| 1381 | AACGGCUCACAGUGUCCUC |
| 1382 | ACGGCUCACAGUGUCCUCU |
| 1383 | CGGCUCACAGUGUCCUCUU |
| 1384 | GGCUCACAGUGUCCUCUUC |
| 1385 | GCUCACAGUGUCCUCUUCC |
| 1386 | CUCACAGUGUCCUCUUCCU |
| 1387 | UCACAGUGUCCUCUUCCUG |
| 1388 | CACAGUGUCCUCUUCCUGU |
| 1389 | ACAGUGUCCUCUUCCUGUG |
| 1390 | CAGUGUCCUCUUCCUGUGU |
| 1391 | AGUGUCCUCUUCCUGUGUU |
| 1392 | GUGUCCUCUUCCUGUGUUA |
| 1393 | UGUCCUCUUCCUGUGUUAC |
| 1394 | GUCCUCUUCCUGUGUUACA |
| 1395 | UCCUCUUCCUGUGUUACAG |
| 1396 | CCUCUUCCUGUGUUACAGA |
| 1397 | CUCUUCCUGUGUUACAGAA |
| 1398 | UCUUCCUGUGUUACAGAAG |
| 1399 | CUUCCUGUGUUACAGAAGC |
| 1400 | UUCCUGUGUUACAGAAGCC |
| 1401 | UCCUGUGUUACAGAAGCCA |
| 1402 | CCUGUGUUACAGAAGCCAA |
| 1403 | CUGUGUUACAGAAGCCAAC |
| 1404 | UGUGUUACAGAAGCCAACC |
| 1405 | GUGUUACAGAAGCCAACCU |
| 1406 | UGUUACAGAAGCCAACCUG |
| 1407 | GUUACAGAAGCCAACCUGA |
| 1408 | UUACAGAAGCCAACCUGAA |
| 1409 | UACAGAAGCCAACCUGAAA |
| 1410 | ACAGAAGCCAACCUGAAAU |
| 1411 | CAGAAGCCAACCUGAAAUG |
| 1412 | AGAAGCCAACCUGAAAUGA |
| 1413 | GAAGCCAACCUGAAAUGAA |
| 1414 | AAGCCAACCUGAAAUGAAA |
| 1415 | AGCCAACCUGAAAUGAAAC |
| 1416 | GCCAACCUGAAAUGAAACU |
| 1417 | CCAACCUGAAAUGAAACUA |
| 1418 | CAACCUGAAAUGAAACUAG |
| 1419 | AACCUGAAAUGAAACUAGU |
| 1420 | ACCUGAAAUGAAACUAGUC |
| 1421 | CCUGAAAUGAAACUAGUCU |
| 1422 | CUGAAAUGAAACUAGUCUG |
| 1423 | UGAAAUGAAACUAGUCUGG |
| 1424 | GAAAUGAAACUAGUCUGGA |
| 1425 | AAAUGAAACUAGUCUGGAA |
| 1426 | AAUGAAACUAGUCUGGAAA |
| 1427 | AUGAAACUAGUCUGGAAAA |
| 1428 | UGAAACUAGUCUGGAAAAA |
| 1429 | GAAACUAGUCUGGAAAAAU |
| 1430 | AAACUAGUCUGGAAAAAUU |
| 1431 | AACUAGUCUGGAAAAAUUC |
| 1432 | ACUAGUCUGGAAAAAUUCA |
| 1433 | CUAGUCUGGAAAAAUUCAU |
| 1434 | UAGUCUGGAAAAAUUCAUU |
| 1435 | AGUCUGGAAAAAUUCAUUG |
| 1436 | GUCUGGAAAAAUUCAUUGU |
| 1437 | UCUGGAAAAAUUCAUUGUU |
| 1438 | CUGGAAAAAUUCAUUGUUC |
| 1439 | UGGAAAAAUUCAUUGUUCU |
| 1440 | GGAAAAAUUCAUUGUUCUC |
| 1441 | GAAAAAUUCAUUGUUCUCU |
| 1442 | AAAAAUUCAUUGUUCUCUG |
| 1443 | AAAAUUCAUUGUUCUCUGU |
| 1444 | AAAUUCAUUGUUCUCUGUA |
| 1445 | AAUUCAUUGUUCUCUGUAG |
| 1446 | AUUCAUUGUUCUCUGUAGU |
| 1447 | UUCAUUGUUCUCUGUAGUU |
| 1448 | UCAUUGUUCUCUGUAGUUG |
| 1449 | CAUUGUUCUCUGUAGUUGC |
| 1450 | AUUGUUCUCUGUAGUUGCA |
| 1451 | UUGUUCUCUGUAGUUGCAG |
| 1452 | UGUUCUCUGUAGUUGCAGC |
| 1453 | GUUCUCUGUAGUUGCAGCU |
| 1454 | UUCUCUGUAGUUGCAGCUG |
| 1455 | UCUCUGUAGUUGCAGCUGU |
| 1456 | CUCUGUAGUUGCAGCUGUA |
| 1457 | UCUGUAGUUGCAGCUGUAC |
| 1458 | CUGUAGUUGCAGCUGUACC |
| 1459 | UGUAGUUGCAGCUGUACCU |
| 1460 | GUAGUUGCAGCUGUACCUG |
| 1461 | UAGUUGCAGCUGUACCUGA |
| 1462 | AGUUGCAGCUGUACCUGAA |
| 1463 | GUUGCAGCUGUACCUGAAA |
| 1464 | UUGCAGCUGUACCUGAAAU |
| 1465 | UGCAGCUGUACCUGAAAUA |
| 1466 | GCAGCUGUACCUGAAAUAA |
| 1467 | CAGCUGUACCUGAAAUAAA |
| 1468 | AGCUGUACCUGAAAUAAAA |
| 1469 | GCUGUACCUGAAAUAAAAA |
| 1470 | CUGUACCUGAAAUAAAAAU |
| 1471 | UGUACCUGAAAUAAAAAUG |
| 1472 | GUACCUGAAAUAAAAAUGU |
| 1473 | UACCUGAAAUAAAAAUGUU |
| 1474 | ACCUGAAAUAAAAAUGUUA |
| 1475 | CCUGAAAUAAAAAUGUUAU |
| 1476 | CUGAAAUAAAAAUGUUAUU |
| 1477 | UGAAAUAAAAAUGUUAUUG |
| 1478 | GAAAUAAAAAUGUUAUUGA |
| 1479 | AAAUAAAAAUGUUAUUGAU |
| 1480 | AAUAAAAAUGUUAUUGAUG |
| 1481 | AUAAAAAUGUUAUUGAUGA |
| 1482 | UAAAAAUGUUAUUGAUGAC |
| 1483 | AAAAAUGUUAUUGAUGACU |
| 1484 | AAAAUGUUAUUGAUGACUG |
| 1485 | AAAUGUUAUUGAUGACUGA |
| 1486 | AAUGUUAUUGAUGACUGAA |
| 1487 | AUGUUAUUGAUGACUGAAA |
| 1488 | UGUUAUUGAUGACUGAAAA |
| 1489 | GUUAUUGAUGACUGAAAAA |
| 1490 | UUAUUGAUGACUGAAAAAA |
| 1491 | UAUUGAUGACUGAAAAAAA |
| 1492 | AUUGAUGACUGAAAAAAAA |
| 1493 | UUGAUGACUGAAAAAAAA |
| 1494 | UGAUGACUGAAAAAAAAA |
| 1495 | GAUGACUGAAAAAAAAAA |
| 1496 | AUGACUGAAAAAAAAAAA |
| 1497 | UGACUGAAAAAAAAAAAA |
| 1498 | GACUGAAAAAAAAAAAAA |
| 1499 | ACUGAAAAAAAAAAAAAA |
| 1500 | CUGAAAAAAAAAAAAAAA |
| 1501 | UGAAAAAAAAAAAAAAAA |
| 1502 | AAAACAGUGGCAGAUGAAA |
| 1503 | AAACAGUGGCAGAUGAAAG |
| 1504 | AACAGUGGCAGAUGAAAGG |
| 1505 | ACAGUGGCAGAUGAAAGGG |
| 1506 | CAGUGGCAGAUGAAAGGGA |
| 1507 | AGUGGCAGAUGAAAGGGAU |
| 1508 | GUGGCAGAUGAAAGGGAUG |
| 1509 | UGGCAGAUGAAAGGGAUGG |
| 1510 | GGCAGAUGAAAGGGAUGGA |
| 1511 | GCAGAUGAAAGGGAUGGAG |
| 1512 | CAGAUGAAAGGGAUGGAGA |
| 1513 | AGAUGAAAGGGAUGGAGAA |
| 1514 | GAUGAAAGGGAUGGAGAAC |
| 1515 | AUGAAAGGGAUGGAGAACC |
| 1516 | UGAAAGGGAUGGAGAACCG |
| 1517 | GAAAGGGAUGGAGAACCGC |
| 1518 | AAAGGGAUGGAGAACCGCC |
| 1519 | AAGGGAUGGAGAACCGCCA |
| 1520 | AGGGAUGGAGAACCGCCAU |
| 1521 | GGGAUGGAGAACCGCCAUG |
| 1522 | GGAUGGAGAACCGCCAUGC |
| 1523 | GAUGGAGAACCGCCAUGCC |
| 1524 | AUGCCCGGUGACUCCCCGG |
| 1525 | UGCCCGGUGACUCCCCGGC |
| 1526 | GCCCGGUGACUCCCCGGCC |
| 1527 | CCCGGUGACUCCCCGGCCG |
| 1528 | CCGGUGACUCCCCGGCCGU |
| 1529 | CGGUGACUCCCCGGCCGUC |
| 1530 | GGUGACUCCCCGGCCGUCA |
| 1531 | GUGACUCCCCGGCCGUCAG |
| 1532 | UGACUCCCCGGCCGUCAGC |
| 1533 | GACUCCCCGGCCGUCAGCA |
| 1534 | ACUCCCCGGCCGUCAGCAA |
| 1535 | CUCCCCGGCCGUCAGCAAG |
| 1536 | UCCCCGGCCGUCAGCAAGC |
| 1537 | CCCCGGCCGUCAGCAAGCC |
| 1538 | CCCGGCCGUCAGCAAGCCC |
| 1539 | CCGGCCGUCAGCAAGCCCC |
| 1540 | CGGCCGUCAGCAAGCCCCC |
| 1541 | GGCCGUCAGCAAGCCCCCG |
| 1542 | GCCGUCAGCAAGCCCCCGG |
| 1543 | CCGUCAGCAAGCCCCCGGA |
| 1544 | CGUCAGCAAGCCCCCGGAC |
| 1545 | GUCAGCAAGCCCCCGGACG |
| 1546 | UCAGCAAGCCCCCGGACGG |
| 1547 | CAGCAAGCCCCCGGACGGC |
| 1548 | AGCAAGCCCCCGGACGGCG |
| 1549 | GCAAGCCCCCGGACGGCGC |
| 1550 | CAAGCCCCCGGACGGCGCC |
| 1551 | AAGCCCCCGGACGGCGCCG |
| 1552 | AGCCCCCGGACGGCGCCGG |
| 1553 | GCCCCCGGACGGCGCCGGG |
| 1554 | CCCCCGGACGGCGCCGGGC |
| 1555 | CCCCGGACGGCGCCGGGCC |
| 1556 | CCCGGACGGCGCCGGGCCG |
| 1557 | CCGGACGGCGCCGGGCCGG |
| 1558 | CGGACGGCGCCGGGCCGGG |
| 1559 | GGACGGCGCCGGGCCGGGG |
| 1560 | GACGGCGCCGGGCCGGGGG |
| 1561 | ACGGCGCCGGGCCGGGGGA |
| 1562 | CGGCGCCGGGCCGGGGGAC |
| 1563 | GGCGCCGGGCCGGGGGACA |
| 1564 | GCGCCGGGCCGGGGGACAA |
| 1565 | CGCCGGGCCGGGGGACAAG |
| 1566 | GCCGGGCCGGGGGACAAGG |
| 1567 | CCGGGCCGGGGGACAAGGC |
| 1568 | CGGGCCGGGGGACAAGGCG |
| 1569 | GGGCCGGGGGACAAGGCGG |
| 1570 | GGCCGGGGGACAAGGCGGG |
| 1571 | GCCGGGGGACAAGGCGGGC |
| 1572 | CCGGGGGACAAGGCGGGCG |
| 1573 | CGGGGGACAAGGCGGGCGG |
| 1574 | GGGGGACAAGGCGGGCGGC |
| 1575 | GGGGACAAGGCGGGCGGCC |
| 1576 | GGGACAAGGCGGGCGGCCU |
| 1577 | GGACAAGGCGGGCGGCCUC |
| 1578 | GACAAGGCGGGCGGCCUCG |
| 1579 | ACAAGGCGGGCGGCCUCGG |
| 1580 | CAAGGCGGGCGGCCUCGGC |
| 1581 | AAGGCGGGCGGCCUCGGCA |
| 1582 | AGGCGGGCGGCCUCGGCAA |
| 1583 | GGCGGGCGGCCUCGGCAAG |
| 1584 | GCGGGCGGCCUCGGCAAGG |
| 1585 | CGGGCGGCCUCGGCAAGGC |
| 1586 | GGGCGGCCUCGGCAAGGCG |
| 1587 | GGCGGCCUCGGCAAGGCGG |
| 1588 | GCGGCCUCGGCAAGGCGGC |
| 1589 | CGGCCUCGGCAAGGCGGCG |
| 1590 | GGCCUCGGCAAGGCGGCGG |
| 1591 | GCCUCGGCAAGGCGGCGGC |
| 1592 | CCUCGGCAAGGCGGCGGCC |
| 1593 | CUCGGCAAGGCGGCGGCCC |
| 1594 | UCGGCAAGGCGGCGGCCCA |
| 1595 | CGGCAAGGCGGCGGCCCAA |
| 1596 | GGCAAGGCGGCGGCCCAAA |
| 1597 | GCAAGGCGGCGGCCCAAAU |
| 1598 | CAAGGCGGCGGCCCAAAUG |
| 1599 | AAGGCGGCGGCCCAAAUGG |
| 1600 | AGGCGGCGGCCCAAAUGGC |
| 1601 | GGCGGCGGCCCAAAUGGCG |
| 1602 | GCGGCGGCCCAAAUGGCGG |
| 1603 | CGGCGGCCCAAAUGGCGGC |
| 1604 | GGCGGCCCAAAUGGCGGCC |
| 1605 | GCGGCCCAAAUGGCGGCCG |
| 1606 | CGGCCCAAAUGGCGGCCGC |
| 1607 | GGCCCAAAUGGCGGCCGCC |
| 1608 | GCCCAAAUGGCGGCCGCCC |
| 1609 | CCCAAAUGGCGGCCGCCCC |
| 1610 | CCAAAUGGCGGCCGCCCCG |
| 1611 | CAAAUGGCGGCCGCCCCGG |
| 1612 | AAAUGGCGGCCGCCCCGGC |
| 1613 | AAUGGCGGCCGCCCCGGCG |
| 1614 | AUGGCGGCCGCCCCGGCGG |
| 1615 | UGGCGGCCGCCCCGGCGGC |
| 1616 | GGCGGCCGCCCCGGCGGCG |
| 1617 | GCGGCCGCCCCGGCGGCGG |
| 1618 | CGGCCGCCCCGGCGGCGGG |
| 1619 | GGCCGCCCCGGCGGCGGGC |
| 1620 | GCCGCCCCGGCGGCGGGCA |
| 1621 | CCGCCCCGGCGGCGGGCAA |
| 1622 | CGCCCCGGCGGCGGGCAAG |
| 1623 | GCCCCGGCGGCGGGCAAGA |
| 1624 | CCCCGGCGGCGGGCAAGAA |
| 1625 | CCCGGCGGCGGGCAAGAAG |
| 1626 | GGCAGCGGGUGAUGGCCGC |
| 1627 | GCAGCGGGUGAUGGCCGCG |
| 1628 | CAGCGGGUGAUGGCCGCGC |
| 1629 | AGCGGGUGAUGGCCGCGCA |
| 1630 | GCGGGUGAUGGCCGCGCAG |
| 1631 | CGGGUGAUGGCCGCGCAGG |
| 1632 | GGGUGAUGGCCGCGCAGGU |
| 1633 | GGUGAUGGCCGCGCAGGUU |
| 1634 | GUGAUGGCCGCGCAGGUUG |
| 1635 | UGAUGGCCGCGCAGGUUGC |
| 1636 | GAUGGCCGCGCAGGUUGCA |
| 1637 | AUGGCCGCGCAGGUUGCAC |
| 1638 | UGGCCGCGCAGGUUGCACU |
| 1639 | GGCCGCGCAGGUUGCACUG |
| 1640 | GCCGCGCAGGUUGCACUGA |
| 1641 | CCGCGCAGGUUGCACUGAG |
| 1642 | CGCGCAGGUUGCACUGAGG |
| 1643 | GCGCAGGUUGCACUGAGGA |
| 1644 | CGCAGGUUGCACUGAGGAG |

TABLE 2
DsRNA molecules targeting mRNA encoding chicken
ASW (WPKCI).

| SEQ ID NO | Sequence 5′-3′ |
|---|---|
| 1645 | CGUCUGCGGGUGCCUUGCG |
| 1646 | GUCUGCGGGUGCCUUGCGA |
| 1647 | UCUGCGGGUGCCUUGCGAU |
| 1648 | CUGCGGGUGCCUUGCGAUA |
| 1649 | UGCGGGUGCCUUGCGAUAC |
| 1650 | GCGGGUGCCUUGCGAUACG |
| 1651 | CGGGUGCCUUGCGAUACGU |
| 1652 | GGGUGCCUUGCGAUACGUC |
| 1653 | GGUGCCUUGCGAUACGUCG |
| 1654 | GUGCCUUGCGAUACGUCGG |
| 1655 | UGCCUUGCGAUACGUCGGC |
| 1656 | GCCUUGCGAUACGUCGGCA |
| 1657 | CCUUGCGAUACGUCGGCAU |
| 1658 | CUUGCGAUACGUCGGCAUG |
| 1659 | UUGCGAUACGUCGGCAUGG |
| 1660 | UGCGAUACGUCGGCAUGGC |
| 1661 | GCGAUACGUCGGCAUGGCU |
| 1662 | CGAUACGUCGGCAUGGCUG |
| 1663 | GAUACGUCGGCAUGGCUGA |
| 1664 | AUACGUCGGCAUGGCUGAC |
| 1665 | UACGUCGGCAUGGCUGACG |
| 1666 | ACGUCGGCAUGGCUGACGA |
| 1667 | CGUCGGCAUGGCUGACGAG |
| 1668 | GUCGGCAUGGCUGACGAGA |
| 1669 | UCGGCAUGGCUGACGAGAU |
| 1670 | CGGCAUGGCUGACGAGAUC |
| 1671 | GGCAUGGCUGACGAGAUCC |
| 1672 | GCAUGGCUGACGAGAUCCG |
| 1673 | CAUGGCUGACGAGAUCCGC |
| 1674 | AUGGCUGACGAGAUCCGCA |
| 1675 | UGGCUGACGAGAUCCGCAA |
| 1676 | GGCUGACGAGAUCCGCAAG |
| 1677 | GCUGACGAGAUCCGCAAGG |
| 1678 | CUGACGAGAUCCGCAAGGC |
| 1679 | UGACGAGAUCCGCAAGGCG |
| 1680 | GACGAGAUCCGCAAGGCGC |
| 1681 | ACGAGAUCCGCAAGGCGCA |
| 1682 | CGAGAUCCGCAAGGCGCAG |
| 1683 | GAGAUCCGCAAGGCGCAGG |
| 1684 | AGAUCCGCAAGGCGCAGGC |
| 1685 | GAUCCGCAAGGCGCAGGCC |
| 1686 | AUCCGCAAGGCGCAGGCCG |
| 1687 | UCCGCAAGGCGCAGGCCGC |
| 1688 | CCGCAAGGCGCAGGCCGCG |
| 1689 | CGCAAGGCGCAGGCCGCGC |
| 1690 | GCAAGGCGCAGGCCGCGCG |
| 1691 | CAAGGCGCAGGCCGCGCGC |
| 1692 | AAGGCGCAGGCCGCGCGCC |
| 1693 | AGGCGCAGGCCGCGCGCCC |
| 1694 | GGCGCAGGCCGCGCGCCCU |
| 1695 | GCGCAGGCCGCGCGCCCUG |
| 1696 | CGCAGGCCGCGCGCCCUGG |
| 1697 | GCAGGCCGCGCGCCCUGGU |
| 1698 | CAGGCCGCGCGCCCUGGUG |
| 1699 | AGGCCGCGCGCCCUGGUGG |
| 1700 | GGCCGCGCGCCCUGGUGGG |
| 1701 | GCCGCGCGCCCUGGUGGGG |
| 1702 | CCGCGCGCCCUGGUGGGGA |
| 1703 | CGCGCGCCCUGGUGGGGAC |
| 1704 | GCGCGCCCUGGUGGGGACA |
| 1705 | CGCGCCCUGGUGGGGACAC |
| 1706 | GCGCCCUGGUGGGGACACC |
| 1707 | CGCCCUGGUGGGGACACCA |
| 1708 | GCCCUGGUGGGGACACCAU |
| 1709 | CCCUGGUGGGGACACCAUC |
| 1710 | CCUGGUGGGGACACCAUCU |
| 1711 | CUGGUGGGGACACCAUCUU |
| 1712 | UGGUGGGGACACCAUCUUC |
| 1713 | GGUGGGGACACCAUCUUCG |
| 1714 | GUGGGGACACCAUCUUCGG |
| 1715 | UGGGGACACCAUCUUCGGG |
| 1716 | GGGGACACCAUCUUCGGGA |
| 1717 | GGGACACCAUCUUCGGGAA |
| 1718 | GGACACCAUCUUCGGGAAG |
| 1719 | GACACCAUCUUCGGGAAGA |
| 1720 | ACACCAUCUUCGGGAAGAU |
| 1721 | CACCAUCUUCGGGAAGAUU |
| 1722 | ACCAUCUUCGGGAAGAUUA |
| 1723 | CCAUCUUCGGGAAGAUUAU |
| 1724 | CAUCUUCGGGAAGAUUAUC |
| 1725 | AUCUUCGGGAAGAUUAUCC |
| 1726 | UCUUCGGGAAGAUUAUCCG |
| 1727 | CUUCGGGAAGAUUAUCCGC |
| 1728 | UUCGGGAAGAUUAUCCGCA |
| 1729 | UCGGGAAGAUUAUCCGCAA |
| 1730 | CGGGAAGAUUAUCCGCAAG |
| 1731 | GGGAAGAUUAUCCGCAAGG |
| 1732 | GGAAGAUUAUCCGCAAGGA |
| 1733 | GAAGAUUAUCCGCAAGGAG |
| 1734 | AAGAUUAUCCGCAAGGAGA |
| 1735 | AGAUUAUCCGCAAGGAGAU |
| 1736 | GAUUAUCCGCAAGGAGAUU |
| 1737 | AUUAUCCGCAAGGAGAUUC |
| 1738 | UUAUCCGCAAGGAGAUUCC |
| 1739 | UAUCCGCAAGGAGAUUCCC |
| 1740 | AUCCGCAAGGAGAUUCCCG |
| 1741 | UCCGCAAGGAGAUUCCCGC |
| 1742 | CCGCAAGGAGAUUCCCGCC |
| 1743 | CGCAAGGAGAUUCCCGCCA |
| 1744 | GCAAGGAGAUUCCCGCCAA |
| 1745 | CAAGGAGAUUCCCGCCAAC |
| 1746 | AAGGAGAUUCCCGCCAACA |
| 1747 | AGGAGAUUCCCGCCAACAU |
| 1748 | GGAGAUUCCCGCCAACAUA |
| 1749 | GAGAUUCCCGCCAACAUAA |
| 1750 | AGAUUCCCGCCAACAUAAU |
| 1751 | GAUUCCCGCCAACAUAAUC |
| 1752 | AUUCCCGCCAACAUAAUCU |
| 1753 | UUCCCGCCAACAUAAUCUA |
| 1754 | UCCCGCCAACAUAAUCUAC |
| 1755 | CCCGCCAACAUAAUCUACG |
| 1756 | CCGCCAACAUAAUCUACGA |
| 1757 | CGCCAACAUAAUCUACGAG |
| 1758 | GCCAACAUAAUCUACGAGG |
| 1759 | CCAACAUAAUCUACGAGGA |
| 1760 | CAACAUAAUCUACGAGGAC |
| 1761 | AACAUAAUCUACGAGGACG |
| 1762 | ACAUAAUCUACGAGGACGA |
| 1763 | CAUAAUCUACGAGGACGAG |
| 1764 | AUAAUCUACGAGGACGAGC |
| 1765 | UAAUCUACGAGGACGAGCA |
| 1766 | AAUCUACGAGGACGAGCAG |
| 1767 | AUCUACGAGGACGAGCAGU |
| 1768 | UCUACGAGGACGAGCAGUG |
| 1769 | CUACGAGGACGAGCAGUGC |
| 1770 | UACGAGGACGAGCAGUGCC |
| 1771 | ACGAGGACGAGCAGUGCCU |
| 1772 | CGAGGACGAGCAGUGCCUU |
| 1773 | GAGGACGAGCAGUGCCUUG |
| 1774 | AGGACGAGCAGUGCCUUGC |
| 1775 | GGACGAGCAGUGCCUUGCG |
| 1776 | GACGAGCAGUGCCUUGCGU |
| 1777 | ACGAGCAGUGCCUUGCGUU |
| 1778 | CGAGCAGUGCCUUGCGUUC |
| 1779 | GAGCAGUGCCUUGCGUUCC |
| 1780 | AGCAGUGCCUUGCGUUCCA |
| 1781 | GCAGUGCCUUGCGUUCCAU |
| 1782 | CAGUGCCUUGCGUUCCAUG |
| 1783 | AGUGCCUUGCGUUCCAUGA |
| 1784 | GUGCCUUGCGUUCCAUGAU |
| 1785 | UGCCUUGCGUUCCAUGAUA |
| 1786 | GCCUUGCGUUCCAUGAUAU |
| 1787 | CCUUGCGUUCCAUGAUAUC |
| 1788 | CUUGCGUUCCAUGAUAUCU |
| 1789 | UUGCGUUCCAUGAUAUCUC |
| 1790 | UGCGUUCCAUGAUAUCUCA |
| 1791 | GCGUUCCAUGAUAUCUCAC |
| 1792 | CGUUCCAUGAUAUCUCACC |
| 1793 | GUUCCAUGAUAUCUCACCC |
| 1794 | UUCCAUGAUAUCUCACCCC |
| 1795 | UCCAUGAUAUCUCACCCCA |
| 1796 | CCAUGAUAUCUCACCCCAA |
| 1797 | CAUGAUAUCUCACCCCAAG |
| 1798 | AUGAUAUCUCACCCCAAGC |
| 1799 | UGAUAUCUCACCCCAAGCU |
| 1800 | GAUAUCUCACCCCAAGCUC |
| 1801 | AUAUCUCACCCCAAGCUCC |
| 1802 | UAUCUCACCCCAAGCUCCA |
| 1803 | AUCUCACCCCAAGCUCCAA |
| 1804 | UCUCACCCCAAGCUCCAAC |
| 1805 | CUCACCCCAAGCUCCAACG |
| 1806 | UCACCCCAAGCUCCAACGC |
| 1807 | CACCCCAAGCUCCAACGCA |
| 1808 | ACCCCAAGCUCCAACGCAU |
| 1809 | CCCCAAGCUCCAACGCAUU |
| 1810 | CCCAAGCUCCAACGCAUUU |
| 1811 | CCAAGCUCCAACGCAUUUU |
| 1812 | CAAGCUCCAACGCAUUUUC |
| 1813 | AAGCUCCAACGCAUUUUCU |
| 1814 | AGCUCCAACGCAUUUUCUA |
| 1815 | GCUCCAACGCAUUUUCUAG |
| 1816 | CUCCAACGCAUUUUCUAGU |
| 1817 | UCCAACGCAUUUUCUAGUG |
| 1818 | CCAACGCAUUUUCUAGUGA |
| 1819 | CAACGCAUUUUCUAGUGAU |
| 1820 | AACGCAUUUUCUAGUGAUU |
| 1821 | ACGCAUUUUCUAGUGAUUC |
| 1822 | CGCAUUUUCUAGUGAUUCC |
| 1823 | GCAUUUUCUAGUGAUUCCU |
| 1824 | CAUUUUCUAGUGAUUCCUA |
| 1825 | AUUUUCUAGUGAUUCCUAA |
| 1826 | UUUUCUAGUGAUUCCUAAG |
| 1827 | UUUCUAGUGAUUCCUAAGA |
| 1828 | UUCUAGUGAUUCCUAAGAA |
| 1829 | UCUAGUGAUUCCUAAGAAG |
| 1830 | CUAGUGAUUCCUAAGAAGC |
| 1831 | UAGUGAUUCCUAAGAAGCC |
| 1832 | AGUGAUUCCUAAGAAGCCA |
| 1833 | GUGAUUCCUAAGAAGCCAA |
| 1834 | UGAUUCCUAAGAAGCCAAU |
| 1835 | GAUUCCUAAGAAGCCAAUU |
| 1836 | AUUCCUAAGAAGCCAAUUG |
| 1837 | UUCCUAAGAAGCCAAUUGU |
| 1838 | UCCUAAGAAGCCAAUUGUC |
| 1839 | CCUAAGAAGCCAAUUGUCA |
| 1840 | CUAAGAAGCCAAUUGUCAG |
| 1841 | UAAGAAGCCAAUUGUCAGG |
| 1842 | AAGAAGCCAAUUGUCAGGU |
| 1843 | AGAAGCCAAUUGUCAGGUU |
| 1844 | GAAGCCAAUUGUCAGGUUA |
| 1845 | AAGCCAAUUGUCAGGUUAU |
| 1846 | AGCCAAUUGUCAGGUUAUC |
| 1847 | GCCAAUUGUCAGGUUAUCU |
| 1848 | CCAAUUGUCAGGUUAUCUG |
| 1849 | CAAUUGUCAGGUUAUCUGA |
| 1850 | AAUUGUCAGGUUAUCUGAA |
| 1851 | AUUGUCAGGUUAUCUGAAG |
| 1852 | UUGUCAGGUUAUCUGAAGC |
| 1853 | UGUCAGGUUAUCUGAAGCA |
| 1854 | GUCAGGUUAUCUGAAGCAG |
| 1855 | UCAGGUUAUCUGAAGCAGA |
| 1856 | CAGGUUAUCUGAAGCAGAA |
| 1857 | AGGUUAUCUGAAGCAGAAG |
| 1858 | GGUUAUCUGAAGCAGAAGA |
| 1859 | GUUAUCUGAAGCAGAAGAU |
| 1860 | UUAUCUGAAGCAGAAGAUU |
| 1861 | UAUCUGAAGCAGAAGAUUC |
| 1862 | AUCUGAAGCAGAAGAUUCU |
| 1863 | UCUGAAGCAGAAGAUUCUG |
| 1864 | CUGAAGCAGAAGAUUCUGA |
| 1865 | UGAAGCAGAAGAUUCUGAU |
| 1866 | GAAGCAGAAGAUUCUGAUG |
| 1867 | AAGCAGAAGAUUCUGAUGA |
| 1868 | AGCAGAAGAUUCUGAUGAA |
| 1869 | GCAGAAGAUUCUGAUGAAU |
| 1870 | CAGAAGAUUCUGAUGAAUC |
| 1871 | AGAAGAUUCUGAUGAAUCU |
| 1872 | GAAGAUUCUGAUGAAUCUC |
| 1873 | AAGAUUCUGAUGAAUCUCU |
| 1874 | AGAUUCUGAUGAAUCUCUU |
| 1875 | GAUUCUGAUGAAUCUCUUC |
| 1876 | AUUCUGAUGAAUCUCUUCU |
| 1877 | UUCUGAUGAAUCUCUUCUG |
| 1878 | UCUGAUGAAUCUCUUCUGG |
| 1879 | CUGAUGAAUCUCUUCUGGG |
| 1880 | UGAUGAAUCUCUUCUGGGG |
| 1881 | GAUGAAUCUCUUCUGGGGC |
| 1882 | AUGAAUCUCUUCUGGGGCA |
| 1883 | UGAAUCUCUUCUGGGGCAU |
| 1884 | GAAUCUCUUCUGGGGCAUU |
| 1885 | AAUCUCUUCUGGGGCAUUU |
| 1886 | AUCUCUUCUGGGGCAUUUA |
| 1887 | UCUCUUCUGGGGCAUUUAA |
| 1888 | CUCUUCUGGGGCAUUUAAU |
| 1889 | UCUUCUGGGGCAUUUAAUG |
| 1890 | CUUCUGGGGCAUUUAAUGA |
| 1891 | UUCUGGGGCAUUUAAUGAU |
| 1892 | UCUGGGGCAUUUAAUGAUU |
| 1893 | CUGGGGCAUUUAAUGAUUG |
| 1894 | UGGGGCAUUUAAUGAUUGU |
| 1895 | GGGGCAUUUAAUGAUUGUU |
| 1896 | GGGCAUUUAAUGAUUGUUG |
| 1897 | GGCAUUUAAUGAUUGUUGG |
| 1898 | GCAUUUAAUGAUUGUUGGC |
| 1899 | CAUUUAAUGAUUGUUGGCA |
| 1900 | AUUUAAUGAUUGUUGGCAA |
| 1901 | UUUAAUGAUUGUUGGCAAG |
| 1902 | UUAAUGAUUGUUGGCAAGA |
| 1903 | UAAUGAUUGUUGGCAAGAA |
| 1904 | AAUGAUUGUUGGCAAGAAG |
| 1905 | AUGAUUGUUGGCAAGAAGU |
| 1906 | UGAUUGUUGGCAAGAAGUG |
| 1907 | GAUUGUUGGCAAGAAGUGU |
| 1908 | AUUGUUGGCAAGAAGUGUG |
| 1909 | UUGUUGGCAAGAAGUGUGC |
| 1910 | UGUUGGCAAGAAGUGUGCU |
| 1911 | GUUGGCAAGAAGUGUGCUG |
| 1912 | UUGGCAAGAAGUGUGCUGC |
| 1913 | UGGCAAGAAGUGUGCUGCU |
| 1914 | GGCAAGAAGUGUGCUGCUA |
| 1915 | GCAAGAAGUGUGCUGCUAA |
| 1916 | CAAGAAGUGUGCUGCUAAC |
| 1917 | AAGAAGUGUGCUGCUAACC |
| 1918 | AGAAGUGUGCUGCUAACCU |
| 1919 | GAAGUGUGCUGCUAACCUG |
| 1920 | AAGUGUGCUGCUAACCUGG |
| 1921 | AGUGUGCUGCUAACCUGGG |
| 1922 | GUGUGCUGCUAACCUGGGC |
| 1923 | UGUGCUGCUAACCUGGGCC |
| 1924 | GUGCUGCUAACCUGGGCCU |
| 1925 | UGCUGCUAACCUGGGCCUG |
| 1926 | GCUGCUAACCUGGGCCUGA |
| 1927 | CUGCUAACCUGGGCCUGAC |
| 1928 | UGCUAACCUGGGCCUGACC |
| 1929 | GCUAACCUGGGCCUGACCA |
| 1930 | CUAACCUGGGCCUGACCAA |
| 1931 | UAACCUGGGCCUGACCAAU |
| 1932 | AACCUGGGCCUGACCAAUG |
| 1933 | ACCUGGGCCUGACCAAUGG |
| 1934 | CCUGGGCCUGACCAAUGGA |
| 1935 | CUGGGCCUGACCAAUGGAU |
| 1936 | UGGGCCUGACCAAUGGAUU |
| 1937 | GGGCCUGACCAAUGGAUUC |
| 1938 | GGCCUGACCAAUGGAUUCC |
| 1939 | GCCUGACCAAUGGAUUCCG |
| 1940 | CCUGACCAAUGGAUUCCGG |
| 1941 | CUGACCAAUGGAUUCCGGA |
| 1942 | UGACCAAUGGAUUCCGGAU |
| 1943 | GACCAAUGGAUUCCGGAUG |
| 1944 | ACCAAUGGAUUCCGGAUGG |
| 1945 | CCAAUGGAUUCCGGAUGGU |
| 1946 | CAAUGGAUUCCGGAUGGUU |
| 1947 | AAUGGAUUCCGGAUGGUUU |
| 1948 | AUGGAUUCCGGAUGGUUUU |
| 1949 | UGGAUUCCGGAUGGUUUUG |
| 1950 | GGAUUCCGGAUGGUUUUGA |
| 1951 | GAUUCCGGAUGGUUUUGAA |
| 1952 | AUUCCGGAUGGUUUUGAAU |
| 1953 | UUCCGGAUGGUUUUGAAUG |
| 1954 | UCCGGAUGGUUUUGAAUGA |
| 1955 | CCGGAUGGUUUUGAAUGAA |
| 1956 | CGGAUGGUUUUGAAUGAAG |
| 1957 | GGAUGGUUUUGAAUGAAGG |
| 1958 | GAUGGUUUUGAAUGAAGGG |
| 1959 | AUGGUUUUGAAUGAAGGGC |
| 1960 | UGGUUUUGAAUGAAGGGCC |
| 1961 | GGUUUUGAAUGAAGGGCCU |
| 1962 | GUUUUGAAUGAAGGGCCUG |
| 1963 | UUUUGAAUGAAGGGCCUGA |
| 1964 | UUUGAAUGAAGGGCCUGAG |
| 1965 | UUGAAUGAAGGGCCUGAGG |
| 1966 | UGAAUGAAGGGCCUGAGGG |
| 1967 | GAAUGAAGGGCCUGAGGGU |
| 1968 | AAUGAAGGGCCUGAGGGUG |
| 1969 | AUGAAGGGCCUGAGGGUGG |
| 1970 | UGAAGGGCCUGAGGGUGGG |
| 1971 | GAAGGGCCUGAGGGUGGGC |
| 1972 | AAGGGCCUGAGGGUGGGCA |
| 1973 | AGGGCCUGAGGGUGGGCAG |
| 1974 | GGGCCUGAGGGUGGGCAGU |
| 1975 | GGCCUGAGGGUGGGCAGUC |
| 1976 | GCCUGAGGGUGGGCAGUCU |
| 1977 | CCUGAGGGUGGGCAGUCUG |
| 1978 | CUGAGGGUGGGCAGUCUGU |
| 1979 | UGAGGGUGGGCAGUCUGUC |
| 1980 | GAGGGUGGGCAGUCUGUCU |
| 1981 | AGGGUGGGCAGUCUGUCUA |
| 1982 | GGGUGGGCAGUCUGUCUAU |
| 1983 | GGUGGGCAGUCUGUCUAUC |
| 1984 | GUGGGCAGUCUGUCUAUCA |
| 1985 | UGGGCAGUCUGUCUAUCAU |
| 1986 | GGGCAGUCUGUCUAUCAUG |
| 1987 | GGCAGUCUGUCUAUCAUGU |
| 1988 | GCAGUCUGUCUAUCAUGUA |
| 1989 | CAGUCUGUCUAUCAUGUAC |
| 1990 | AGUCUGUCUAUCAUGUACA |
| 1991 | GUCUGUCUAUCAUGUACAU |
| 1992 | UCUGUCUAUCAUGUACAUC |
| 1993 | CUGUCUAUCAUGUACAUCU |
| 1994 | UGUCUAUCAUGUACAUCUC |
| 1995 | GUCUAUCAUGUACAUCUCC |
| 1996 | UCUAUCAUGUACAUCUCCA |
| 1997 | CUAUCAUGUACAUCUCCAU |
| 1998 | UAUCAUGUACAUCUCCAUA |
| 1999 | AUCAUGUACAUCUCCAUAU |
| 2000 | UCAUGUACAUCUCCAUAUU |
| 2001 | CAUGUACAUCUCCAUAUUC |
| 2002 | AUGUACAUCUCCAUAUUCU |
| 2003 | UGUACAUCUCCAUAUUCUG |
| 2004 | GUACAUCUCCAUAUUCUGG |
| 2005 | UACAUCUCCAUAUUCUGGG |
| 2006 | ACAUCUCCAUAUUCUGGGA |
| 2007 | CAUCUCCAUAUUCUGGGAG |
| 2008 | AUCUCCAUAUUCUGGGAGG |
| 2009 | UCUCCAUAUUCUGGGAGGU |
| 2010 | CUCCAUAUUCUGGGAGGUC |
| 2011 | UCCAUAUUCUGGGAGGUCG |
| 2012 | CCAUAUUCUGGGAGGUCGU |
| 2013 | CAUAUUCUGGGAGGUCGUC |
| 2014 | AUAUUCUGGGAGGUCGUCA |
| 2015 | UAUUCUGGGAGGUCGUCAG |
| 2016 | AUUCUGGGAGGUCGUCAGU |
| 2017 | UUCUGGGAGGUCGUCAGUU |
| 2018 | UCUGGGAGGUCGUCAGUUG |
| 2019 | CUGGGAGGUCGUCAGUUGG |
| 2020 | UGGGAGGUCGUCAGUUGGG |
| 2021 | GGGAGGUCGUCAGUUGGGC |
| 2022 | GGAGGUCGUCAGUUGGGCU |
| 2023 | GAGGUCGUCAGUUGGGCUG |
| 2024 | AGGUCGUCAGUUGGGCUGG |
| 2025 | GGUCGUCAGUUGGGCUGGC |
| 2026 | GUCGUCAGUUGGGCUGGCC |
| 2027 | UCGUCAGUUGGGCUGGCCU |
| 2028 | CGUCAGUUGGGCUGGCCUC |
| 2029 | GUCAGUUGGGCUGGCCUCC |
| 2030 | UCAGUUGGGCUGGCCUCCU |
| 2031 | CAGUUGGGCUGGCCUCCUG |
| 2032 | AGUUGGGCUGGCCUCCUGG |
| 2033 | GUUGGGCUGGCCUCCUGGC |
| 2034 | UUGGGCUGGCCUCCUGGCU |
| 2035 | UGGGCUGGCCUCCUGGCUA |
| 2036 | GGGCUGGCCUCCUGGCUAA |
| 2037 | GGCUGGCCUCCUGGCUAAG |
| 2038 | GCUGGCCUCCUGGCUAAGA |
| 2039 | CUGGCCUCCUGGCUAAGAU |
| 2040 | UGGCCUCCUGGCUAAGAUU |
| 2041 | GGCCUCCUGGCUAAGAUUU |
| 2042 | GCCUCCUGGCUAAGAUUUU |
| 2043 | CCUCCUGGCUAAGAUUUUU |
| 2044 | CUCCUGGCUAAGAUUUUUG |
| 2045 | UCCUGGCUAAGAUUUUUGC |
| 2046 | CCUGGCUAAGAUUUUUGCA |
| 2047 | CUGGCUAAGAUUUUUGCAC |
| 2048 | UGGCUAAGAUUUUUGCACC |
| 2049 | GGCUAAGAUUUUUGCACCA |
| 2050 | GCUAAGAUUUUUGCACCAC |
| 2051 | CUAAGAUUUUUGCACCACA |
| 2052 | UAAGAUUUUUGCACCACAA |
| 2053 | AAGAUUUUUGCACCACAAG |
| 2054 | AGAUUUUUGCACCACAAGA |
| 2055 | GAUUUUUGCACCACAAGAG |
| 2056 | AUUUUUGCACCACAAGAGA |
| 2057 | UUUUUGCACCACAAGAGAU |
| 2058 | UUUUGCACCACAAGAGAUG |
| 2059 | UUUGCACCACAAGAGAUGC |
| 2060 | UUGCACCACAAGAGAUGCU |
| 2061 | UGCACCACAAGAGAUGCUG |
| 2062 | GCACCACAAGAGAUGCUGC |
| 2063 | CACCACAAGAGAUGCUGCA |
| 2064 | ACCACAAGAGAUGCUGCAU |
| 2065 | CCACAAGAGAUGCUGCAUG |
| 2066 | CACAAGAGAUGCUGCAUGU |
| 2067 | ACAAGAGAUGCUGCAUGUG |
| 2068 | CAAGAGAUGCUGCAUGUGU |
| 2069 | AAGAGAUGCUGCAUGUGUA |
| 2070 | AGAGAUGCUGCAUGUGUAC |
| 2071 | GAGAUGCUGCAUGUGUACA |
| 2072 | AGAUGCUGCAUGUGUACAA |
| 2073 | GAUGCUGCAUGUGUACAAA |
| 2074 | AUGCUGCAUGUGUACAAAU |
| 2075 | UGCUGCAUGUGUACAAAUC |
| 2076 | GCUGCAUGUGUACAAAUCA |
| 2077 | CUGCAUGUGUACAAAUCAC |
| 2078 | UGCAUGUGUACAAAUCACU |
| 2079 | GCAUGUGUACAAAUCACUA |
| 2080 | CAUGUGUACAAAUCACUAG |
| 2081 | AUGUGUACAAAUCACUAGC |
| 2082 | UGUGUACAAAUCACUAGCA |
| 2083 | GUGUACAAAUCACUAGCAA |
| 2084 | UGUACAAAUCACUAGCAAA |
| 2085 | GUACAAAUCACUAGCAAAU |
| 2086 | UACAAAUCACUAGCAAAUA |
| 2087 | ACAAAUCACUAGCAAAUAG |
| 2088 | CAAAUCACUAGCAAAUAGA |
| 2089 | AAAUCACUAGCAAAUAGAU |
| 2090 | AAUCACUAGCAAAUAGAUU |
| 2091 | AUCACUAGCAAAUAGAUUU |
| 2092 | UCACUAGCAAAUAGAUUUG |
| 2093 | CACUAGCAAAUAGAUUUGU |
| 2094 | ACUAGCAAAUAGAUUUGUU |
| 2095 | CUAGCAAAUAGAUUUGUUU |
| 2096 | UAGCAAAUAGAUUUGUUUC |
| 2097 | AGCAAAUAGAUUUGUUUCC |
| 2098 | GCAAAUAGAUUUGUUUCCC |
| 2099 | CAAAUAGAUUUGUUUCCCA |
| 2100 | AAAUAGAUUUGUUUCCCAU |
| 2101 | AAUAGAUUUGUUUCCCAUC |
| 2102 | AUAGAUUUGUUUCCCAUCA |
| 2103 | UAGAUUUGUUUCCCAUCAA |
| 2104 | AGAUUUGUUUCCCAUCAAC |
| 2105 | GAUUUGUUUCCCAUCAACU |
| 2106 | AUUUGUUUCCCAUCAACUU |
| 2107 | UUUGUUUCCCAUCAACUUA |
| 2108 | UUGUUUCCCAUCAACUUAG |
| 2109 | UGUUUCCCAUCAACUUAGC |
| 2110 | GUUUCCCAUCAACUUAGCC |
| 2111 | UUUCCCAUCAACUUAGCCA |
| 2112 | UUCCCAUCAACUUAGCCAC |
| 2113 | UCCCAUCAACUUAGCCACU |
| 2114 | CCCAUCAACUUAGCCACUG |
| 2115 | CCAUCAACUUAGCCACUGU |
| 2116 | CAUCAACUUAGCCACUGUU |
| 2117 | AUCAACUUAGCCACUGUUA |
| 2118 | UCAACUUAGCCACUGUUAA |
| 2119 | CAACUUAGCCACUGUUAAU |
| 2120 | AACUUAGCCACUGUUAAUG |
| 2121 | ACUUAGCCACUGUUAAUGU |
| 2122 | CUUAGCCACUGUUAAUGUA |
| 2123 | UUAGCCACUGUUAAUGUAA |
| 2124 | UAGCCACUGUUAAUGUAAA |
| 2125 | AGCCACUGUUAAUGUAAAU |
| 2126 | GCCACUGUUAAUGUAAAUU |
| 2127 | CCACUGUUAAUGUAAAUUG |
| 2128 | CACUGUUAAUGUAAAUUGU |
| 2129 | ACUGUUAAUGUAAAUUGUU |
| 2130 | CUGUUAAUGUAAAUUGUUC |
| 2131 | UGUUAAUGUAAAUUGUUCU |
| 2132 | GUUAAUGUAAAUUGUUCUU |
| 2133 | UUAAUGUAAAUUGUUCUUG |
| 2134 | UAAUGUAAAUUGUUCUUGG |
| 2135 | AAUGUAAAUUGUUCUUGGA |
| 2136 | AUGUAAAUUGUUCUUGGAU |
| 2137 | UGUAAAUUGUUCUUGGAUA |
| 2138 | GUAAAUUGUUCUUGGAUAU |
| 2139 | UAAAUUGUUCUUGGAUAUG |
| 2140 | AAAUUGUUCUUGGAUAUGU |
| 2141 | AAUUGUUCUUGGAUAUGUG |
| 2142 | AUUGUUCUUGGAUAUGUGU |
| 2143 | UUGUUCUUGGAUAUGUGUC |
| 2144 | UGUUCUUGGAUAUGUGUCU |
| 2145 | GUUCUUGGAUAUGUGUCUU |
| 2146 | UUCUUGGAUAUGUGUCUUU |
| 2147 | UCUUGGAUAUGUGUCUUUG |
| 2148 | CUUGGAUAUGUGUCUUUGG |
| 2149 | UUGGAUAUGUGUCUUUGGA |
| 2150 | UGGAUAUGUGUCUUUGGAG |
| 2151 | GGAUAUGUGUCUUUGGAGG |
| 2152 | GAUAUGUGUCUUUGGAGGG |
| 2153 | AUAUGUGUCUUUGGAGGGC |
| 2154 | UAUGUGUCUUUGGAGGGCA |
| 2155 | AUGUGUCUUUGGAGGGCAA |
| 2156 | UGUGUCUUUGGAGGGCAAU |
| 2157 | GUGUCUUUGGAGGGCAAUA |
| 2158 | UGUCUUUGGAGGGCAAUAA |
| 2159 | GUCUUUGGAGGGCAAUAAA |
| 2160 | UCUUUGGAGGGCAAUAAAU |
| 2161 | CUUUGGAGGGCAAUAAAUG |
| 2162 | UUUGGAGGGCAAUAAAUGC |
| 2163 | UUGGAGGGCAAUAAAUGCU |
| 2164 | UGGAGGGCAAUAAAUGCUC |
| 2165 | GGAGGGCAAUAAAUGCUCU |
| 2166 | GAGGGCAAUAAAUGCUCUG |
| 2167 | AGGGCAAUAAAUGCUCUGA |
| 2168 | GGGCAAUAAAUGCUCUGAA |
| 2169 | GGCAAUAAAUGCUCUGAAC |
| 2170 | GCAAUAAAUGCUCUGAACA |
| 2171 | CAAUAAAUGCUCUGAACAG |
| 2172 | AAUAAAUGCUCUGAACAGC |
| 2173 | AUAAAUGCUCUGAACAGCA |
| 2174 | UAAAUGCUCUGAACAGCAC |
| 2175 | AAAUGCUCUGAACAGCACU |
| 2176 | AAUGCUCUGAACAGCACUU |
| 2177 | AUGCUCUGAACAGCACUUG |
| 2178 | UGCUCUGAACAGCACUUGC |
| 2179 | GCUCUGAACAGCACUUGCA |
| 2180 | CUCUGAACAGCACUUGCAC |
| 2181 | UCUGAACAGCACUUGCACA |
| 2182 | CUGAACAGCACUUGCACAA |
| 2183 | UGAACAGCACUUGCACAAU |
| 2184 | GAACAGCACUUGCACAAUA |
| 2185 | CACUUGCACAAUAAAGAUA |
| 2186 | ACUUGCACAAUAAAGAUAC |
| 2187 | CUUGCACAAUAAAGAUACA |
| 2188 | UUGCACAAUAAAGAUACAG |
| 2189 | UGCACAAUAAAGAUACAGC |
| 2190 | GCACAAUAAAGAUACAGCA |
| 2191 | CACAAUAAAGAUACAGCAU |
| 2192 | ACAAUAAAGAUACAGCAUG |
| 2193 | CAAUAAAGAUACAGCAUGU |
| 2194 | AAUAAAGAUACAGCAUGUG |
| 2195 | AUAAAGAUACAGCAUGUGG |
| 2196 | UAAAGAUACAGCAUGUGGA |
| 2197 | AAAGAUACAGCAUGUGGAA |
| 2198 | AAGAUACAGCAUGUGGAAA |
| 2199 | AGAUACAGCAUGUGGAAAA |
| 2200 | GAUACAGCAUGUGGAAAAA |
| 2201 | AUACAGCAUGUGGAAAAAA |
| 2202 | UACAGCAUGUGGAAAAAAA |
| 2203 | ACAGCAUGUGGAAAAAAAA |
| 2204 | CAGCAUGUGGAAAAAAAAA |
| 2205 | AGCAUGUGGAAAAAAAAAA |
| 2206 | GCAUGUGGAAAAAAAAAAA |
| 2207 | CAUGUGGAAAAAAAAAAAA |
| 2208 | AUGUGGAAAAAAAAAAAAA |
| 2209 | UGUGGAAAAAAAAAAAAAA |

TABLE 3
DsRNA molecules targeting mRNA encoding chicken
r-spondin.

| SEQ ID NO | Sequence 5′-3′ |
|---|---|
| 2210 | AUGGAUCUAACAGGCGGCA |
| 2211 | UGGAUCUAACAGGCGGCAG |
| 2212 | GGAUCUAACAGGCGGCAGC |
| 2213 | GAUCUAACAGGCGGCAGCA |
| 2214 | AUCUAACAGGCGGCAGCAA |
| 2215 | UCUAACAGGCGGCAGCAAA |
| 2216 | CUAACAGGCGGCAGCAAAG |
| 2217 | UAACAGGCGGCAGCAAAGU |
| 2218 | AACAGGCGGCAGCAAAGUG |
| 2219 | ACAGGCGGCAGCAAAGUGG |
| 2220 | CAGGCGGCAGCAAAGUGGU |
| 2221 | AGGCGGCAGCAAAGUGGUG |
| 2222 | GGCGGCAGCAAAGUGGUGA |
| 2223 | GCGGCAGCAAAGUGGUGAA |
| 2224 | CGGCAGCAAAGUGGUGAAG |
| 2225 | GGCAGCAAAGUGGUGAAGG |
| 2226 | GCAGCAAAGUGGUGAAGGG |
| 2227 | CAGCAAAGUGGUGAAGGGC |
| 2228 | AGCAAAGUGGUGAAGGGCA |
| 2229 | GCAAAGUGGUGAAGGGCAA |
| 2230 | CAAAGUGGUGAAGGGCAAG |
| 2231 | AAAGUGGUGAAGGGCAAGA |
| 2232 | AAGUGGUGAAGGGCAAGAG |
| 2233 | AGUGGUGAAGGGCAAGAGG |
| 2234 | GUGGUGAAGGGCAAGAGGC |
| 2235 | UGGUGAAGGGCAAGAGGCA |
| 2236 | GGUGAAGGGCAAGAGGCAA |
| 2237 | GUGAAGGGCAAGAGGCAAA |
| 2238 | UGAAGGGCAAGAGGCAAAG |
| 2239 | GAAGGGCAAGAGGCAAAGG |
| 2240 | AAGGGCAAGAGGCAAAGGC |
| 2241 | AGGGCAAGAGGCAAAGGCG |
| 2242 | GGGCAAGAGGCAAAGGCGA |
| 2243 | GGCAAGAGGCAAAGGCGAA |
| 2244 | GCAAGAGGCAAAGGCGAAU |
| 2245 | CAAGAGGCAAAGGCGAAUU |
| 2246 | AAGAGGCAAAGGCGAAUUA |
| 2247 | AGAGGCAAAGGCGAAUUAG |
| 2248 | GAGGCAAAGGCGAAUUAGC |
| 2249 | AGGCAAAGGCGAAUUAGCA |
| 2250 | GGCAAAGGCGAAUUAGCAC |
| 2251 | GCAAAGGCGAAUUAGCACU |
| 2252 | CAAAGGCGAAUUAGCACUG |
| 2253 | AAAGGCGAAUUAGCACUGA |
| 2254 | AAGGCGAAUUAGCACUGAG |
| 2255 | AGGCGAAUUAGCACUGAGC |
| 2256 | GGCGAAUUAGCACUGAGCU |
| 2257 | GCGAAUUAGCACUGAGCUG |
| 2258 | CGAAUUAGCACUGAGCUGA |
| 2259 | GAAUUAGCACUGAGCUGAG |
| 2260 | AAUUAGCACUGAGCUGAGC |
| 2261 | AUUAGCACUGAGCUGAGCC |
| 2262 | UUAGCACUGAGCUGAGCCA |
| 2263 | UAGCACUGAGCUGAGCCAG |
| 2264 | AGCACUGAGCUGAGCCAGG |
| 2265 | GCACUGAGCUGAGCCAGGG |
| 2266 | CACUGAGCUGAGCCAGGGC |
| 2267 | ACUGAGCUGAGCCAGGGCU |
| 2268 | CUGAGCUGAGCCAGGGCUG |
| 2269 | UGAGCUGAGCCAGGGCUGU |
| 2270 | GAGCUGAGCCAGGGCUGUG |
| 2271 | AGCUGAGCCAGGGCUGUGC |
| 2272 | GCUGAGCCAGGGCUGUGCC |
| 2273 | CUGAGCCAGGGCUGUGCCA |
| 2274 | UGAGCCAGGGCUGUGCCAG |
| 2275 | GAGCCAGGGCUGUGCCAGG |
| 2276 | AGCCAGGGCUGUGCCAGGG |
| 2277 | GCCAGGGCUGUGCCAGGGG |
| 2278 | CCAGGGCUGUGCCAGGGGC |
| 2279 | CAGGGCUGUGCCAGGGGCU |
| 2280 | AGGGCUGUGCCAGGGGCUG |
| 2281 | GGGCUGUGCCAGGGGCUGC |
| 2282 | GGCUGUGCCAGGGGCUGCG |
| 2283 | GCUGUGCCAGGGGCUGCGA |
| 2284 | CUGUGCCAGGGGCUGCGAC |
| 2285 | UGUGCCAGGGGCUGCGACC |
| 2286 | GUGCCAGGGGCUGCGACCU |
| 2287 | UGCCAGGGGCUGCGACCUG |
| 2288 | GCCAGGGGCUGCGACCUGU |
| 2289 | CCAGGGGCUGCGACCUGUG |
| 2290 | CAGGGGCUGCGACCUGUGC |
| 2291 | AGGGGCUGCGACCUGUGCU |
| 2292 | GGGGCUGCGACCUGUGCUC |
| 2293 | GGGCUGCGACCUGUGCUCU |
| 2294 | GGCUGCGACCUGUGCUCUG |
| 2295 | GCUGCGACCUGUGCUCUGA |
| 2296 | CUGCGACCUGUGCUCUGAG |
| 2297 | UGCGACCUGUGCUCUGAGU |
| 2298 | GCGACCUGUGCUCUGAGUU |
| 2299 | CGACCUGUGCUCUGAGUUC |
| 2300 | GACCUGUGCUCUGAGUUCA |
| 2301 | ACCUGUGCUCUGAGUUCAA |
| 2302 | CCUGUGCUCUGAGUUCAAC |
| 2303 | CUGUGCUCUGAGUUCAACG |
| 2304 | UGUGCUCUGAGUUCAACGG |
| 2305 | GUGCUCUGAGUUCAACGGG |
| 2306 | UGCUCUGAGUUCAACGGGU |
| 2307 | GCUCUGAGUUCAACGGGUG |
| 2308 | CUCUGAGUUCAACGGGUGC |
| 2309 | UCUGAGUUCAACGGGUGCC |
| 2310 | CUGAGUUCAACGGGUGCCU |
| 2311 | UGAGUUCAACGGGUGCCUG |
| 2312 | GAGUUCAACGGGUGCCUGA |
| 2313 | AGUUCAACGGGUGCCUGAG |
| 2314 | GUUCAACGGGUGCCUGAGA |
| 2315 | UUCAACGGGUGCCUGAGAU |
| 2316 | UCAACGGGUGCCUGAGAUG |
| 2317 | CAACGGGUGCCUGAGAUGU |
| 2318 | AACGGGUGCCUGAGAUGUU |
| 2319 | ACGGGUGCCUGAGAUGUUC |
| 2320 | CGGGUGCCUGAGAUGUUCC |
| 2321 | GGGUGCCUGAGAUGUUCCC |
| 2322 | GGUGCCUGAGAUGUUCCCC |
| 2323 | GUGCCUGAGAUGUUCCCCC |
| 2324 | UGCCUGAGAUGUUCCCCCA |
| 2325 | GCCUGAGAUGUUCCCCCAA |
| 2326 | CCUGAGAUGUUCCCCCAAG |
| 2327 | CUGAGAUGUUCCCCCAAGC |
| 2328 | UGAGAUGUUCCCCCAAGCU |
| 2329 | GAGAUGUUCCCCCAAGCUC |
| 2330 | AGAUGUUCCCCCAAGCUCU |
| 2331 | GAUGUUCCCCCAAGCUCUU |
| 2332 | AUGUUCCCCCAAGCUCUUC |
| 2333 | UGUUCCCCCAAGCUCUUCA |
| 2334 | GUUCCCCCAAGCUCUUCAU |
| 2335 | UUCCCCCAAGCUCUUCAUC |
| 2336 | UCCCCCAAGCUCUUCAUCC |
| 2337 | CCCCCAAGCUCUUCAUCCU |
| 2338 | CCCCAAGCUCUUCAUCCUU |
| 2339 | CCCAAGCUCUUCAUCCUUC |
| 2340 | CCAAGCUCUUCAUCCUUCU |
| 2341 | CAAGCUCUUCAUCCUUCUG |
| 2342 | AAGCUCUUCAUCCUUCUGG |
| 2343 | AGCUCUUCAUCCUUCUGGA |
| 2344 | GCUCUUCAUCCUUCUGGAG |
| 2345 | CUCUUCAUCCUUCUGGAGA |
| 2346 | UCUUCAUCCUUCUGGAGAG |
| 2347 | CUUCAUCCUUCUGGAGAGG |
| 2348 | UUCAUCCUUCUGGAGAGGA |
| 2349 | UCAUCCUUCUGGAGAGGAA |
| 2350 | CAUCCUUCUGGAGAGGAAC |
| 2351 | AUCCUUCUGGAGAGGAACG |
| 2352 | UCCUUCUGGAGAGGAACGA |
| 2353 | CCUUCUGGAGAGGAACGAU |
| 2354 | CUUCUGGAGAGGAACGAUA |
| 2355 | UUCUGGAGAGGAACGAUAU |
| 2356 | UCUGGAGAGGAACGAUAUC |
| 2357 | CUGGAGAGGAACGAUAUCC |
| 2358 | UGGAGAGGAACGAUAUCCG |
| 2359 | GGAGAGGAACGAUAUCCGG |
| 2360 | GAGAGGAACGAUAUCCGGC |
| 2361 | AGAGGAACGAUAUCCGGCA |
| 2362 | GAGGAACGAUAUCCGGCAA |
| 2363 | AGGAACGAUAUCCGGCAAA |
| 2364 | GGAACGAUAUCCGGCAAAU |
| 2365 | GAACGAUAUCCGGCAAAUU |
| 2366 | AACGAUAUCCGGCAAAUUG |
| 2367 | ACGAUAUCCGGCAAAUUGG |
| 2368 | CGAUAUCCGGCAAAUUGGG |
| 2369 | GAUAUCCGGCAAAUUGGGA |
| 2370 | AUAUCCGGCAAAUUGGGAU |
| 2371 | UAUCCGGCAAAUUGGGAUC |
| 2372 | AUCCGGCAAAUUGGGAUCU |
| 2373 | UCCGGCAAAUUGGGAUCUG |
| 2374 | CCGGCAAAUUGGGAUCUGC |
| 2375 | CGGCAAAUUGGGAUCUGCC |
| 2376 | GGCAAAUUGGGAUCUGCCU |
| 2377 | GCAAAUUGGGAUCUGCCUC |
| 2378 | CAAAUUGGGAUCUGCCUCC |
| 2379 | AAAUUGGGAUCUGCCUCCC |
| 2380 | AAUUGGGAUCUGCCUCCCA |
| 2381 | AUUGGGAUCUGCCUCCCAU |
| 2382 | UUGGGAUCUGCCUCCCAUC |
| 2383 | UGGGAUCUGCCUCCCAUCC |
| 2384 | GGGAUCUGCCUCCCAUCCU |
| 2385 | GGAUCUGCCUCCCAUCCUG |
| 2386 | GAUCUGCCUCCCAUCCUGU |
| 2387 | AUCUGCCUCCCAUCCUGUC |
| 2388 | UCUGCCUCCCAUCCUGUCC |
| 2389 | CUGCCUCCCAUCCUGUCCA |
| 2390 | UGCCUCCCAUCCUGUCCAC |
| 2391 | GCCUCCCAUCCUGUCCACU |
| 2392 | CCUCCCAUCCUGUCCACUG |
| 2393 | CUCCCAUCCUGUCCACUGG |
| 2394 | UCCCAUCCUGUCCACUGGG |
| 2395 | CCCAUCCUGUCCACUGGGA |
| 2396 | CCAUCCUGUCCACUGGGAU |
| 2397 | CAUCCUGUCCACUGGGAUA |
| 2398 | AUCCUGUCCACUGGGAUAC |
| 2399 | UCCUGUCCACUGGGAUACU |
| 2400 | CCUGUCCACUGGGAUACUU |
| 2401 | CUGUCCACUGGGAUACUUU |
| 2402 | UGUCCACUGGGAUACUUUG |
| 2403 | GUCCACUGGGAUACUUUGG |
| 2404 | UCCACUGGGAUACUUUGGC |
| 2405 | CCACUGGGAUACUUUGGCC |
| 2406 | CACUGGGAUACUUUGGCCU |
| 2407 | ACUGGGAUACUUUGGCCUU |
| 2408 | CUGGGAUACUUUGGCCUUC |
| 2409 | UGGGAUACUUUGGCCUUCG |
| 2410 | GGGAUACUUUGGCCUUCGC |
| 2411 | GGAUACUUUGGCCUUCGCA |
| 2412 | GAUACUUUGGCCUUCGCAA |
| 2413 | AUACUUUGGCCUUCGCAAU |
| 2414 | UACUUUGGCCUUCGCAAUA |
| 2415 | ACUUUGGCCUUCGCAAUAC |
| 2416 | CUUUGGCCUUCGCAAUACA |
| 2417 | UUUGGCCUUCGCAAUACAG |
| 2418 | UUGGCCUUCGCAAUACAGA |
| 2419 | UGGCCUUCGCAAUACAGAC |
| 2420 | GGCCUUCGCAAUACAGACA |
| 2421 | GCCUUCGCAAUACAGACAU |
| 2422 | CCUUCGCAAUACAGACAUG |
| 2423 | CUUCGCAAUACAGACAUGA |
| 2424 | UUCGCAAUACAGACAUGAA |
| 2425 | UCGCAAUACAGACAUGAAC |
| 2426 | CGCAAUACAGACAUGAACA |
| 2427 | GCAAUACAGACAUGAACAA |
| 2428 | CAAUACAGACAUGAACAAG |
| 2429 | AAUACAGACAUGAACAAGU |
| 2430 | AUACAGACAUGAACAAGUG |
| 2431 | UACAGACAUGAACAAGUGC |
| 2432 | ACAGACAUGAACAAGUGCA |
| 2433 | CAGACAUGAACAAGUGCAU |
| 2434 | AGACAUGAACAAGUGCAUC |
| 2435 | GACAUGAACAAGUGCAUCA |
| 2436 | ACAUGAACAAGUGCAUCAA |
| 2437 | CAUGAACAAGUGCAUCAAA |
| 2438 | AUGAACAAGUGCAUCAAAU |
| 2439 | UGAACAAGUGCAUCAAAUG |
| 2440 | GAACAAGUGCAUCAAAUGC |
| 2441 | AACAAGUGCAUCAAAUGCA |
| 2442 | ACAAGUGCAUCAAAUGCAA |
| 2443 | CAAGUGCAUCAAAUGCAAA |
| 2444 | AAGUGCAUCAAAUGCAAAA |
| 2445 | AGUGCAUCAAAUGCAAAAU |
| 2446 | GUGCAUCAAAUGCAAAAUC |
| 2447 | UGCAUCAAAUGCAAAAUCG |
| 2448 | GCAUCAAAUGCAAAAUCGA |
| 2449 | CAUCAAAUGCAAAAUCGAG |
| 2450 | AUCAAAUGCAAAAUCGAGA |
| 2451 | UCAAAUGCAAAAUCGAGAA |
| 2452 | CAAAUGCAAAAUCGAGAAC |
| 2453 | AAAUGCAAAAUCGAGAACU |
| 2454 | AAUGCAAAAUCGAGAACUG |
| 2455 | AUGCAAAAUCGAGAACUGU |
| 2456 | UGCAAAAUCGAGAACUGUG |
| 2457 | GCAAAAUCGAGAACUGUGA |
| 2458 | CAAAAUCGAGAACUGUGAG |
| 2459 | AAAAUCGAGAACUGUGAGU |
| 2460 | AAAUCGAGAACUGUGAGUC |
| 2461 | AAUCGAGAACUGUGAGUCC |
| 2462 | AUCGAGAACUGUGAGUCCU |
| 2463 | UCGAGAACUGUGAGUCCUG |
| 2464 | CGAGAACUGUGAGUCCUGC |
| 2465 | GAGAACUGUGAGUCCUGCU |
| 2466 | AGAACUGUGAGUCCUGCUU |
| 2467 | GAACUGUGAGUCCUGCUUC |
| 2468 | AACUGUGAGUCCUGCUUCA |
| 2469 | ACUGUGAGUCCUGCUUCAG |
| 2470 | CUGUGAGUCCUGCUUCAGC |
| 2471 | UGUGAGUCCUGCUUCAGCC |
| 2472 | GUGAGUCCUGCUUCAGCCG |
| 2473 | UGAGUCCUGCUUCAGCCGA |
| 2474 | GAGUCCUGCUUCAGCCGAA |
| 2475 | AGUCCUGCUUCAGCCGAAA |
| 2476 | GUCCUGCUUCAGCCGAAAC |
| 2477 | UCCUGCUUCAGCCGAAACU |
| 2478 | CCUGCUUCAGCCGAAACUU |
| 2479 | CUGCUUCAGCCGAAACUUU |
| 2480 | UGCUUCAGCCGAAACUUUU |
| 2481 | GCUUCAGCCGAAACUUUUG |
| 2482 | CUUCAGCCGAAACUUUUGC |
| 2483 | UUCAGCCGAAACUUUUGCA |
| 2484 | UCAGCCGAAACUUUUGCAC |
| 2485 | CAGCCGAAACUUUUGCACA |
| 2486 | AGCCGAAACUUUUGCACAA |
| 2487 | GCCGAAACUUUUGCACAAA |
| 2488 | CCGAAACUUUUGCACAAAA |
| 2489 | CGAAACUUUUGCACAAAAU |
| 2490 | GAAACUUUUGCACAAAAUG |
| 2491 | AAACUUUUGCACAAAAUGU |
| 2492 | AACUUUUGCACAAAAUGUA |
| 2493 | ACUUUUGCACAAAAUGUAA |
| 2494 | CUUUUGCACAAAAUGUAAG |
| 2495 | UUUUGCACAAAAUGUAAGG |
| 2496 | UUUGCACAAAAUGUAAGGA |
| 2497 | UUGCACAAAAUGUAAGGAA |
| 2498 | UGCACAAAAUGUAAGGAAG |
| 2499 | GCACAAAAUGUAAGGAAGG |
| 2500 | CACAAAAUGUAAGGAAGGU |
| 2501 | ACAAAAUGUAAGGAAGGUU |
| 2502 | CAAAAUGUAAGGAAGGUUU |
| 2503 | AAAAUGUAAGGAAGGUUUG |
| 2504 | AAAUGUAAGGAAGGUUUGU |
| 2505 | AAUGUAAGGAAGGUUUGUA |
| 2506 | AUGUAAGGAAGGUUUGUAU |
| 2507 | UGUAAGGAAGGUUUGUAUU |
| 2508 | GUAAGGAAGGUUUGUAUUU |
| 2509 | UAAGGAAGGUUUGUAUUUG |
| 2510 | AAGGAAGGUUUGUAUUUGC |
| 2511 | AGGAAGGUUUGUAUUUGCA |
| 2512 | GGAAGGUUUGUAUUUGCAC |
| 2513 | GAAGGUUUGUAUUUGCACA |
| 2514 | AAGGUUUGUAUUUGCACAA |
| 2515 | AGGUUUGUAUUUGCACAAA |
| 2516 | GGUUUGUAUUUGCACAAAG |
| 2517 | GUUUGUAUUUGCACAAAGG |
| 2518 | UUUGUAUUUGCACAAAGGG |
| 2519 | UUGUAUUUGCACAAAGGGA |
| 2520 | UGUAUUUGCACAAAGGGAG |
| 2521 | GUAUUUGCACAAAGGGAGA |
| 2522 | UAUUUGCACAAAGGGAGAU |
| 2523 | AUUUGCACAAAGGGAGAUG |
| 2524 | UUUGCACAAAGGGAGAUGU |
| 2525 | UUGCACAAAGGGAGAUGUU |
| 2526 | UGCACAAAGGGAGAUGUUA |
| 2527 | GCACAAAGGGAGAUGUUAC |
| 2528 | CACAAAGGGAGAUGUUACG |
| 2529 | ACAAAGGGAGAUGUUACGU |
| 2530 | CAAAGGGAGAUGUUACGUC |
| 2531 | AAAGGGAGAUGUUACGUCA |
| 2532 | AAGGGAGAUGUUACGUCAC |
| 2533 | AGGGAGAUGUUACGUCACG |
| 2534 | GGGAGAUGUUACGUCACGU |
| 2535 | GGAGAUGUUACGUCACGUG |
| 2536 | GAGAUGUUACGUCACGUGC |
| 2537 | AGAUGUUACGUCACGUGCC |
| 2538 | GAUGUUACGUCACGUGCCC |
| 2539 | AUGUUACGUCACGUGCCCC |
| 2540 | UGUUACGUCACGUGCCCCG |
| 2541 | GUUACGUCACGUGCCCCGA |
| 2542 | UUACGUCACGUGCCCCGAA |
| 2543 | UACGUCACGUGCCCCGAAG |
| 2544 | ACGUCACGUGCCCCGAAGG |
| 2545 | CGUCACGUGCCCCGAAGGC |
| 2546 | GUCACGUGCCCCGAAGGCU |
| 2547 | UCACGUGCCCCGAAGGCUA |
| 2548 | CACGUGCCCCGAAGGCUAC |
| 2549 | ACGUGCCCCGAAGGCUACU |
| 2550 | CGUGCCCCGAAGGCUACUC |
| 2551 | GUGCCCCGAAGGCUACUCU |
| 2552 | UGCCCCGAAGGCUACUCUG |
| 2553 | GCCCCGAAGGCUACUCUGC |
| 2554 | CCCCGAAGGCUACUCUGCU |
| 2555 | CCCGAAGGCUACUCUGCUG |
| 2556 | CCGAAGGCUACUCUGCUGC |
| 2557 | CGAAGGCUACUCUGCUGCC |
| 2558 | GAAGGCUACUCUGCUGCCA |
| 2559 | AAGGCUACUCUGCUGCCAA |
| 2560 | AGGCUACUCUGCUGCCAAU |
| 2561 | GGCUACUCUGCUGCCAAUG |
| 2562 | GCUACUCUGCUGCCAAUGG |
| 2563 | CUACUCUGCUGCCAAUGGC |
| 2564 | UACUCUGCUGCCAAUGGCA |
| 2565 | ACUCUGCUGCCAAUGGCAC |
| 2566 | CUCUGCUGCCAAUGGCACC |
| 2567 | UCUGCUGCCAAUGGCACCA |
| 2568 | CUGCUGCCAAUGGCACCAU |
| 2569 | UGCUGCCAAUGGCACCAUG |
| 2570 | GCUGCCAAUGGCACCAUGG |
| 2571 | CUGCCAAUGGCACCAUGGA |
| 2572 | UGCCAAUGGCACCAUGGAG |
| 2573 | GCCAAUGGCACCAUGGAGU |
| 2574 | CCAAUGGCACCAUGGAGUG |
| 2575 | CAAUGGCACCAUGGAGUGC |
| 2576 | AAUGGCACCAUGGAGUGCA |
| 2577 | AUGGCACCAUGGAGUGCAG |
| 2578 | UGGCACCAUGGAGUGCAGC |
| 2579 | GGCACCAUGGAGUGCAGCA |
| 2580 | GCACCAUGGAGUGCAGCAG |
| 2581 | CACCAUGGAGUGCAGCAGU |
| 2582 | ACCAUGGAGUGCAGCAGUC |
| 2583 | CCAUGGAGUGCAGCAGUCC |
| 2584 | CAUGGAGUGCAGCAGUCCU |
| 2585 | AUGGAGUGCAGCAGUCCUG |
| 2586 | UGGAGUGCAGCAGUCCUGC |
| 2587 | GGAGUGCAGCAGUCCUGCG |
| 2588 | GAGUGCAGCAGUCCUGCGC |
| 2589 | AGUGCAGCAGUCCUGCGCA |
| 2590 | GUGCAGCAGUCCUGCGCAA |
| 2591 | UGCAGCAGUCCUGCGCAAU |
| 2592 | GCAGCAGUCCUGCGCAAUG |
| 2593 | CAGCAGUCCUGCGCAAUGU |
| 2594 | AGCAGUCCUGCGCAAUGUG |
| 2595 | GCAGUCCUGCGCAAUGUGA |
| 2596 | CAGUCCUGCGCAAUGUGAA |
| 2597 | AGUCCUGCGCAAUGUGAAA |
| 2598 | GUCCUGCGCAAUGUGAAAU |
| 2599 | UCCUGCGCAAUGUGAAAUG |
| 2600 | CCUGCGCAAUGUGAAAUGA |
| 2601 | CUGCGCAAUGUGAAAUGAG |
| 2602 | UGCGCAAUGUGAAAUGAGU |
| 2603 | GCGCAAUGUGAAAUGAGUG |
| 2604 | CGCAAUGUGAAAUGAGUGA |
| 2605 | GCAAUGUGAAAUGAGUGAG |
| 2606 | CAAUGUGAAAUGAGUGAGU |
| 2607 | AAUGUGAAAUGAGUGAGUG |
| 2608 | AUGUGAAAUGAGUGAGUGG |
| 2609 | UGUGAAAUGAGUGAGUGGG |
| 2610 | GUGAAAUGAGUGAGUGGGG |
| 2611 | UGAAAUGAGUGAGUGGGGG |
| 2612 | GAAAUGAGUGAGUGGGGGC |
| 2613 | AAAUGAGUGAGUGGGGGCC |
| 2614 | AAUGAGUGAGUGGGGGCCC |
| 2615 | AUGAGUGAGUGGGGGCCCU |
| 2616 | UGAGUGAGUGGGGGCCCUG |
| 2617 | GAGUGAGUGGGGGCCCUGG |
| 2618 | AGUGAGUGGGGGCCCUGGG |
| 2619 | GUGAGUGGGGGCCCUGGGG |
| 2620 | UGAGUGGGGGCCCUGGGGG |
| 2621 | GAGUGGGGGCCCUGGGGGC |
| 2622 | AGUGGGGGCCCUGGGGGCC |
| 2623 | GUGGGGGCCCUGGGGGCCC |
| 2624 | UGGGGGCCCUGGGGGCCCU |
| 2625 | GGGGGCCCUGGGGGCCCUG |
| 2626 | GGGGCCCUGGGGGCCCUGC |
| 2627 | GGGCCCUGGGGGCCCUGCU |
| 2628 | GGCCCUGGGGGCCCUGCUC |
| 2629 | GCCCUGGGGGCCCUGCUCC |
| 2630 | CCCUGGGGGCCCUGCUCCA |
| 2631 | CCUGGGGGCCCUGCUCCAA |
| 2632 | CUGGGGGCCCUGCUCCAAG |
| 2633 | UGGGGGCCCUGCUCCAAGA |
| 2634 | GGGGGCCCUGCUCCAAGAA |
| 2635 | GGGGCCCUGCUCCAAGAAG |
| 2636 | GGGCCCUGCUCCAAGAAGA |
| 2637 | GGCCCUGCUCCAAGAAGAG |
| 2638 | GCCCUGCUCCAAGAAGAGG |
| 2639 | CCCUGCUCCAAGAAGAGGA |
| 2640 | CCUGCUCCAAGAAGAGGAA |
| 2641 | CUGCUCCAAGAAGAGGAAG |
| 2642 | UGCUCCAAGAAGAGGAAGC |
| 2643 | GCUCCAAGAAGAGGAAGCU |
| 2644 | CUCCAAGAAGAGGAAGCUG |
| 2645 | UCCAAGAAGAGGAAGCUGU |
| 2646 | CCAAGAAGAGGAAGCUGUG |
| 2647 | CAAGAAGAGGAAGCUGUGU |
| 2648 | AAGAAGAGGAAGCUGUGUG |
| 2649 | AGAAGAGGAAGCUGUGUGG |
| 2650 | GAAGAGGAAGCUGUGUGGC |
| 2651 | AAGAGGAAGCUGUGUGGCU |
| 2652 | AGAGGAAGCUGUGUGGCUU |
| 2653 | GAGGAAGCUGUGUGGCUUC |
| 2654 | AGGAAGCUGUGUGGCUUCA |
| 2655 | GGAAGCUGUGUGGCUUCAA |
| 2656 | GAAGCUGUGUGGCUUCAAG |
| 2657 | AAGCUGUGUGGCUUCAAGA |
| 2658 | AGCUGUGUGGCUUCAAGAA |
| 2659 | GCUGUGUGGCUUCAAGAAG |
| 2660 | CUGUGUGGCUUCAAGAAGG |
| 2661 | UGUGUGGCUUCAAGAAGGG |
| 2662 | GUGUGGCUUCAAGAAGGGG |
| 2663 | UGUGGCUUCAAGAAGGGGA |
| 2664 | GUGGCUUCAAGAAGGGGAA |
| 2665 | UGGCUUCAAGAAGGGGAAC |
| 2666 | GGCUUCAAGAAGGGGAACG |
| 2667 | GCUUCAAGAAGGGGAACGA |
| 2668 | CUUCAAGAAGGGGAACGAG |
| 2669 | UUCAAGAAGGGGAACGAGG |
| 2670 | UCAAGAAGGGGAACGAGGA |
| 2671 | CAAGAAGGGGAACGAGGAC |
| 2672 | AAGAAGGGGAACGAGGACC |
| 2673 | AGAAGGGGAACGAGGACCG |
| 2674 | GAAGGGGAACGAGGACCGA |
| 2675 | AAGGGGAACGAGGACCGAA |
| 2676 | AGGGGAACGAGGACCGAAC |
| 2677 | GGGGAACGAGGACCGAACG |
| 2678 | GGGAACGAGGACCGAACGC |
| 2679 | GGAACGAGGACCGAACGCG |
| 2680 | GAACGAGGACCGAACGCGG |
| 2681 | AACGAGGACCGAACGCGGC |
| 2682 | ACGAGGACCGAACGCGGCG |
| 2683 | CGAGGACCGAACGCGGCGG |
| 2684 | GAGGACCGAACGCGGCGGA |
| 2685 | AGGACCGAACGCGGCGGAU |
| 2686 | GGACCGAACGCGGCGGAUC |
| 2687 | GACCGAACGCGGCGGAUCC |
| 2688 | ACCGAACGCGGCGGAUCCU |
| 2689 | CCGAACGCGGCGGAUCCUG |
| 2690 | CGAACGCGGCGGAUCCUGC |
| 2691 | GAACGCGGCGGAUCCUGCA |
| 2692 | AACGCGGCGGAUCCUGCAG |
| 2693 | ACGCGGCGGAUCCUGCAGG |
| 2694 | CGCGGCGGAUCCUGCAGGC |
| 2695 | GCGGCGGAUCCUGCAGGCU |
| 2696 | CGGCGGAUCCUGCAGGCUC |
| 2697 | GGCGGAUCCUGCAGGCUCC |
| 2698 | GCGGAUCCUGCAGGCUCCC |
| 2699 | CGGAUCCUGCAGGCUCCCU |
| 2700 | GGAUCCUGCAGGCUCCCUC |
| 2701 | GAUCCUGCAGGCUCCCUCU |
| 2702 | AUCCUGCAGGCUCCCUCUG |
| 2703 | UCCUGCAGGCUCCCUCUGG |
| 2704 | CCUGCAGGCUCCCUCUGGG |
| 2705 | CUGCAGGCUCCCUCUGGGG |
| 2706 | UGCAGGCUCCCUCUGGGGA |
| 2707 | GCAGGCUCCCUCUGGGGAC |
| 2708 | CAGGCUCCCUCUGGGGACG |
| 2709 | AGGCUCCCUCUGGGGACGU |
| 2710 | GGCUCCCUCUGGGGACGUG |
| 2711 | GCUCCCUCUGGGGACGUGU |
| 2712 | CUCCCUCUGGGGACGUGUC |
| 2713 | UCCCUCUGGGGACGUGUCC |
| 2714 | CCCUCUGGGGACGUGUCCC |
| 2715 | CCUCUGGGGACGUGUCCCU |
| 2716 | CUCUGGGGACGUGUCCCUG |
| 2717 | UCUGGGGACGUGUCCCUGU |
| 2718 | CUGGGGACGUGUCCCUGUG |
| 2719 | UGGGGACGUGUCCCUGUGC |
| 2720 | GGGGACGUGUCCCUGUGCC |
| 2721 | GGGACGUGUCCCUGUGCCC |
| 2722 | GGACGUGUCCCUGUGCCCC |
| 2723 | GACGUGUCCCUGUGCCCCG |
| 2724 | ACGUGUCCCUGUGCCCCGC |
| 2725 | CGUGUCCCUGUGCCCCGCC |
| 2726 | GUGUCCCUGUGCCCCGCCA |
| 2727 | UGUCCCUGUGCCCCGCCAC |
| 2728 | GUCCCUGUGCCCCGCCACC |
| 2729 | UCCCUGUGCCCCGCCACCA |
| 2730 | CCCUGUGCCCCGCCACCAC |
| 2731 | CCUGUGCCCCGCCACCACG |
| 2732 | CUGUGCCCCGCCACCACGG |
| 2733 | UGUGCCCCGCCACCACGGA |
| 2734 | GUGCCCCGCCACCACGGAG |
| 2735 | UGCCCCGCCACCACGGAGG |
| 2736 | GCCCCGCCACCACGGAGGU |
| 2737 | CCCCGCCACCACGGAGGUG |
| 2738 | CCCGCCACCACGGAGGUGC |
| 2739 | CCGCCACCACGGAGGUGCG |
| 2740 | CGCCACCACGGAGGUGCGC |
| 2741 | GCCACCACGGAGGUGCGCA |
| 2742 | CCACCACGGAGGUGCGCAG |
| 2743 | CACCACGGAGGUGCGCAGA |
| 2744 | ACCACGGAGGUGCGCAGAU |
| 2745 | CCACGGAGGUGCGCAGAUG |
| 2746 | CACGGAGGUGCGCAGAUGC |
| 2747 | ACGGAGGUGCGCAGAUGCA |
| 2748 | CGGAGGUGCGCAGAUGCAC |
| 2749 | GGAGGUGCGCAGAUGCACU |
| 2750 | GAGGUGCGCAGAUGCACUG |
| 2751 | AGGUGCGCAGAUGCACUGU |
| 2752 | GGUGCGCAGAUGCACUGUG |
| 2753 | GUGCGCAGAUGCACUGUGC |
| 2754 | UGCGCAGAUGCACUGUGCA |
| 2755 | GCGCAGAUGCACUGUGCAG |
| 2756 | CGCAGAUGCACUGUGCAGA |
| 2757 | GCAGAUGCACUGUGCAGAA |
| 2758 | CAGAUGCACUGUGCAGAAG |
| 2759 | AGAUGCACUGUGCAGAAGA |
| 2760 | GAUGCACUGUGCAGAAGAG |
| 2761 | AUGCACUGUGCAGAAGAGC |
| 2762 | UGCACUGUGCAGAAGAGCC |
| 2763 | GCACUGUGCAGAAGAGCCA |
| 2764 | CACUGUGCAGAAGAGCCAA |
| 2765 | ACUGUGCAGAAGAGCCAAU |
| 2766 | CUGUGCAGAAGAGCCAAUG |
| 2767 | UGUGCAGAAGAGCCAAUGC |
| 2768 | GUGCAGAAGAGCCAAUGCC |
| 2769 | UGCAGAAGAGCCAAUGCCC |
| 2770 | GCAGAAGAGCCAAUGCCCC |
| 2771 | CAGAAGAGCCAAUGCCCCG |
| 2772 | AGAAGAGCCAAUGCCCCGA |
| 2773 | GAAGAGCCAAUGCCCCGAA |
| 2774 | AAGAGCCAAUGCCCCGAAG |
| 2775 | AGAGCCAAUGCCCCGAAGG |
| 2776 | GAGCCAAUGCCCCGAAGGG |
| 2777 | AGCCAAUGCCCCGAAGGGA |
| 2778 | GCCAAUGCCCCGAAGGGAA |
| 2779 | CCAAUGCCCCGAAGGGAAA |
| 2780 | CAAUGCCCCGAAGGGAAAA |
| 2781 | AAUGCCCCGAAGGGAAAAG |
| 2782 | AUGCCCCGAAGGGAAAAGG |
| 2783 | UGCCCCGAAGGGAAAAGGA |
| 2784 | GCCCCGAAGGGAAAAGGAA |
| 2785 | CCCCGAAGGGAAAAGGAAG |
| 2786 | CCCGAAGGGAAAAGGAAGA |
| 2787 | CCGAAGGGAAAAGGAAGAA |
| 2788 | CGAAGGGAAAAGGAAGAAA |
| 2789 | GAAGGGAAAAGGAAGAAAA |
| 2790 | AAGGGAAAAGGAAGAAAAA |
| 2791 | AGGGAAAAGGAAGAAAAAG |
| 2792 | GGGAAAAGGAAGAAAAAGG |
| 2793 | GGAAAAGGAAGAAAAAGGA |
| 2794 | GAAAAGGAAGAAAAAGGAC |
| 2795 | AAAAGGAAGAAAAAGGACG |
| 2796 | AAAGGAAGAAAAAGGACGA |
| 2797 | AAGGAAGAAAAAGGACGAG |
| 2798 | AGGAAGAAAAAGGACGAGC |
| 2799 | GGAAGAAAAAGGACGAGCA |
| 2800 | GAAGAAAAAGGACGAGCAA |
| 2801 | AAGAAAAAGGACGAGCAAG |
| 2802 | AGAAAAAGGACGAGCAAGG |
| 2803 | GAAAAAGGACGAGCAAGGA |
| 2804 | AAAAAGGACGAGCAAGGAA |
| 2805 | AAAAGGACGAGCAAGGAAA |
| 2806 | AAAGGACGAGCAAGGAAAG |
| 2807 | AAGGACGAGCAAGGAAAGC |
| 2808 | AGGACGAGCAAGGAAAGCA |
| 2809 | GGACGAGCAAGGAAAGCAA |
| 2810 | GACGAGCAAGGAAAGCAAG |
| 2811 | ACGAGCAAGGAAAGCAAGA |
| 2812 | CGAGCAAGGAAAGCAAGAU |
| 2813 | GAGCAAGGAAAGCAAGAUA |
| 2814 | AGCAAGGAAAGCAAGAUAA |
| 2815 | GCAAGGAAAGCAAGAUAAU |
| 2816 | CAAGGAAAGCAAGAUAAUA |
| 2817 | AAGGAAAGCAAGAUAAUAC |
| 2818 | AGGAAAGCAAGAUAAUACA |
| 2819 | GGAAAGCAAGAUAAUACAA |
| 2820 | GAAAGCAAGAUAAUACAAA |
| 2821 | AAAGCAAGAUAAUACAAAC |
| 2822 | AAGCAAGAUAAUACAAACG |
| 2823 | AGCAAGAUAAUACAAACGG |
| 2824 | GCAAGAUAAUACAAACGGG |
| 2825 | CAAGAUAAUACAAACGGGA |
| 2826 | AAGAUAAUACAAACGGGAA |
| 2827 | AGAUAAUACAAACGGGAAC |
| 2828 | GAUAAUACAAACGGGAACA |
| 2829 | AUAAUACAAACGGGAACAG |
| 2830 | UAAUACAAACGGGAACAGA |
| 2831 | AAUACAAACGGGAACAGAA |
| 2832 | AUACAAACGGGAACAGAAA |
| 2833 | UACAAACGGGAACAGAAAU |
| 2834 | ACAAACGGGAACAGAAAUC |
| 2835 | CAAACGGGAACAGAAAUCG |
| 2836 | AAACGGGAACAGAAAUCGG |
| 2837 | AACGGGAACAGAAAUCGGA |
| 2838 | ACGGGAACAGAAAUCGGAA |
| 2839 | CGGGAACAGAAAUCGGAAA |
| 2840 | GGGAACAGAAAUCGGAAAG |
| 2841 | GGAACAGAAAUCGGAAAGA |
| 2842 | GAACAGAAAUCGGAAAGAC |
| 2843 | AACAGAAAUCGGAAAGACA |
| 2844 | ACAGAAAUCGGAAAGACAC |
| 2845 | CAGAAAUCGGAAAGACACC |
| 2846 | AGAAAUCGGAAAGACACCA |
| 2847 | GAAAUCGGAAAGACACCAA |
| 2848 | AAAUCGGAAAGACACCAAA |
| 2849 | AAUCGGAAAGACACCAAAG |
| 2850 | AUCGGAAAGACACCAAAGA |
| 2851 | UCGGAAAGACACCAAAGAU |
| 2852 | CGGAAAGACACCAAAGAUG |
| 2853 | GGAAAGACACCAAAGAUGC |
| 2854 | GAAAGACACCAAAGAUGCA |
| 2855 | AAAGACACCAAAGAUGCAA |
| 2856 | AAGACACCAAAGAUGCAAA |
| 2857 | AGACACCAAAGAUGCAAAG |
| 2858 | GACACCAAAGAUGCAAAGU |
| 2859 | ACACCAAAGAUGCAAAGUC |
| 2860 | CACCAAAGAUGCAAAGUCU |
| 2861 | ACCAAAGAUGCAAAGUCUG |
| 2862 | CCAAAGAUGCAAAGUCUGG |
| 2863 | CAAAGAUGCAAAGUCUGGC |
| 2864 | AAAGAUGCAAAGUCUGGCA |
| 2865 | AAGAUGCAAAGUCUGGCAC |
| 2866 | AGAUGCAAAGUCUGGCACC |
| 2867 | GAUGCAAAGUCUGGCACCA |
| 2868 | AUGCAAAGUCUGGCACCAA |
| 2869 | UGCAAAGUCUGGCACCAAG |
| 2870 | GCAAAGUCUGGCACCAAGA |
| 2871 | CAAAGUCUGGCACCAAGAA |
| 2872 | AAAGUCUGGCACCAAGAAG |
| 2873 | AAGUCUGGCACCAAGAAGA |
| 2874 | AGUCUGGCACCAAGAAGAG |
| 2875 | GUCUGGCACCAAGAAGAGG |
| 2876 | UCUGGCACCAAGAAGAGGA |
| 2877 | CUGGCACCAAGAAGAGGAA |
| 2878 | UGGCACCAAGAAGAGGAAG |
| 2879 | GGCACCAAGAAGAGGAAGA |
| 2880 | GCACCAAGAAGAGGAAGAG |
| 2881 | CACCAAGAAGAGGAAGAGC |
| 2882 | ACCAAGAAGAGGAAGAGCA |
| 2883 | CCAAGAAGAGGAAGAGCAA |
| 2884 | CAAGAAGAGGAAGAGCAAA |
| 2885 | AAGAAGAGGAAGAGCAAAC |
| 2886 | AGAAGAGGAAGAGCAAACA |
| 2887 | GAAGAGGAAGAGCAAACAG |
| 2888 | AAGAGGAAGAGCAAACAGA |
| 2889 | AGAGGAAGAGCAAACAGAG |
| 2890 | GAGGAAGAGCAAACAGAGG |
| 2891 | AGGAAGAGCAAACAGAGGG |
| 2892 | GGAAGAGCAAACAGAGGGG |
| 2893 | GAAGAGCAAACAGAGGGGG |
| 2894 | AAGAGCAAACAGAGGGGGG |
| 2895 | AGAGCAAACAGAGGGGGGC |
| 2896 | GAGCAAACAGAGGGGGGCU |
| 2897 | AGCAAACAGAGGGGGGCUG |
| 2898 | GCAAACAGAGGGGGGCUGU |
| 2899 | CAAACAGAGGGGGGCUGUG |
| 2900 | AAACAGAGGGGGGCUGUGG |
| 2901 | AACAGAGGGGGGCUGUGGC |
| 2902 | ACAGAGGGGGGCUGUGGCC |
| 2903 | CAGAGGGGGGCUGUGGCCC |
| 2904 | AGAGGGGGGCUGUGGCCCC |
| 2905 | GAGGGGGGCUGUGGCCCCC |
| 2906 | AGGGGGGCUGUGGCCCCCA |
| 2907 | GGGGGGCUGUGGCCCCCAC |
| 2908 | GGGGGCUGUGGCCCCCACC |
| 2909 | GGGGCUGUGGCCCCCACCA |
| 2910 | GGGCUGUGGCCCCCACCAC |
| 2911 | GGCUGUGGCCCCCACCACA |
| 2912 | GCUGUGGCCCCCACCACAU |
| 2913 | CUGUGGCCCCCACCACAUC |
| 2914 | UGUGGCCCCCACCACAUCC |
| 2915 | GUGGCCCCCACCACAUCCG |
| 2916 | UGGCCCCCACCACAUCCGC |
| 2917 | GGCCCCCACCACAUCCGCC |
| 2918 | GCCCCCACCACAUCCGCCA |
| 2919 | CCCCCACCACAUCCGCCAG |
| 2920 | CCCCACCACAUCCGCCAGC |
| 2921 | CCCACCACAUCCGCCAGCC |
| 2922 | CCACCACAUCCGCCAGCCC |
| 2923 | CACCACAUCCGCCAGCCCU |
| 2924 | ACCACAUCCGCCAGCCCUG |
| 2925 | CCACAUCCGCCAGCCCUGC |
| 2926 | CACAUCCGCCAGCCCUGCC |
| 2927 | ACAUCCGCCAGCCCUGCCC |
| 2928 | CAUCCGCCAGCCCUGCCCA |
| 2929 | AUCCGCCAGCCCUGCCCAA |
| 2930 | UCCGCCAGCCCUGCCCAAU |
| 2931 | CCGCCAGCCCUGCCCAAUA |
| 2932 | CGCCAGCCCUGCCCAAUAG |
| 2933 | GCCAGCCCUGCCCAAUAGC |
| 2934 | CCAGCCCUGCCCAAUAGCU |
| 2935 | CAGCCCUGCCCAAUAGCUG |
| 2936 | AGCCCUGCCCAAUAGCUGC |
| 2937 | GCCCUGCCCAAUAGCUGCC |
| 2938 | CCCUGCCCAAUAGCUGCCC |
| 2939 | CCUGCCCAAUAGCUGCCCC |
| 2940 | CUGCCCAAUAGCUGCCCCU |
| 2941 | UGCCCAAUAGCUGCCCCUU |
| 2942 | GCCCAAUAGCUGCCCCUUU |
| 2943 | CCCAAUAGCUGCCCCUUUA |
| 2944 | CCAAUAGCUGCCCCUUUAC |
| 2945 | CAAUAGCUGCCCCUUUACG |
| 2946 | AAUAGCUGCCCCUUUACGU |
| 2947 | AUAGCUGCCCCUUUACGUC |
| 2948 | UAGCUGCCCCUUUACGUCA |
| 2949 | AGCUGCCCCUUUACGUCAC |
| 2950 | GCUGCCCCUUUACGUCACC |
| 2951 | CUGCCCCUUUACGUCACCU |
| 2952 | UGCCCCUUUACGUCACCUG |
| 2953 | GCCCCUUUACGUCACCUGA |
| 2954 | CCCCUUUACGUCACCUGAC |
| 2955 | CCCUUUACGUCACCUGACG |
| 2956 | CCUUUACGUCACCUGACGG |
| 2957 | CUUUACGUCACCUGACGGC |
| 2958 | UUUACGUCACCUGACGGCA |
| 2959 | UUACGUCACCUGACGGCAA |
| 2960 | UACGUCACCUGACGGCAAG |
| 2961 | ACGUCACCUGACGGCAAGA |
| 2962 | CGUCACCUGACGGCAAGAC |
| 2963 | GUCACCUGACGGCAAGACU |
| 2964 | UCACCUGACGGCAAGACUU |
| 2965 | CACCUGACGGCAAGACUUC |
| 2966 | ACCUGACGGCAAGACUUCA |
| 2967 | CCUGACGGCAAGACUUCAU |
| 2968 | CUGACGGCAAGACUUCAUU |
| 2969 | UGACGGCAAGACUUCAUUG |
| 2970 | GACGGCAAGACUUCAUUGC |
| 2971 | ACGGCAAGACUUCAUUGCU |
| 2972 | CGGCAAGACUUCAUUGCUG |
| 2973 | GGCAAGACUUCAUUGCUGC |
| 2974 | GCAAGACUUCAUUGCUGCU |
| 2975 | CAAGACUUCAUUGCUGCUA |
| 2976 | AAGACUUCAUUGCUGCUAU |
| 2977 | AGACUUCAUUGCUGCUAUG |
| 2978 | GACUUCAUUGCUGCUAUGU |
| 2979 | ACUUCAUUGCUGCUAUGUA |
| 2980 | CUUCAUUGCUGCUAUGUAU |
| 2981 | UUCAUUGCUGCUAUGUAUA |
| 2982 | UCAUUGCUGCUAUGUAUAU |
| 2983 | CAUUGCUGCUAUGUAUAUG |
| 2984 | AUUGCUGCUAUGUAUAUGA |
| 2985 | UUGCUGCUAUGUAUAUGAA |
| 2986 | UGCUGCUAUGUAUAUGAAA |
| 2987 | GCUGCUAUGUAUAUGAAAG |
| 2988 | CUGCUAUGUAUAUGAAAGC |
| 2989 | UGCUAUGUAUAUGAAAGCU |
| 2990 | GCUAUGUAUAUGAAAGCUU |
| 2991 | CUAUGUAUAUGAAAGCUUU |
| 2992 | UAUGUAUAUGAAAGCUUUA |
| 2993 | AUGUAUAUGAAAGCUUUAU |
| 2994 | UGUAUAUGAAAGCUUUAUU |
| 2995 | GUAUAUGAAAGCUUUAUUG |
| 2996 | UAUAUGAAAGCUUUAUUGA |
| 2997 | AUAUGAAAGCUUUAUUGAA |
| 2998 | UAUGAAAGCUUUAUUGAAC |
| 2999 | AUGAAAGCUUUAUUGAACC |
| 3000 | UGAAAGCUUUAUUGAACCA |
| 3001 | GAAAGCUUUAUUGAACCAG |
| 3002 | AAAGCUUUAUUGAACCAGA |
| 3003 | AAGCUUUAUUGAACCAGAG |
| 3004 | AGCUUUAUUGAACCAGAGC |
| 3005 | GCUUUAUUGAACCAGAGCA |
| 3006 | CUUUAUUGAACCAGAGCAC |
| 3007 | UUUAUUGAACCAGAGCACU |
| 3008 | UUAUUGAACCAGAGCACUG |
| 3009 | UAUUGAACCAGAGCACUGC |
| 3010 | AUUGAACCAGAGCACUGCU |
| 3011 | UUGAACCAGAGCACUGCUA |
| 3012 | UGAACCAGAGCACUGCUAC |
| 3013 | GAACCAGAGCACUGCUACA |
| 3014 | AACCAGAGCACUGCUACAC |
| 3015 | ACCAGAGCACUGCUACACA |
| 3016 | CCAGAGCACUGCUACACAA |
| 3017 | CAGAGCACUGCUACACAAC |
| 3018 | AGAGCACUGCUACACAACA |
| 3019 | GAGCACUGCUACACAACAU |
| 3020 | AGCACUGCUACACAACAUU |
| 3021 | GCACUGCUACACAACAUUA |
| 3022 | CACUGCUACACAACAUUAC |
| 3023 | ACUGCUACACAACAUUACA |
| 3024 | CUGCUACACAACAUUACAC |
| 3025 | UGCUACACAACAUUACACA |
| 3026 | GCUACACAACAUUACACAU |
| 3027 | CUACACAACAUUACACAUG |
| 3028 | UACACAACAUUACACAUGU |
| 3029 | ACACAACAUUACACAUGUC |
| 3030 | CACAACAUUACACAUGUCA |
| 3031 | ACAACAUUACACAUGUCAG |
| 3032 | CAACAUUACACAUGUCAGA |
| 3033 | AACAUUACACAUGUCAGAA |
| 3034 | ACAUUACACAUGUCAGAAA |
| 3035 | CAUUACACAUGUCAGAAAG |
| 3036 | AUUACACAUGUCAGAAAGA |
| 3037 | UUACACAUGUCAGAAAGAC |
| 3038 | UACACAUGUCAGAAAGACA |
| 3039 | ACACAUGUCAGAAAGACAG |
| 3040 | CACAUGUCAGAAAGACAGA |
| 3041 | ACAUGUCAGAAAGACAGAG |
| 3042 | CAUGUCAGAAAGACAGAGC |
| 3043 | AUGUCAGAAAGACAGAGCU |
| 3044 | UGUCAGAAAGACAGAGCUA |
| 3045 | GUCAGAAAGACAGAGCUAU |
| 3046 | UCAGAAAGACAGAGCUAUA |
| 3047 | CAGAAAGACAGAGCUAUAC |
| 3048 | AGAAAGACAGAGCUAUACU |
| 3049 | GAAAGACAGAGCUAUACUC |
| 3050 | AAAGACAGAGCUAUACUCC |
| 3051 | AAGACAGAGCUAUACUCCU |
| 3052 | AGACAGAGCUAUACUCCUA |
| 3053 | GACAGAGCUAUACUCCUAG |
| 3054 | ACAGAGCUAUACUCCUAGA |
| 3055 | CAGAGCUAUACUCCUAGAC |
| 3056 | AGAGCUAUACUCCUAGACU |
| 3057 | GAGCUAUACUCCUAGACUC |
| 3058 | AGCUAUACUCCUAGACUCG |
| 3059 | GCUAUACUCCUAGACUCGA |
| 3060 | CUAUACUCCUAGACUCGAC |
| 3061 | UAUACUCCUAGACUCGACA |
| 3062 | AUACUCCUAGACUCGACAG |
| 3063 | UACUCCUAGACUCGACAGA |
| 3064 | ACUCCUAGACUCGACAGAA |
| 3065 | CUCCUAGACUCGACAGAAG |
| 3066 | UCCUAGACUCGACAGAAGC |
| 3067 | CCUAGACUCGACAGAAGCC |
| 3068 | CUAGACUCGACAGAAGCCA |
| 3069 | UAGACUCGACAGAAGCCAC |
| 3070 | AGACUCGACAGAAGCCACA |
| 3071 | GACUCGACAGAAGCCACAU |
| 3072 | ACUCGACAGAAGCCACAUC |
| 3073 | CUCGACAGAAGCCACAUCC |
| 3074 | UCGACAGAAGCCACAUCCA |
| 3075 | CGACAGAAGCCACAUCCAC |
| 3076 | GACAGAAGCCACAUCCACA |
| 3077 | ACAGAAGCCACAUCCACAA |
| 3078 | CAGAAGCCACAUCCACAAC |
| 3079 | AGAAGCCACAUCCACAACA |
| 3080 | GAAGCCACAUCCACAACAC |
| 3081 | AAGCCACAUCCACAACACU |
| 3082 | AGCCACAUCCACAACACUU |
| 3083 | GCCACAUCCACAACACUUA |
| 3084 | CCACAUCCACAACACUUAA |
| 3085 | CACAUCCACAACACUUAAG |
| 3086 | ACAUCCACAACACUUAAGG |
| 3087 | CAUCCACAACACUUAAGGA |
| 3088 | AUCCACAACACUUAAGGAG |
| 3089 | UCCACAACACUUAAGGAGG |
| 3090 | CCACAACACUUAAGGAGGC |
| 3091 | CACAACACUUAAGGAGGCG |
| 3092 | ACAACACUUAAGGAGGCGG |
| 3093 | CAACACUUAAGGAGGCGGU |
| 3094 | AACACUUAAGGAGGCGGUA |
| 3095 | ACACUUAAGGAGGCGGUAC |
| 3096 | CACUUAAGGAGGCGGUACC |
| 3097 | ACUUAAGGAGGCGGUACCC |
| 3098 | CUUAAGGAGGCGGUACCCC |
| 3099 | UUAAGGAGGCGGUACCCCC |
| 3100 | UAAGGAGGCGGUACCCCCG |
| 3101 | AAGGAGGCGGUACCCCCGG |
| 3102 | AGGAGGCGGUACCCCCGGC |
| 3103 | GGAGGCGGUACCCCCGGCA |
| 3104 | GAGGCGGUACCCCCGGCAC |
| 3105 | AGGCGGUACCCCCGGCACC |
| 3106 | GGCGGUACCCCCGGCACCA |
| 3107 | GCGGUACCCCCGGCACCAU |
| 3108 | CGGUACCCCCGGCACCAUG |
| 3109 | GGUACCCCCGGCACCAUGA |
| 3110 | GUACCCCCGGCACCAUGAA |
| 3111 | UACCCCCGGCACCAUGAAU |
| 3112 | ACCCCCGGCACCAUGAAUG |
| 3113 | CCCCCGGCACCAUGAAUGG |
| 3114 | CCCCGGCACCAUGAAUGGC |
| 3115 | CCCGGCACCAUGAAUGGCA |
| 3116 | CCGGCACCAUGAAUGGCAU |
| 3117 | CGGCACCAUGAAUGGCAUC |
| 3118 | GGCACCAUGAAUGGCAUCC |
| 3119 | GCACCAUGAAUGGCAUCCA |
| 3120 | CACCAUGAAUGGCAUCCAU |
| 3121 | ACCAUGAAUGGCAUCCAUU |
| 3122 | CCAUGAAUGGCAUCCAUUG |
| 3123 | CAUGAAUGGCAUCCAUUGG |
| 3124 | AUGAAUGGCAUCCAUUGGG |
| 3125 | UGAAUGGCAUCCAUUGGGG |
| 3126 | GAAUGGCAUCCAUUGGGGC |
| 3127 | AAUGGCAUCCAUUGGGGCA |
| 3128 | AUGGCAUCCAUUGGGGCAG |
| 3129 | UGGCAUCCAUUGGGGCAGU |
| 3130 | GGCAUCCAUUGGGGCAGUG |
| 3131 | GCAUCCAUUGGGGCAGUGG |
| 3132 | CAUCCAUUGGGGCAGUGGG |
| 3133 | AUCCAUUGGGGCAGUGGGA |
| 3134 | UCCAUUGGGGCAGUGGGAC |
| 3135 | CCAUUGGGGCAGUGGGACA |
| 3136 | CAUUGGGGCAGUGGGACAC |
| 3137 | AUUGGGGCAGUGGGACACU |
| 3138 | UUGGGGCAGUGGGACACUG |
| 3139 | UGGGGCAGUGGGACACUGC |
| 3140 | GGGGCAGUGGGACACUGCA |
| 3141 | GGGCAGUGGGACACUGCAG |
| 3142 | GGCAGUGGGACACUGCAGG |
| 3143 | GCAGUGGGACACUGCAGGA |
| 3144 | CAGUGGGACACUGCAGGAC |
| 3145 | AGUGGGACACUGCAGGACC |
| 3146 | GUGGGACACUGCAGGACCA |
| 3147 | UGGGACACUGCAGGACCAG |
| 3148 | GGGACACUGCAGGACCAGA |
| 3149 | GGACACUGCAGGACCAGAG |
| 3150 | GACACUGCAGGACCAGAGG |
| 3151 | ACACUGCAGGACCAGAGGU |
| 3152 | CACUGCAGGACCAGAGGUG |
| 3153 | ACUGCAGGACCAGAGGUGA |
| 3154 | CUGCAGGACCAGAGGUGAG |
| 3155 | UGCAGGACCAGAGGUGAGG |
| 3156 | GCAGGACCAGAGGUGAGGA |
| 3157 | CAGGACCAGAGGUGAGGAU |
| 3158 | AGGACCAGAGGUGAGGAUG |
| 3159 | GGACCAGAGGUGAGGAUGA |
| 3160 | GACCAGAGGUGAGGAUGAA |
| 3161 | ACCAGAGGUGAGGAUGAAC |
| 3162 | CCAGAGGUGAGGAUGAACC |
| 3163 | CAGAGGUGAGGAUGAACCA |
| 3164 | AGAGGUGAGGAUGAACCAA |
| 3165 | GAGGUGAGGAUGAACCAAG |
| 3166 | AGGUGAGGAUGAACCAAGG |
| 3167 | GGUGAGGAUGAACCAAGGA |
| 3168 | GUGAGGAUGAACCAAGGAU |
| 3169 | UGAGGAUGAACCAAGGAUG |
| 3170 | GAGGAUGAACCAAGGAUGG |
| 3171 | AGGAUGAACCAAGGAUGGG |
| 3172 | GGAUGAACCAAGGAUGGGG |
| 3173 | GAUGAACCAAGGAUGGGGG |
| 3174 | AUGAACCAAGGAUGGGGGC |
| 3175 | UGAACCAAGGAUGGGGGCA |
| 3176 | GAACCAAGGAUGGGGGCAU |
| 3177 | AACCAAGGAUGGGGGCAUG |
| 3178 | ACCAAGGAUGGGGGCAUGG |
| 3179 | CCAAGGAUGGGGGCAUGGG |
| 3180 | CAAGGAUGGGGGCAUGGGG |
| 3181 | AAGGAUGGGGGCAUGGGGC |
| 3182 | AGGAUGGGGGCAUGGGGCC |
| 3183 | GGAUGGGGGCAUGGGGCCU |
| 3184 | GAUGGGGGCAUGGGGCCUU |
| 3185 | AUGGGGGCAUGGGGCCUUG |
| 3186 | UGGGGGCAUGGGGCCUUGG |
| 3187 | GGGGGCAUGGGGCCUUGGG |
| 3188 | GGGGCAUGGGGCCUUGGGA |
| 3189 | GGGCAUGGGGCCUUGGGAC |
| 3190 | GGCAUGGGGCCUUGGGACA |
| 3191 | GCAUGGGGCCUUGGGACAC |
| 3192 | CAUGGGGCCUUGGGACACU |
| 3193 | AUGGGGCCUUGGGACACUU |
| 3194 | UGGGGCCUUGGGACACUUG |
| 3195 | GGGGCCUUGGGACACUUGC |
| 3196 | GGGCCUUGGGACACUUGCC |
| 3197 | GGCCUUGGGACACUUGCCU |
| 3198 | GCCUUGGGACACUUGCCUU |
| 3199 | CCUUGGGACACUUGCCUUG |
| 3200 | CUUGGGACACUUGCCUUGU |
| 3201 | UUGGGACACUUGCCUUGUG |
| 3202 | UGGGACACUUGCCUUGUGC |
| 3203 | GGGACACUUGCCUUGUGCC |
| 3204 | GGACACUUGCCUUGUGCCC |
| 3205 | GACACUUGCCUUGUGCCCA |
| 3206 | ACACUUGCCUUGUGCCCAG |
| 3207 | CACUUGCCUUGUGCCCAGC |
| 3208 | ACUUGCCUUGUGCCCAGCC |
| 3209 | CUUGCCUUGUGCCCAGCCA |
| 3210 | UUGCCUUGUGCCCAGCCAG |
| 3211 | UGCCUUGUGCCCAGCCAGC |
| 3212 | GCCUUGUGCCCAGCCAGCC |
| 3213 | CCUUGUGCCCAGCCAGCCA |
| 3214 | CUUGUGCCCAGCCAGCCAC |
| 3215 | UUGUGCCCAGCCAGCCACG |
| 3216 | UGUGCCCAGCCAGCCACGU |
| 3217 | GUGCCCAGCCAGCCACGUG |
| 3218 | UGCCCAGCCAGCCACGUGG |
| 3219 | GCCCAGCCAGCCACGUGGA |
| 3220 | CCCAGCCAGCCACGUGGAC |
| 3221 | CCAGCCAGCCACGUGGACU |
| 3222 | CAGCCAGCCACGUGGACUG |
| 3223 | AGCCAGCCACGUGGACUGG |
| 3224 | GCCAGCCACGUGGACUGGA |
| 3225 | CCAGCCACGUGGACUGGAU |
| 3226 | CAGCCACGUGGACUGGAUU |
| 3227 | AGCCACGUGGACUGGAUUU |
| 3228 | GCCACGUGGACUGGAUUUC |
| 3229 | CCACGUGGACUGGAUUUCU |
| 3230 | CACGUGGACUGGAUUUCUG |
| 3231 | ACGUGGACUGGAUUUCUGC |
| 3232 | CGUGGACUGGAUUUCUGCU |
| 3233 | GUGGACUGGAUUUCUGCUC |
| 3234 | UGGACUGGAUUUCUGCUCU |
| 3235 | GGACUGGAUUUCUGCUCUU |
| 3236 | GACUGGAUUUCUGCUCUUC |
| 3237 | ACUGGAUUUCUGCUCUUCC |
| 3238 | CUGGAUUUCUGCUCUUCCA |
| 3239 | UGGAUUUCUGCUCUUCCAG |
| 3240 | GGAUUUCUGCUCUUCCAGA |
| 3241 | GAUUUCUGCUCUUCCAGAC |
| 3242 | AUUUCUGCUCUUCCAGACC |
| 3243 | UUUCUGCUCUUCCAGACCG |
| 3244 | UUCUGCUCUUCCAGACCGG |
| 3245 | UCUGCUCUUCCAGACCGGG |
| 3246 | CUGCUCUUCCAGACCGGGG |
| 3247 | UGCUCUUCCAGACCGGGGA |
| 3248 | GCUCUUCCAGACCGGGGAA |
| 3249 | CUCUUCCAGACCGGGGAAC |
| 3250 | UCUUCCAGACCGGGGAACU |
| 3251 | CUUCCAGACCGGGGAACUG |
| 3252 | UUCCAGACCGGGGAACUGG |
| 3253 | UCCAGACCGGGGAACUGGA |
| 3254 | CCAGACCGGGGAACUGGAC |
| 3255 | CAGACCGGGGAACUGGACU |
| 3256 | AGACCGGGGAACUGGACUC |
| 3257 | GACCGGGGAACUGGACUCA |
| 3258 | ACCGGGGAACUGGACUCAC |
| 3259 | CCGGGGAACUGGACUCACA |
| 3260 | CGGGGAACUGGACUCACAU |
| 3261 | GGGGAACUGGACUCACAUA |
| 3262 | GGGAACUGGACUCACAUAA |
| 3263 | GGAACUGGACUCACAUAAA |
| 3264 | GAACUGGACUCACAUAAAG |
| 3265 | AACUGGACUCACAUAAAGG |
| 3266 | ACUGGACUCACAUAAAGGC |
| 3267 | CUGGACUCACAUAAAGGCA |
| 3268 | UGGACUCACAUAAAGGCAA |
| 3269 | GGACUCACAUAAAGGCAAU |
| 3270 | GACUCACAUAAAGGCAAUG |
| 3271 | ACUCACAUAAAGGCAAUGU |
| 3272 | CUCACAUAAAGGCAAUGUC |
| 3273 | UCACAUAAAGGCAAUGUCC |
| 3274 | CACAUAAAGGCAAUGUCCU |
| 3275 | ACAUAAAGGCAAUGUCCUC |
| 3276 | CAUAAAGGCAAUGUCCUCU |
| 3277 | AUAAAGGCAAUGUCCUCUU |
| 3278 | UAAAGGCAAUGUCCUCUUU |
| 3279 | AAAGGCAAUGUCCUCUUUC |
| 3280 | AAGGCAAUGUCCUCUUUCU |
| 3281 | AGGCAAUGUCCUCUUUCUC |
| 3282 | GGCAAUGUCCUCUUUCUCU |
| 3283 | GCAAUGUCCUCUUUCUCUU |
| 3284 | CAAUGUCCUCUUUCUCUUC |
| 3285 | AAUGUCCUCUUUCUCUUCC |
| 3286 | AUGUCCUCUUUCUCUUCCC |
| 3287 | UGUCCUCUUUCUCUUCCCC |
| 3288 | GUCCUCUUUCUCUUCCCCC |
| 3289 | UCCUCUUUCUCUUCCCCCC |
| 3290 | CCUCUUUCUCUUCCCCCCA |
| 3291 | CUCUUUCUCUUCCCCCCAA |
| 3292 | UCUUUCUCUUCCCCCCAAC |
| 3293 | CUUUCUCUUCCCCCCAACC |
| 3294 | UUUCUCUUCCCCCCAACCC |
| 3295 | UUCUCUUCCCCCCAACCCU |
| 3296 | UCUCUUCCCCCCAACCCUU |
| 3297 | CUCUUCCCCCCAACCCUUU |
| 3298 | UCUUCCCCCCAACCCUUUA |
| 3299 | CUUCCCCCCAACCCUUUAU |
| 3300 | UUCCCCCCAACCCUUUAUU |
| 3301 | UCCCCCCAACCCUUUAUUU |
| 3302 | CCCCCCAACCCUUUAUUUU |
| 3303 | CCCCCAACCCUUUAUUUUG |
| 3304 | CCCCAACCCUUUAUUUUGU |
| 3305 | CCCAACCCUUUAUUUUGUG |
| 3306 | CCAACCCUUUAUUUUGUGU |
| 3307 | CAACCCUUUAUUUUGUGUU |
| 3308 | AACCCUUUAUUUUGUGUUU |
| 3309 | ACCCUUUAUUUUGUGUUUU |
| 3310 | CCCUUUAUUUUGUGUUUUA |
| 3311 | CCUUUAUUUUGUGUUUUAA |
| 3312 | CUUUAUUUUGUGUUUUAAG |
| 3313 | UUUAUUUUGUGUUUUAAGC |
| 3314 | UUAUUUUGUGUUUUAAGCU |
| 3315 | UAUUUUGUGUUUUAAGCUG |
| 3316 | AUUUUGUGUUUUAAGCUGU |
| 3317 | UUUUGUGUUUUAAGCUGUA |
| 3318 | UUUGUGUUUUAAGCUGUAU |
| 3319 | UUGUGUUUUAAGCUGUAUG |
| 3320 | UGUGUUUUAAGCUGUAUGA |
| 3321 | GUGUUUUAAGCUGUAUGAC |
| 3322 | UGUUUUAAGCUGUAUGACU |
| 3323 | GUUUUAAGCUGUAUGACUU |
| 3324 | UUUUAAGCUGUAUGACUUU |
| 3325 | UUUAAGCUGUAUGACUUUA |
| 3326 | UUAAGCUGUAUGACUUUAU |
| 3327 | UAAGCUGUAUGACUUUAUC |
| 3328 | AAGCUGUAUGACUUUAUCA |
| 3329 | AGCUGUAUGACUUUAUCAC |
| 3330 | GCUGUAUGACUUUAUCACU |
| 3331 | CUGUAUGACUUUAUCACUG |
| 3332 | UGUAUGACUUUAUCACUGA |
| 3333 | GUAUGACUUUAUCACUGAG |
| 3334 | UAUGACUUUAUCACUGAGA |
| 3335 | AUGACUUUAUCACUGAGAA |
| 3336 | UGACUUUAUCACUGAGAAU |
| 3337 | GACUUUAUCACUGAGAAUA |
| 3338 | ACUUUAUCACUGAGAAUAA |
| 3339 | CUUUAUCACUGAGAAUAAU |
| 3340 | UUUAUCACUGAGAAUAAUA |
| 3341 | UUAUCACUGAGAAUAAUAC |
| 3342 | UAUCACUGAGAAUAAUACA |
| 3343 | AUCACUGAGAAUAAUACAU |
| 3344 | UCACUGAGAAUAAUACAUG |
| 3345 | CACUGAGAAUAAUACAUGU |
| 3346 | ACUGAGAAUAAUACAUGUU |
| 3347 | CUGAGAAUAAUACAUGUUA |
| 3348 | UGAGAAUAAUACAUGUUAA |
| 3349 | GAGAAUAAUACAUGUUAAA |
| 3350 | AGAAUAAUACAUGUUAAAC |
| 3351 | GAAUAAUACAUGUUAAACG |
| 3352 | AAUAAUACAUGUUAAACGU |
| 3353 | AUAAUACAUGUUAAACGUU |
| 3354 | UAAUACAUGUUAAACGUUU |
| 3355 | AAUACAUGUUAAACGUUUG |
| 3356 | AUACAUGUUAAACGUUUGU |
| 3357 | UACAUGUUAAACGUUUGUG |
| 3358 | ACAUGUUAAACGUUUGUGG |
| 3359 | CAUGUUAAACGUUUGUGGU |
| 3360 | AUGUUAAACGUUUGUGGUA |
| 3361 | UGUUAAACGUUUGUGGUAA |
| 3362 | GUUAAACGUUUGUGGUAAG |
| 3363 | UUAAACGUUUGUGGUAAGA |
| 3364 | UAAACGUUUGUGGUAAGAG |
| 3365 | AAACGUUUGUGGUAAGAGG |
| 3366 | AACGUUUGUGGUAAGAGGU |
| 3367 | ACGUUUGUGGUAAGAGGUC |
| 3368 | CGUUUGUGGUAAGAGGUCA |
| 3369 | GUUUGUGGUAAGAGGUCAG |
| 3370 | UUUGUGGUAAGAGGUCAGU |
| 3371 | UUGUGGUAAGAGGUCAGUG |
| 3372 | UGUGGUAAGAGGUCAGUGG |
| 3373 | GUGGUAAGAGGUCAGUGGU |
| 3374 | UGGUAAGAGGUCAGUGGUA |
| 3375 | GGUAAGAGGUCAGUGGUAU |
| 3376 | GUAAGAGGUCAGUGGUAUC |
| 3377 | UAAGAGGUCAGUGGUAUCU |
| 3378 | AAGAGGUCAGUGGUAUCUG |
| 3379 | AGAGGUCAGUGGUAUCUGC |
| 3380 | GAGGUCAGUGGUAUCUGCC |
| 3381 | AGGUCAGUGGUAUCUGCCC |
| 3382 | GGUCAGUGGUAUCUGCCCU |
| 3383 | GUCAGUGGUAUCUGCCCUG |
| 3384 | UCAGUGGUAUCUGCCCUGA |
| 3385 | CAGUGGUAUCUGCCCUGAA |
| 3386 | AGUGGUAUCUGCCCUGAAU |
| 3387 | GUGGUAUCUGCCCUGAAUC |
| 3388 | UGGUAUCUGCCCUGAAUCU |
| 3389 | GGUAUCUGCCCUGAAUCUG |
| 3390 | GUAUCUGCCCUGAAUCUGC |
| 3391 | UAUCUGCCCUGAAUCUGCU |
| 3392 | AUCUGCCCUGAAUCUGCUU |
| 3393 | UCUGCCCUGAAUCUGCUUC |
| 3394 | CUGCCCUGAAUCUGCUUCA |
| 3395 | UGCCCUGAAUCUGCUUCAA |
| 3396 | GCCCUGAAUCUGCUUCAAA |
| 3397 | CCCUGAAUCUGCUUCAAAG |
| 3398 | CCUGAAUCUGCUUCAAAGA |
| 3399 | CUGAAUCUGCUUCAAAGAG |
| 3400 | UGAAUCUGCUUCAAAGAGU |
| 3401 | GAAUCUGCUUCAAAGAGUU |
| 3402 | AAUCUGCUUCAAAGAGUUA |
| 3403 | AUCUGCUUCAAAGAGUUAU |
| 3404 | UCUGCUUCAAAGAGUUAUU |
| 3405 | CUGCUUCAAAGAGUUAUUU |
| 3406 | UGCUUCAAAGAGUUAUUUC |
| 3407 | GCUUCAAAGAGUUAUUUCA |
| 3408 | CUUCAAAGAGUUAUUUCAA |
| 3409 | UUCAAAGAGUUAUUUCAAA |
| 3410 | UCAAAGAGUUAUUUCAAAU |
| 3411 | CAAAGAGUUAUUUCAAAUU |
| 3412 | AAAGAGUUAUUUCAAAUUA |
| 3413 | AAGAGUUAUUUCAAAUUAA |
| 3414 | AGAGUUAUUUCAAAUUAAA |
| 3415 | GAGUUAUUUCAAAUUAAAA |
| 3416 | AGUUAUUUCAAAUUAAAAG |
| 3417 | GUUAUUUCAAAUUAAAAGC |
| 3418 | UUAUUUCAAAUUAAAAGCA |
| 3419 | UAUUUCAAAUUAAAAGCAA |
| 3420 | AUUUCAAAUUAAAAGCAAA |
| 3421 | UUUCAAAUUAAAAGCAAAA |
| 3422 | UUCAAAUUAAAAGCAAAAC |
| 3423 | UCAAAUUAAAAGCAAAACA |
| 3424 | CAAAUUAAAAGCAAAACAA |
| 3425 | AAAUUAAAAGCAAAACAAA |
| 3426 | AAUUAAAAGCAAAACAAAA |
| 3427 | AUUAAAAGCAAAACAAAAC |
| 3428 | UUAAAAGCAAAACAAAACA |
| 3429 | UAAAAGCAAAACAAAACAA |
| 3430 | AAAAGCAAAACAAAACAAA |

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 100 nucleotides in length, more preferably about 19 to about 50 nucleotides in length, and even more preferably about 19 to about 23 nucleotides in length.

The degree of identity of a double-stranded region of a nucleic acid molecule to the targeted transcript should be at least 90% and more preferably 95-100%. The % identity of a nucleic acid molecule is determined by GAP (Needleman and Wunsch, 1970) analysis (GCG program) with a gap creation penalty=5, and a gap extension penalty=0.3. Preferably, the two sequences are aligned over their entire length.

The nucleic acid molecule may of course comprise sequences unrelated to the target which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example, the siRNA can be a nucleic acid molecule comprising self-complementary sense and antisense regions, wherein the antisense region comprises a nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, and others. In addition, as used herein, the term RNAi is equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression.

Preferred siRNA molecules comprise a nucleotide sequence that is identical to about 19 to 23 contiguous nucleotides of the target mRNA. In an embodiment, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the avian (preferably chickens) in which it is to be introduced, e.g., as determined by standard BLAST search.

By “shRNA” or “short-hairpin RNA” is meant an siRNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to 15 nucleotides which forms a single-stranded loop above the stem structure created by the two regions of base complementarity. Examples of sequences of a single-stranded loops are 5′ UUCAAGAGA 3′ and 5′ UUUGUGUAG 3′.

Included shRNAs are dual or bi-finger and multi-finger hairpin dsRNAs, in which the RNA molecule comprises two or more of such stem-loop structures separated by single-stranded spacer regions.

siRNAs can be generated in vitro by using a recombinant enzyme, such as T7 RNA polymerase, and DNA oligonucleotide templates, or can be prepared in vivo, for example, in cultured cells. In a preferred embodiment, the nucleic acid molecule is produced synthetically.

Strategies have been described for producing a hairpin siRNA from vectors containing, for example, a RNA polymerase III promoter. Various vectors have been constructed for generating hairpin siRNAs in host cells using either an H1-RNA or an snU6 RNA promoter (see SEQ ID NO's 7 to 9). A RNA molecule as described above (e.g., a first portion, a linking sequence, and a second portion) can be operably linked to such a promoter. When transcribed by RNA polymerase III, the first and second portions form a duplexed stem of a hairpin and the linking sequence forms a loop. The pSuper vector (OligoEngines Ltd., Seattle, Wash.) can also be used to generate siRNA.

Modifications or analogs of nucleotides can be introduced to improve the properties of the nucleic acid molecules of the invention. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

### Vectors and Host Cells

The present invention also provides a vector encoding a nucleic acid molecule comprising a double-stranded region, or single strand thereof, of the present invention. Preferably, the vector is an expression vector capable of expressing the open reading frame(s) encoding a dsRNA in a host cell and/or cell free system. The host cell can be any cell type such as, not limited to, bacterial, fungal, plant or animal cells, preferably an avian cell.

Typically, a vector of the invention comprises a promoter operably linked to an open reading frame encoding a nucleic acid molecule of the invention, or a strand thereof.

As used herein, the term “promoter” refers to a nucleic acid sequence which is able to direct transcription of an operably linked nucleic acid molecule and includes, for example, RNA polymerase II and RNA polymerase III promoters. Also included in this definition are those transcriptional regulatory elements (e.g., enhancers) that are sufficient to render promoter-dependent gene expression controllable in a cell type-specific, tissue-specific, or temporal-specific manner, or that are inducible by external agents or signals.

“Operably linked” as used herein refers to a functional relationship between two or more nucleic acid (e.g., DNA) segments. Typically, it refers to the functional relationship of a transcriptional regulatory element to a transcribed sequence. For example, a promoter is operably linked to a coding sequence, such as an open reading frame encoding a double-stranded RNA molecule defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

By “RNA polymerase III promoter” or “RNA pol III promoter” or “polymerase III promoter” or “pol III promoter” is meant any invertebrate, vertebrate, or mammalian promoter, e.g., chicken, human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. By U6 promoter (e.g., chicken U6, human U6, murine U6), H1 promoter, or 7SK promoter is meant any invertebrate, vertebrate, or mammalian promoter or polymorphic variant or mutant found in nature to interact with RNA polymerase III to transcribe its cognate RNA product, i.e., U6 RNA, H1 RNA, or 7SK RNA, respectively. Examples of suitable promoters include cU6-1 (SEQ ID NO:7), cU6-3 (SEQ ID NO:8), cU6-4 (SEQ ID NO:9) and c7SK (SEQ ID NO:10).

When E. coli is used as a host cell, there is no limitation other than that the vector should have an “ori” to amplify and mass-produce the vector in E. coli (e.g., JM109. DH5α, HB101, or XL1Blue), and a marker gene for selecting the transformed E. coli (e.g., a drug-resistance gene selected by a drug such as ampicillin, tetracycline, kanamycin, or chloramphenicol). For example, M13-series vectors, pUC-series vectors, pBR322, pBluescript, pCR-Script, and such can be used. pGEM-T, pDIRECT, pT7, and so on can also be used for subcloning and excision of the gene encoding the dsRNA as well as the vectors described above.

With regard to expression vectors for use in E. coli, such vectors include JM109, DH5α, HB101, or XL1 Blue, the vector should have a promoter such as lacZ promoter, araB promoter, or T7 promoter that can efficiently promote the expression of the desired gene in E. coli. Other examples of the vectors are “QIAexpress system” (Qiagen), pEGFP, and pET (for this vector, BL21, a strain expressing T7 RNA polymerase, is preferably used as the host).

In addition to the vectors for E. coli, for example, the vector may be a mammal-derived expression vector (e.g., pcDNA3 (Invitrogen), pEGF-BOS, pEF, and pCDM8), an insect cell-derived expression vector (e.g., “Bac-to-BAC baculovairus expression system” (GibcoBRL) and pBacPAK8), a plant-derived expression vector (e.g., pMH1 and pMH2), an animal virus-derived expression vector (e.g., pHSV, pMV, and pAdexLcw), a retrovirus-derived expression vector (e.g., pZIPneo), a yeast-derived expression vector (e.g., “Pichia Expression Kit” (Invitrogen), pNV11, and SP-Q01), or a Bacillus subtilis-derived expression vector (e.g., pPL608 and pKTH50).

In order to express nucleic acid molecules in animal cells, such as CHO, COS, Vero and NIH3T3 cells, the vector should have a promoter necessary for expression in such cells, e.g., SV40 promoter, MMLV-LTR promoter, EF1α promoter, CMV promoter, etc., and more preferably it has a marker gene for selecting transformants (for example, a drug resistance gene selected by a drug (e.g., neomycin, G418, etc.). Examples of vectors with these characteristics include pMAM, pDR2, pBK-RSV, pBK-CMV, pOPRSV and pOPI3.

Nucleic acid molecules comprising a double-stranded region of the present invention can be expressed in animals such as avians by, for example, inserting an open reading frame(s) encoding the nucleic acid into an appropriate vector and introducing the vector by the retrovirus method, liposome method, cationic liposome method, adenovirus method, and so on. The vectors used include, but are not limited to, adenoviral vectors (e.g., pAdexlcw) and retroviral vectors (e.g., pZIPneo). General techniques for gene manipulation, such as insertion of nucleic acids of the invention into a vector, can be performed according to conventional methods.

The present invention also provides a host cell into which an exogenous nucleic acid molecule, typically in a vector of the present invention, has been introduced. The host cell of this invention can be used as, for example, a production system for producing or expressing the nucleic acid molecule. For in vitro production, eukaryotic cells or prokaryotic cells can be used.

Useful eukaryotic host cells may be animal, plant, or fungi cells. As animal cells, mammalian cells such as CHO, COS, 3T3, myeloma, baby hamster kidney (BHK), HeLa, or Vero cells MDCK cells, DF1 cells, amphibian cells such as Xenopus oocytes, or insect cells such as Sf9, Sf21, or Tn5 cells can be used. CHO cells lacking DHFR gene (dhfr-CHO) or CHO K-1 may also be used. The vector can be introduced into the host cell by, for example, the calcium phosphate method, the DEAE-dextran method, cationic liposome DOTAP (Boehringer Mannheim) method, electroporation, lipofection, etc.

Useful prokaryotic cells include bacterial cells, such as E. coli, for example, JM109, DH5α, and HB101, or Bacillus subtilis.

Culture medium such as DMEM, MEM, RPMI-1640, or IMDM may be used for animal cells. The culture medium can be used with or without serum supplement such as fetal calf serum (FCS). The pH of the culture medium is preferably between about 6 and 8. Cells are typically cultured at about 30 to 40° C. for about 15 to 200 hr, and the culture medium may be replaced, aerated, or stirred if necessary.

### Compositions

The present invention also provides compositions comprising a nucleic acid molecule comprising a double-stranded region that can be administered to an avian egg. A composition comprising a nucleic acid molecule comprising a double-stranded region may contain a pharmaceutically acceptable carrier to render the composition suitable for administration.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, antibacterial agents, antifungal agents, gum arabic, acacia gum, sodium and calcium salts of phosphoric and sulfuric acids, polyvinylpyrrolidone and/or polyvinyl alcohol, saline, and water. In an embodiment, the carrier, excipient and/or diluent is phosphate buffered saline or water.

In an embodiment, the composition may also comprise a transfection promoting agent. Transfection promoting agents used to facilitate the uptake of nucleic acids into a living cell are well known within the art. Reagents enhancing transfection include chemical families of the types; polycations, dendrimers, DEAE Dextran, block copolymers and cationic lipids. Preferably, the transfection-promoting agent is a lipid-containing compound (or formulation), providing a positively charged hydrophilic region and a fatty acyl hydrophobic region enabling self-assembly in aqueous solution into vesicles generally known as micelles or liposomes, as well as lipopolyamines.

In another embodiment, the composition comprises a polymeric biomaterial such as chitosan.

It is understood that any conventional media or agent may be used so long as it is not incompatible with the compositions or methods of the invention.

### Administration

Administration of a nucleic acid molecule comprising a double-stranded region (including a composition comprising a nucleic acid molecule comprising a double-stranded region) is conveniently achieved by injection into the egg, and generally injection into the chorion allantoic fluid. Notwithstanding that the air sac is the preferred route of in ovo administration, other regions such as the yolk sac, air sac or amnionic cavity (amnion) may also be inoculated by injection. The hatchability rate might decrease slightly when the air sac is not the target for the administration although not necessarily at commercially unacceptable levels. The mechanism of injection is not critical to the practice of the present invention, although it is preferred that the needle does not cause undue damage to the egg or to the tissues and organs of the developing embryo or the extra-embryonic membranes surrounding the embryo.

Preferably, the nucleic acid molecule is administered within four days of the egg having been laid.

Generally, a hypodermic syringe fitted with an approximately 22 gauge needle is suitable. The method of the present invention is particularly well adapted for use with an automated injection system, such as those described in U.S. Pat. No. 4,903,635, U.S. Pat. No. 5,056,464, U.S. Pat. No. 5,136,979 and US 20060075973.

The nucleic acid molecule is administered in an effective amount sufficient to modify sex in at least some of the eggs which have been administered. The modification can be detected by comparing a suitable number of samples subjected to the method of the invention to a similar number that have not. Statistically significant variation in the sex of the birds between the two groups will be indicative that an effective amount has been administered. Other means of determining an effective amount for sex are well within the capacity of those skilled in the art.

Preferably, about 1 ng to 100 μg, more preferably about 100 ng to 1 μg, of nucleic acid is administered to the egg. Furthermore, it is preferred that the nucleic acid to be administered is in a volume of about 1 μl to 1 ml, more preferably about 10 μl to 500 μl.

### EXAMPLES

### Example 1

### Identification of shRNA Molecules for Down-Regulating DMRT1 Protein Production in Chickens

Selection of shRNA Sequences Targeting DMRT1

The present inventors identified 51 predicted shRNA sequences to target chicken Dmrt1 (Table 4).

There are several algorithms available to select potential siRNA sequences for specific target genes. Taxman et al. (2006) have specifically designed an algorithm to predict effective shRNA molecules and the present inventors have made their own modification to the algorithm to improve shRNA prediction. There are four criteria for shRNA selection using the Taxman algorithm. Three of the criteria are scored for out of a maximum number of 4 points. These criteria are: 1) C or G on the 5′ end of the sequence=1 point, A or T on 5′ end=−1 point; 2) A or T on the 3′ end=1 point, C or G on the 3′ end=−1 point; 3) 5 or more A or T in the seven 3′ bases=2 points, 4 A or T in the seven 3′ bases=1 point. shRNA sequences with the highest scores are preferred. The fourth criteria is based on a calculation for the free-energy of the 6 central bases of the shRNA sequence (bases 6-11 of the sense strand hybridised to bases 9-14 of the antisense strand). shRNAs with a central duplex ΔG>−12.9 kcal/mol are preferred.

The shRNA designer website uses this algorithm to provide a score for each shRNA target. Based on the algorithm and their calculated ΔG value, the present inventors chose 4 of the shRNA target finder shRNA sequences as potentially effective shRNAs to test for their ability to knockdown Dmrt1 gene expression. The selected sequences are shown in their 5′-3′ sequence in Table 5. These 4 sequences were used to construct ddRNAi plasmids for the expression of the 6 shRNAs.

Construction of ddRNAi Plasmids for Expression of Selected shRNAs

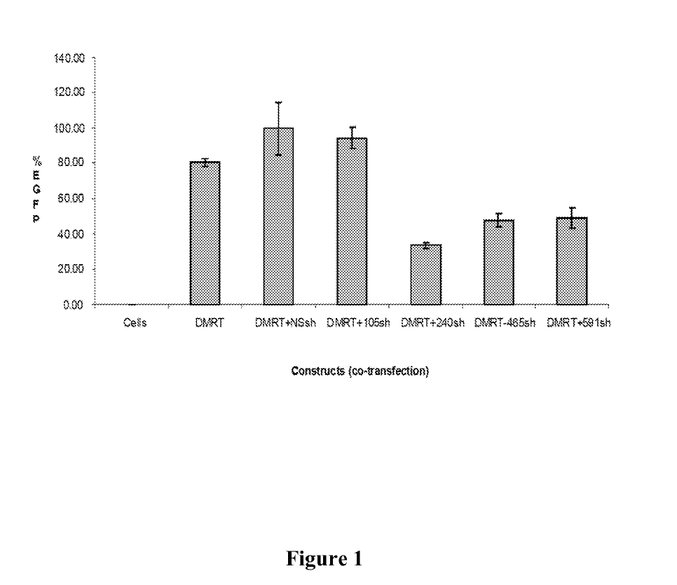

To construct the Dmrt1 shRNA expression constructs, two oligonucleotides complementary to each other were designed to contain the sense, loop, antisense and termination signal, followed by a spacer sequence (GGAA) and a BamHI restriction site for screening. In addition, the “bottom oligo (B)” or reverse oligonucleotide contained a SalI overhang at the 5′ end for insertion into the expression vector containing the chicken polymerase III promoter cU6-4 (DQ531570). Table 6 lists the shRNA targets to their corresponding oligonucleotides. The complementary oligonucleotides for each target shRNA, were annealed together and ligated into the PmeI-SalI digested pU6-4 vector. Full length clones were positive if linearised by BamHI digestion. All shRNA expression vectors were sequence confirmed. The four constructs were referred to as 105sh, 240sh, 465sh and 591sh as shown in FIG. 1. The non-silencing control shRNA construct (NSsh) was designed in the same way. However, the sequence used was that of an irrelevant target (ie. NP gene of influenza).

Each ddRNAi plasmid was constructed so that the start of each shRNA sequence was at the +1 position of the native U6 snRNA transcripts. All final shRNA expression vectors consisted of either one of the full length chicken U6 promoters, a shRNA sense sequence, a loop sequence, a shRNA antisense sequence, a termination sequence and a BamHI site. The loop sequence used in all shRNAs was 5′ UUCAAGAGA 3′.

TABLE 4
Algorithm selection of shRNA sequences targeting
Dmrt1.

| Target Sequence | Start Position | SEQ ID NO | Score |
|---|---|---|---|
| GGCACAAGCGGTTCTGCAT | 79 | 3435 | 3 |
| GACTGCCAGTGCAAGAAGT | 105 | 3436 | 3 |
| CTGAGCCAGTTGTCAAGAA | 238 | 3437 | 3 |
| GAGCCAGTTGTCAAGAAGA | 240 | 3438 | 3 |
| GACGGATGCTCATTCAGGA | 355 | 3439 | 3 |
| GCACGTCTGATTTGGTTGT | 409 | 3440 | 3 |
| GTGGACTCCACCTACTACA | 426 | 3441 | 3 |
| CCAGCCATCCCTGTATCCT | 455 | 3442 | 3 |
| CCATCCCTGTATCCTTACT | 459 | 3443 | 3 |
| CATCCCTGTATCCTTACTA | 460 | 3444 | 3 |
| CCCTGTATCCTTACTATAA | 463 | 3445 | 4 |
| CTGTATCCTTACTATAACA | 465 | 3446 | 4 |
| CTTACTATAACAACCTGTA | 472 | 3447 | 3 |
| CTCCCAGTACCAAATGGCA | 497 | 3448 | 3 |
| GCCACTGAGTCTTCCTCAA | 519 | 3449 | 3 |
| CTGAGTCTTCCTCAAGTGA | 523 | 3450 | 3 |
| GAGTCTTCCTCAAGTGAGA | 525 | 3451 | 3 |
| CTCCCAGCAACATACATGT | 591 | 3452 | 4 |
| CCCAGCAACATACATGTCA | 593 | 3453 | 3 |
| CCAGCAACATACATGTCAA | 594 | 3454 | 3 |
| CAGATGAAGGGAATGGAGA | 633 | 3455 | 3 |
| CCACCTGCGTCACACAGAT | 733 | 3456 | 3 |
| CACCTGCGTCACACAGATA | 734 | 3457 | 3 |
| CCTGCGTCACACAGATACT | 736 | 3458 | 3 |
| CTCCTACTCAGAGTCGAAA | 773 | 3459 | 3 |
| CACTGTTGCCTTGTTCTGT | 967 | 3460 | 3 |
| GGTGCCGTGATGTGTTTGT | 1189 | 3461 | 3 |
| GTGCCGTGATGTGTTTGTA | 1190 | 3462 | 4 |
| GCCGTGATGTGTTTGTAGT | 1192 | 3463 | 4 |
| CCTCGTATCGCCAAATTAA | 1239 | 3464 | 4 |
| GCCTCGACTTAGATTGCAA | 1283 | 3465 | 3 |
| CCTCGACTTAGATTGCAAT | 1284 | 3466 | 4 |
| CTCGACTTAGATTGCAATA | 1285 | 3467 | 4 |
| CGACTTAGATTGCAATATA | 1287 | 3468 | 4 |
| GACTTAGATTGCAATATAA | 1288 | 3469 | 4 |
| GCGGCCAGCAAACAAGTCT | 1307 | 3470 | 3 |
| GGCCAGCAAACAAGTCTCA | 1309 | 3471 | 3 |
| GCCAGCAAACAAGTCTCAA | 1310 | 3472 | 3 |
| CCAGCAAACAAGTCTCAAA | 1311 | 3473 | 3 |
| GCGTTTCTGCGAGTGTTAT | 1342 | 3474 | 4 |
| GTGTCCTCTTCCTGTGTTA | 1381 | 3475 | 3 |
| GTCCTCTTCCTGTGTTACA | 1383 | 3476 | 3 |
| CCTCTTCCTGTGTTACAGA | 1385 | 3477 | 3 |
| CTCTTCCTGTGTTACAGAA | 1386 | 3478 | 4 |
| GAAGCCAACCTGAAATGAA | 1402 | 3479 | 4 |
| GCCAACCTGAAATGAAACT | 1405 | 3480 | 4 |
| CCAACCTGAAATGAAACTA | 1406 | 3481 | 4 |
| CCTGAAATGAAACTAGTCT | 1410 | 3482 | 3 |
| GTTGCAGCTGTACCTGAAA | 1452 | 3483 | 3 |
| GCAGCTGTACCTGAAATAA | 1455 | 3484 | 4 |
| CAGCTGTACCTGAAATAAA | 1456 | 3485 | 4 |

TABLE 5
Sequence of Dmrt1 shRNAs.

| Target | | | | |
|---|---|---|---|---|
| Number | Position | Target Sequence | Score | ΔG |
| 1 | 105 bp | GACTGCCAGTGCAAGAAGT | 3 | −17.1 |
| | | (SEQ ID NO: 3436) | | |
| 2 | 240 bp | GAGCCAGTTGTCAAGAAGA | 3 | −12.7 |
| | | (SEQ ID NO: 3438) | | |
| 3 | 465 bp | CTGTATCCTTACTATAACA | 4 | −11.9 |
| | | (SEQ ID NO: 3446) | | |
| 4 | 591 bp | CTCCCAGCAACATACATGT | 4 | −14.0 |
| | | (SEQ ID NO: 3452) | | |

Testing Selected shRNAs for Knockdown of Dmrt1 Gene Expression

A reporter gene expression assay was used to test shRNAs for silencing of Dmrt1. The reporter gene was a transcriptional gene fusion of Dmrt1 inserted downstream of the 3′ end of the Enhanced Green Fluorescent Protein (EGFP) gene in pEGFP-C (Clontech). The reporter plasmid was constructed as follows: cDNA of Dmrt1 was reverse transcribed from total RNA isolated from 4 day old embryo's and cloned into the multiple cloning site of pCMV-Script (Stratagene). The Dmrt1 insert was removed from the cloning vector as a NotI-EcoRI fragment and cloned downstream of the EGFP gene in pEGFP-C (Clontech). The resulting plasmid was named pEGFP-Dmrt1. This plasmid was transfected into chicken DF-1 cells and expression of the transcriptional gene fusion was confirmed by measuring EGFP fluorescence using flow cytometry as described below.

Dmrt1 gene silencing assays were conducted by co-transfecting DF-1 cells with the pEGFP-Dmrt1 reporter plasmid and each of the ddRNAi plasmids expressing the Dmrt1 specific and control shRNAs. The co-transfection experiments were performed as follows: DF-1 cells (ATCC CRL-12203, chicken fibroblast) were maintained in a humidified atmosphere containing 5% CO₂ at 37° C. in Dulbecco's Modified Eagle's Medium (DMEM) containing 4.5 g/l glucose, 1.5 g/l sodium bicarbonate, 10% foetal calf serum (FCS), 2 mM L-glutamine supplemented with penicillin (100 Um') and streptomycin (100 μg/ml). DF1 cells were passaged as required using 0.25% (w/v) trypsin-ethylenediaminetetraacetic acid (EDTA).

TABLE 6
Sequence and details of primers used.

| Target | | | | |
|---|---|---|---|---|
| Number | Position | Target Sequence | Score | ΔG |
| 1 | 105 bp | GACTGCCAGTGCAAGAAGT | 3 | −17.1 |
| | | (SEQ ID NO: 3436) | | |

DMRT1-105T-U6.4
GACTGCCAGTGCAAGAAGTTTCAAGAGAACTTCTTGCACTGGCAGTCTTT
TTGGAAGGATCC (SEQ ID NO: 3486)
DMRT1-105B-U6.4
TCGAGGATCCTTCCAAAAAGACTGCCAGTGCAAGAAGTTCTCTTGAAAC
TTCTTGCACTGGCAGTC (SEQ ID NO: 3487)

| Target | | | | |
|---|---|---|---|---|
| Number | Position | Target Sequence | Score | ΔG |
| 2 | 240 bp | GAGCCAGTTGTCAAGAAGA | 3 | −12.7 |
| | | (SEQ ID NO: 3438) | | |

DMRT1-240T-U6.4
GAGCCAGTTGTCAAGAAGATTCAAGAGATCTTCTTGACAACTGGCTCTTT
TTGGAAGGATCC (SEQ ID NO: 3488)
DMRT1-240B-U6.4
TCGAGGATCCTTCCAAAAAGAGCCAGTTGTCAAGAAGATCTCTTGAATCT
TCTTGACAACTGGCTC (SEQ ID NO: 3489)

| Target | | | | |
|---|---|---|---|---|
| Number | Position | Target Sequence | Score | ΔG |
| 3 | 465 bp | CTGTATCCTTACTATAACA | 4 | −11.9 |
| | | (SEQ ID NO: 3446) | | |

DMRT1-465T-U6.4
CTGTATCCTTACTATAACATTCAAGAGATGTTATAGTAAGGATACAGTTT
TTGGAAGGATCC (SEQ ID NO: 3490)
DMRT1-465B-U6.4
TCGAGGATCCTTCCAAAAACTGTATCCTTACTATAACATCTCTTGAATGT
TATAGTAAGGATACAG (SEQ ID NO: 3491)

| Target | | | | |
|---|---|---|---|---|
| Number | Position | Target Sequence | Score | ΔG |
| 4 | 591 bp | CTCCCAGCAACATACATGT | 4 | −14.0 |
| | | (SEQ ID NO: 3452) | | |

DMRT1-591T-U6.4
CTCCCAGCAACATACATGTTTCAAGAGAACATGTATGTTGCTGGGAGTTT
TTGGAAGGATCC (SEQ ID NO: 3492)
DMRT1-591B-U6.4
TCGAGGATCCTTCCAAAAACTCCCAGCAACATACATGTTCTCTTGAAACA
TGTATGTTGCTGGGAG (SEQ ID NO: 3493)
Bold = spacer sequence;
Italics = BamHI restriction site;
Underline = SalI overhang

Co-transfection of pEGFP-Dmrt1 and ddRNAi plasmids for EGFP-Dmrt1 fusion silencing assays was conducted in DF-1 cells grown to 80-90% confluence, in 24 well culture plates (Nunc) for flow cytometry analysis. Cells were transfected with a total of 1 μg of plasmid DNA, per well, using Lipofectamine™2000 transfection reagent (Invitrogen). EGFP expression was analysed in transfected DF-1 cells at 60 hours post-transfection using flow cytometry analysis of transfections performed in triplicate. Cells were trypsinised using 100 μl of 0.25% trypsin-EDTA, pelleted at 2000 rpm for 5 minutes, washed once in 1 ml of cold phosphate buffered saline-A (PBSA) (Oxoid), twice in 1 ml of FACS-wash solution (PBSA+1% FCS) and resuspended in 250 μl of FACS-wash solution. Flow cytometry sampling was performed using a FACScalibur (Becton Dickinson) fluorescence activated cell sorter. Data acquisition and calculation of mean fluorescence intensity (MFI) values for triplicate co-transfection samples, was performed using CELLQuest software (Becton Dickinson). The results of the gene silencing assay are shown in FIG. 1. Compared to the negative control irrelevant shRNA expressed from NSsh, the Dmrt1 specific shRNAs were observed to knockdown expression of the reporter gene to varying levels. Dmrt1 shRNA 240sh induced the greatest level of gene silencing of approximately 60%.

### Example 2

### In Ovo Modulation of DMRT1 Gene Expression in Chickens

An siRNA targeted to a conserved exon of the chicken DMRT1 gene was designed using the Ambion siRNA Target Finder tool (www.ambion.com). The chosen siRNA was designated DMRT1-343-siRNA (5′-GAGCCAGUUGUCAAGAAGAUU-3′) (SEQ ID NO:3431). The siRNA was synthesized and obtained from Qiagen.

For in ovo delivery, the siRNA was formulated with lipoefectamine 2000 (Invitrogen) according to the manufacturer's instructions. The now complexed siRNA was then delivered in ovo at a dose of either 100 μmol or 200 μmol. The siRNA was injected into embryonated eggs via an intravenous (I.V.) route or directly into the amnion at embryonic day 4.5 (E4.5). For both I.V. and amnion delivery, a small opening (1 cm×1 cm) was created at the top of the blunt end of the egg so as to avoid the membrane, veins and arteries, and 100 pmol or 200 pmol in a 4 μl volume was then injected directly into a vein or into the amnionic cavity using a micro-capillary pipette. Micro-capillaries of 1 mm diameter were used for injections, and their tips were pulled to a diameter of 40 microns with bevelled tip of 22.5°. After injection, the holes in the eggs were sealed with appropriate sized parafilm squares using a heated scalpel blade.

In total, 286 embryonated eggs (E4.5) were used in this experiment;

Group 1: 48 eggs were used as controls and were not injected with the DMRT1-343-siRNA formulation;

Group 2: 51 eggs were injected I.V. with 100 pmol of siRNA;

Group 3: 53 eggs were injected I.V. with 200 pmol of siRNA;

Group 4: 81 eggs were injected into the amnion with 100 pmol of siRNA and;

Group 5: 53 eggs were injected into the amnion with 200 pmol of siRNA.

All embryos were incubated until day E10. At E10, all embryos were assessed for viability and then removed from the egg. Control Group 1 had an embryo viability of 100%; Group 2 had a viability of 76%; Group 3 had a viability of 94%; Group 4 had a viability of 40% and; Group 5 had a viability of 75%. A single limb bud from each embryo was removed and used in a sex determination PCR test to determine if the embryos were of male or female genotype. Lower limb buds from each embryo were collected into 50 μl of PCR digestion buffer (50 mM KCl; 10 mM Tris-HCl, pH8.3; 0.1 mg/ml gelatine; 0.45% Nonidet P-40; 0.45% Tween-20; 0.2 mg/ml proteinase K; stock stored at −20° C.) at room temperature and digested at 55° C. for a minimum of 1 h, then at 95° C. for 10 min to release genomic DNA.

Sexing was carried out by PCR using the method of Clinton et al. (2001). The PCR mix consisted of 1 μl of digestion mix, 10×RedTaq reaction buffer (Sigma-Aldrich), MgCl₂ to 1.5 mM (Promega), 1 unit of RedTaq DNA polymerase (Sigma-Aldrich) and Milli-Q water (Millipore) to a total volume of 20 μl. Reactions were carried out in a Master cycler S (Eppendorf) PCR machine. Products were run on a 1.5% 1×Tris-borate (TBE) agarose gels.

Once the sex PCR test was complete and analysed, the embryos were definitively labelled as either being genotypically male or female. The embryos were then opened via dissection and the gonads exposed for macroscopic analysis of gonadal development. The gonadal development of all control embryos was normal as expected. Control female embryo's showed typical asymmetric development that was characterised by a large left ovary and smaller regressing right gonad. Control male embryos all had typical bilateral testes. All female embryos from the siRNA knockdown groups (Groups 2-5) had normal gonadal development. In contrast, some male embryos from the siRNA knockdown groups showed varying degrees of female-like asymmetry at the macroscopic level of the gonads. The feminisation effect of the DMRT1-343-siRNA was characterised by an average or small-sized right testis and a larger feminised left gonad (Table 7). Feminisation was observed in a number of male embryos in Groups 2, 3 and 5 and resulted in an increase in the ratio of embryos with female-like gonads in these groups.

TABLE 7
DMRT1 embryo injection results.

| | | | | % |
|---|---|---|---|---|
| | | | | Male:Female |
| siRNA dose | No. | | % Male:Female | Macroscopic |
| and injection | embryos | No. viable at | Genotype | Gonad |
| route | injected | E10 | (PCR sex test) | phenotype |
| Group 1 | 48 | 48 (100%) | 60:40 | 60:40 |
| No injection | | | | |
| control | | | | |
| Group 2 | 51 | 39 (76%) | 59:41 | 54:46 |
| I.V.—100 pmol | | | | |
| Group 3 | 53 | 50 (94%) | 54:46 | 46:54 |
| I.V.—200 pmol | | | | |
| Group 4 | 81 | 33 (40%) | 48:52 | 48:52 |
| Amnion—100 | | | | |
| pmol | | | | |
| Group 5 | 53 | 40 (75%) | 33:67 | 28:72 |
| Amnion—200 | | | | |
| pmol | | | | |

Gonads from both male and female embryos in each treatment groups were assessed for DMRT1 gene expression using quantitative RT-PCR analysis. Both the female and male gonads were pooled separately from each group and RNA was extracted for cDNA synthesis and qPCR analysis. The pooled gonads were added to 1 ml of Trizol and homogenised well by pipetting and vortexing at room temperature until all gonad tissue had dissolved. 200 μl of chloroform was added and mixed well by inverting the sample for 15 sec. The sample was then incubated at room temperature for 3 min and then centrifuged at 12000 g for 15 min at 4° C. The aqueous phase of the sample was then transferred to a new tube and then 500 l of isopropanol was added and mixed well by inversion. The mix was then incubated at room temperature for 10 min and then centrifuged at 12000 g for 10 min at 4° C. The supernatant was removed from the tube carefully, so as not to disturb the RNA pellet, and the pellet was then washed with 1 ml of 70% ethanol. The tube was then centrifuged at 7500 g for 5 min at 4° C. and the supernatant again was carefully removed and the RNA pellet was air dried at room temperature for 10 min. The RNA pellet was then resuspended in 25 μl of RNase-free water and the final concentration of RNA was determined using a NanoDrop ND-1000 Spectrophotometer (Thermo Scientific). RNA was reverse transcribed to complimentary DNA (cDNA) using the Promega Reverse Transcription kit (Promega). The reaction mix contained 1 μg of RNA, random hexamers (1 μl), dNTPs (2 μl), AMV reverse transcriptase (Promega) (0.5 μl) and nuclease free water added to a total reaction volume of 20 μl. The mix was incubated at 42° C. for 1 hour, followed by a 10 min incubation at 95° C. for enzyme inactivation.

cDNA was then used to quantify relative DMRT1 gene expression levels in the pooled male and female gonad samples from each treatment group. qPCR primers and probes were designed using Primer Express (Applied Biosystems) software and sequences are shown in Table 8. PCR's were set up in 20 μl reaction volumes that contained 2×TaqMan qRT PCR mastermix (Applied Biosystems), 1 μl of primer/probe mix, 1 μl of cDNA sample and made up to final volume with Nuclease free water (Promega). PCR cycling was performed at 95° C. for 1 min, followed by 40 cycles of 95° C. for 15 sec; 61° C. for 30 sec and; 68° C. for 30 sec. Ct values were obtained at a standard threshold value of 0.2 for all reactions. This threshold value corresponded to the midway point of the logarithmic phase of all amplification plots. Ct values were exported to Microsoft Excel for analysing relative gene expression using the comparative Ct method.

TABLE 8
Primer and probe sequences.

| Sequence Name | Sequence 5′-3′ |
|---|---|
| DMRT1 TaqMan MGB probe | CCATCCCTTTCATCTGCC |
| | (SEQ ID NO: 3494) |
| DMRT1 Forward primer | TCAAGCCAGTCAGGAAAACAGT |
| | (SEQ ID NO: 3495) |
| DMRT1 Reverse primer | TCATGGCATGGCGGTTCT |
| | (SEQ ID NO: 3496) |
| 18S rRNA TaqMan MGB probe | TGCTGGCACCAGACTTGCCCTC |
| | (SEQ ID NO: 3497) |
| 18S rRNA Forward primer | CGGCTACCACATCCAAGGAA |
| | (SEQ ID NO: 3498) |
| 18S rRNA Reverse primer | GCTGGAATTACCGCGGCT |
| | (SEQ ID NO: 3499) |

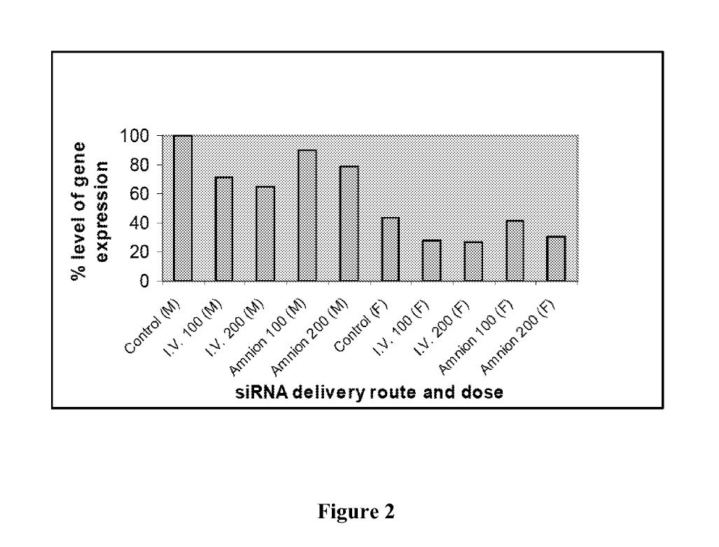

Relative levels of DMRT1 mRNA were compared with the chicken house keeping 18S rRNA species across all cDNA samples (FIG. 2). Quantitative RT-PCR analysis confirmed that DMRT1 mRNA expression was specifically reduced in all pooled groups of male embryos when compare to control Group 1. Almost 40% of DMRT1 gene expression knockdown was observed for Group 3 male embryos treated with the DMRT1-343-siRNA. It is interesting to note that Group 3 was also the group that resulted in the greatest degree of observed feminisation of male gonads at the macroscopic level.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from U.S. 61/138,235 filed 17 Dec. 2008, the entire contents of which are incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Clinton et al. (2001) British Poultry Science 42:134-138
Hori et al. (2000) Mol Biol Cell 11:3645-3660
Needleman and Wunsch (1970) J Mol Biol 48: 443-453
O'Neill et al. (2000) Dev Genes Evol 210:243-249
Raymond et al. (1999) Dev Biol 215:208-220
Smith et al. (1999) Nature 402:601-602
Smith et al. (2000) Nature 407: 319-320.
Taxman et al. (2006) BMC Biotechnol 6:7
Waterhouse et al. (1998) Proc Natl Acad Sci USA 95:13959-13964

1-20. (canceled)

21. A method of modifying the sex of an avian, the method comprising administering to an avian egg at least one isolated and/or exogenous RNA molecule comprising a double-stranded region (dsRNA) which reduces the level of a protein encoded by an R-spondin gene or Fox9 gene in the avian egg, wherein if the embryo of the egg is male the sex is altered to female following administration of the isolated and/or exogenous RNA molecule, and wherein the RNA molecule is administered to a non-cellular site of the egg and/or the egg is not electroporated.

22. A method of modifying the sex of an avian, the method comprising administering to an avian egg at least one isolated and/or exogenous RNA molecule comprising a double-stranded region (dsRNA) comprising one or more of the sequence of nucleotides provided as SEQ ID NO's 11 to 3431 or a variant of any one or more thereof, wherein the isolated and/or exogenous RNA molecule does not comprise a sequence selected from:
CCAGUUGUCAAGAAGAGCA (SEQ ID NO:254)
GGAUGCUCAUUCAGGACAU (SEQ ID NO:369)
CCCUGUAUCCUUACUAUAA (SEQ ID NO:474)
GCCACUGAGUCUUCCUCAA (SEQ ID NO:530)
CCAGCAACAUACAUGUCAA (SEQ ID NO:605)
CCUGCGUCACACAGAUACU (SEQ ID NO:747)
GGAGUAGUUGUACAGGUUG (SEQ ID NO:3432)
GACUGGCUUGACAUGUAUG (SEQ ID NO:3433)
AUGGCGGUUCUCCAUCCCU (SEQ ID NO:3434) or a variant of any one thereof, and wherein the RNA molecule is administered to a non-cellular site of the egg and/or the egg is not electroporated.

23. A method of modifying the sex of an avian, the method comprising administering to an avian egg at least one isolated and/or exogenous RNA molecule comprising a double-stranded region (dsRNA) which reduces the level of a protein encoded by an β-catenin in the avian egg, wherein the RNA molecule is administered to a non-cellular site of the egg and/or the egg is not electroporated.

24. The method of claim 21, wherein the non-cellular site is the air sac, yolk sac, amnionic cavity or chorion allantoic fluid.

25. The method of claim 22, wherein the non-cellular site is the air sac, yolk sac, amnionic cavity or chorion allantoic fluid.

26. The method of claim 23, wherein the non-cellular site is the air sac, yolk sac, amnionic cavity or chorion allantoic fluid.

27. The method of claim 21, wherein the dsRNA is a siRNA or a shRNA.

28. The method of claim 22, wherein the dsRNA is a siRNA or a shRNA.

29. The method of claim 23, wherein the dsRNA is a siRNA or a shRNA.

30. The method of claim 21, wherein the RNA molecule is administered by injection.

31. The method of claim 22, wherein the RNA molecule is administered by injection.

32. The method of claim 23, wherein the RNA molecule is administered by injection.

33. The method of claim 21, wherein the avian is selected from the group consisting of chickens, ducks, turkeys, geese, bantams and quails.

34. The method of claim 22, wherein the avian is selected from the group consisting of chickens, ducks, turkeys, geese, bantams and quails.

35. The method of claim 23, wherein the avian is selected from the group consisting of chickens, ducks, turkeys, geese, bantams and quails.

36. An avian produced using a method according of claim 21.

37. An avian produced using a method according of claim 22.

38. An avian produced using a method according of claim 23.
